(19) United States
(12) Patent Application Publication
(10) Pub. No.: US 2020/0017588 A1
(43) Pub. Date: Jan. 16, 2020

(54) MODULAR TETRAVALENT BISPECIFIC ANTIBODY PLATFORM

(71) Applicant: Dana-Farber Cancer Institute, Inc., Boston, MA (US)

(72) Inventor: Wayne A. Marasco, Wellesley, MA (US)

(21) Appl. No.: 16/340,878

(22) PCT Filed: Oct. 16, 2017
(86) PCT No.: PCT/US2017/056814

§ 371 Date: Apr. 10, 2019

Related U.S. Application Data
(60) Provisional application No. 62/408,271, filed on Oct. 14, 2016.

(51) Int. Cl. C07K 16/28; C07K 16/46

(52) U.S. Cl. CPC C07K 16/2827; C07K 16/2878; C07K 16/468; C07K 2317/31; C07K 2317/524; C07K 2319/00; C07K 2317/622; C07K 2317/64; C07K 2317/732; C07K 2317/734; C07K 2317/53

(57) ABSTRACT

The present invention relates to a tetrameric bispecific antibody molecule, as well as a method for producing the same, its use and a nucleic acid molecule encoding the tetrameric bispecific antibody molecule.

### RELATED APPLICATIONS

This application claims benefit of, and priority to, U.S. Ser. No. 62/408,271 filed on Oct. 14, 2016 the contents of which is hereby incorporated by reference its entirety.

### GOVERNMENT INTEREST

This invention was made with government support under [ ] awarded by the [ ]. The government has certain rights in the invention.

### FIELD OF THE INVENTION

The present invention relates generally to tetrameric bispecific antibody molecules, methods and systems of producing same.

### SEQUENCE LISTING

The instant application contains a Sequence Listing which has been filed electronically in ASCII format and is hereby incorporated by reference in its entirety. Said ASCII copy, created on May 30, 2019, is named 5031461-038-US2_SL.txt and is 1,002,845 bytes in size.

### BACKGROUND OF THE INVENTION

Bispecific antibodies (BsAb) are antibodies or antibody-like molecules having two different binding specificities. BsAbs have broad applications in biomedicine, especially in immunotherapy for tumors. Presently, a focus of immunotherapy research is on how to utilize cell-mediated cytotoxicity of BsAb to kill tumor cells. A BsAb can be designed to target a tumor cell and an effector cell simultaneously, while triggering the effector cell's destruction of the tumor cell.

BsAb can be prepared by methods such as chemical engineering, cell engineering and genetic engineering. An advantage of genetic engineering is that the antibodies can be easily modified, which renders design and production of many different forms of bispecific antibody fragments, including diabodies, tandem ScFv, and single-chain diabodies, as well as derivatives thereof. Since those BsAbs do not have an IgG Fc domain, their small size enhances their penetration into tumors, but they have significantly shorter half-life in vivo and also lack the ADCC effect that is associated with the constant region of the antibody.

To improve the stability and therapeutic potential, recombinant genetic modifications were made in the heavy chains to facilitate their heterodimerization and to produce greater yields of Fc-containing IgG-like bispecific antibodies. Several rational design strategies have been used to engineer antibody CH3 chains for heterodimerization, namely disulfide bonds, salt bridges, knobs-into-holes. The bases for creating knob and hole in the juxtaposed positions is that the knob and hole interaction will favor heterodimer formation, whereas the knob-knob and the hole-hole interaction will prevent homodimers formation due to the deletion of favorable interactions. While this knob-into-holes approach solves the heavy chain homodimerization problem, it did not address the issues regarding mispairing between the light chain and heavy chains from two different antibodies. Although it is possible to identify identical light chains for two different antibodies, the possibility of BsAb construction using two antibody sequences that can share the common light chain is very limited.

There is a need to provide better BsAbs that are easier to prepare, have better clinical stability and efficacy and/or reduced systematic toxicity.

### SUMMARY OF THE INVENTION

The present invention provides tBsAbs that are easier to prepare, have better clinical stability and efficacy, and/or reduced systematic toxicity.

One aspect of the present invention relates to a tetravalent antibody molecule. The tetravalent antibody may be a dimer of a bispecific scFv fragment including a first binding site for a first antigen, a second binding site for a second antigen. The two binding sites may be joined together via a linker domain. In embodiments, the scFv fragment is a tandem scFv, the linker domain includes an immunoglobulin hinge region (e.g., an IgG1, an IgG2, an IgG3, and an IgG4 hinge region) amino acid sequence. In embodiments, the immunoglobulin hinge region amino acid sequence may flanked by a flexible linker amino acid sequence, e.g., having the amino acid sequence (GGGS)_{X1-6} (SEQ ID NO: 1906), (GGGGS)_{X1-6} (SEQ ID NO: 1907), and GSAGSAAGSGEF (SEQ ID NO: 1908). In embodiments, the linker domain includes at least a portion of an immunoglobulin Fc domain, e.g., an IgG1, an IgG2, an IgG3, and an IgG4 Fc domain. The at least a portion of the immunoglobulin Fc domain may be a CH2 domain. The Fc domain may be linked to the C-terminus of an immunoglobulin hinge region (e.g., an IgG1, an IgG2, an IgG3, and an IgG4 hinge region) amino acid sequence. The linker domain may include a flexible linker amino acid sequence (e.g., (GGGS)_{X1-6} (SEQ ID NO: 1906), (GGGGS)_{X1-6} (SEQ ID NO: 1907), and GSAGSAAGSGEF (SEQ ID NO: 1908)) at one terminus or at both termini.

Another aspect, the present invention relates to nucleic acid construct. The construct may include nucleic acid molecules encoding: a light chain variable region and a heavy chain variable region of an antibody that can specifically bind to a first antigen; a light chain variable region and a heavy chain variable region of an antibody that can specifically bind to a second antigen; and a linker domain. In embodiments, the linker domain is an immunoglobulin hinge region (e.g., an IgG1, an IgG2, an IgG3, and an IgG4 hinge region) amino acid sequence. In embodiments, the linker domain is at least a portion of an immunoglobulin Fc domain, e.g., an IgG1, an IgG2, an IgG3, and an IgG4 Fc domain. The at least a portion of the immunoglobulin Fc domain may be a CH2 domain. The Fc domain may be linked to the C-terminus of an immunoglobulin hinge region (e.g., an IgG1, an IgG2, an IgG3, and an IgG4 hinge region) amino acid sequence. The linker domain may include a flexible linker amino acid sequence (e.g., (GGGS)X1-6 (SEQ ID NO: 1906), (GGGGS)X1-6 (SEQ ID NO: 1907), and GSAGSAAGSGEF (SEQ ID NO: 1908)) at one terminus or at both termini.

Yet another aspect of the present invention is a vector including the nucleic acid construct of the above aspect.

Another aspect of the present invention is a host cell (e.g., a T-cell, a B-cell, a follicular T-cell, and an NK-cell) which includes the vector of the above aspect.

An aspect of the present invention is a chimeric antigen receptor (CAR). The CAR may include an intracellular signaling domain, a transmembrane domain and an extracellular domain including the tetravalent antibody molecule of any of the above aspects or embodiments. In embodiments, the transmembrane domain further includes a stalk region positioned between the extracellular domain and the transmembrane domain and/or the transmembrane domain comprises CD28. In embodiments, the CAR further includes one or more additional costimulatory molecules (e.g., CD28, 4-1BB, ICOS, and OX40) positioned between the transmembrane domain and the intracellular signaling domain, e.g., a CD3 zeta chain.

Yet another aspect of the present invention is a genetically engineered cell. The genetically engineered cell may express and bear on its cell surface membrane the chimeric antigen receptor of an above aspect or embodiment. In embodiments, the cell is a T-cell (e.g., CD4+ and/or CD8+) or an NK cell. The cell may comprise a mixed population of CD4+ and CD8 cells+.

An aspect of the present invention is method for treating a disease or disorder. The method may include administering the tetravalent antibody molecule of an above aspect or embodiment. In embodiments, the disease or disorder is a CNS-related disease or disorder, e.g., a CNS cancer or a neurodegenerative disease. The CNS cancer may be a Glioblastoma (GBM). The neurodegenerative disease may be Amyotrophic Lateral Sclerosis, Parkinson's Disease, Alzheimer's Disease, or Huntington's Disease. In embodiments, the tetravalent antibody molecule recognizes and/or is bound by a CNS transport receptor, e.g., a transferrin receptor (TfR), VCAM-1, CD98hc, and an insulin receptor. In this aspect and any above aspect or embodiment, the tetravalent antibody molecule augments transport across the blood brain barrier.

Any of the above aspects or embodiments may be combined with any other aspect or embodiment.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is an illustration showing the design and formation of a tetrameric bispecific antibody (tBsAb).

FIG. 2 is a schematic representation of the pcDNA3.1\1 scFv-hinge—scFv expression vector.

FIG. 3A is an SDS gel showing a synthetic tetramer linker that was digested using NotI and BsiWI and then inserted into tetramer expressing vector. FIG. 3B is an SDS gel showing the purification of a tBsAb according to the invention.

FIG. 4A is an illustration showing the method for detection of antibody binding affinity of a tBsAb. FIG. 4B to FIG. 4C are graphs showing data when plates were coated with CCR4-Fc (B) or with PD-L1-Fc (C) and then incubated with tetravalent bispecific (anti-CCR4 and anti-PD-L1) and control antibodies. The result showed this tetravalent antibody could bind to both CCR4-Fc and PD-L1-Fc in a dose dependent manner.

FIG. 5 is a graph showing that anti-CAIX-PD-L1 bispecific mAb binds to CAIX-Fc fusion protein.

FIG. 6 is a graph showing that anti-CAIX-PD-L1 bispecific mAb binds to PD-L1-Fc fusion protein.

FIG. 7A is an illustration of how linker lengths can be changed to optimize bispecific mAb binding. FIG. 7A discloses SEQ ID NOS 1910-1912, respectively, in order of appearance. FIG. 7B is a schematic of a tBsAb sequence. FIG. 7B discloses SEQ ID NOS 1913-1915, respectively, in order of appearance.

FIG. 8A. αGITR-αPD-L1 tBsAb engagement. The tBsAb binds to the GITR protein on T cells and PD-L1 protein on tumor cells. B. Schematic representation of the tBsAb format which is achieved through interchain disulfide bond formation between cysteine residues of the hinge region.

FIG. 9A. Basic structure of the two scFvs linked to form the tBsAb. Figure discloses “His6” as SEQ ID NO: 1084. FIG. 9B. Bispecific dimeric taFv directed against GITR and PD-L1. Each V_{H} and V_{L} pair is connected by a linker of 15 residues to form scFv. Two scFv are connected by a linker-hinge-linker (55 residues). The hinge region has two cysteine residues allowing the pairing of two taFv through disulfide bridges under oxidative conditions.

FIG. 10A. Basic structure of the tandem scFv. Figure discloses “His6” as SEQ ID NO: 1084. FIG. 10B. Trifunctional tBsAb directed against GITR and PD-L1 with an additional CH2 domain. Each V_{H} and V_{L} pair is connected by linker of 15 residues. Two scFv are connected by a linker-hinge-CH2-linker domain. The hinge region has two cysteine residues allowing the pairing of two tandem scFv through disulfide bridges under oxidative conditions. The N-terminal end of the CH2 domain can bind Fc-γ or C1q. The resulting format is a trifunctional tBsAb.

FIG. 11 Mechanism of action of αGITR-αPD-L1 tBsAb. FIG. 11A. Tumor cells overexpress the PD-L1 protein. The PD-1/PD-L1 interaction inhibits an effective T cell activation and promotes immune suppression and adaptive immune resistance. FIG. 11B. The αGITR-αPD-L1 tBsAb may enhance immune response. The αPD-L1 arm blocks PD-1/PD-L1 pathway and may therefore inhibit T cell exhaustion and abrogate T reg suppression. The αGITR arm acts as an agonist on the co-stimulator GITR receptor, resulting in upregulation of GITR expression enhancing T cell activation and proliferation.

FIG. 12 Schematic representation of the αGITR-αPD-L1 cloning process. The donor vector and the pcDNA 3.4 expression vector were digested with SfiI and NotI restriction enzymes. The V_{H}GITR-V_{L}GITR gene was isolated and subsequently ligated to each other. The final plasmid resulted in the αGITR-αPD-L1 clone.

FIG. 13 Schematic representation of the control plasmid (1) cloning process. The pcDNA3.1 vector and the expression vector pcDNA 3.4 were digested with SfiI and NotI restriction enzymes. The V_{H}F10-V_{L}F10 gene was isolated and subsequently ligated to the pcDNA 3.4 expression vector. The final plasmid resulted in the αGITR-αPD-L1 clone.

FIG. 14 Schematic representation of the control plasmids (2) and (3) cloning process. The isolated F10 V_{H} and V_{L} DNA and the recipient vector pcDNA 3.4 were digested with BsiWI and BamHI restriction enzymes and subsequently ligated to each other. The final plasmid resulted in the final αGITR1-αPD-L1 and αGITR10-αPD-L1 clone.

FIG. 15 Schematic representation of the cloning strategy for the αGITR-αPD-L1 with CH2 construct. The HindIII restriction site was introduced into the pcDNA 3.4 expression vector by site directed mutagenesis. The isolated CH2 fragment and the expression vector were subsequently digested and ligated to each other to finalize the construct αGITR-αPD-L1 with CH2.

FIG. 16 Restriction enzyme analysis (REA) of recipient pcDNA 3.4 vector, six V_{H}GITR-V_{L}GITR inserts and one V_{H}F10-V_{L}F10 insert. Shown is an ethidium bromide stained 1% agarose gel of DNA electrophoresed in TAE buffer. All plasmids were digested with SfiI and NotI restriction enzymes. Lane 1: shown is a 7.5 kb digested recipient pcDNA 3.4 vector. The lower band between 500 and 1000 bp is a previously-used scFv insert (from the Marasco Laboratory). Lanes 2-6: Lower bands represent the 800 bp V_{H}GITR-linker-V_{L}GITR inserts. The larger bands clustered at 8 kb are the double digested descendent vectors. Lane 7: The 800 bp V_{H}F10-linker-V_{L}F10 inserts is visualized in the lower band clustered between 500 and 1000 bp. The lane “bp” represents the 1 kb DNA ladder (NEB).

FIG. 17 REA of V_{H}F10-linker-V_{L}F10 cDNA and recipient pcDNA 3.4 expression (containing V_{H}GITR1-V_{L}GITR1 or V_{H}GITR10-V_{L}GITR10, respectively). Shown is an ethidium bromide stained 1% agarose gel of DNA electrophoresed in TAE buffer. The recipient expression vectors and the insert were digested with BsiWI and BamHI restriction enzymes. Lane 1: shown is a single band clustered at 800 bp, representing the V_{H}F10-linker-V_{L}F10 (scFv) fragment isolated by PCR. Lanes 2 and 3: The upper two bands visualize the pcDNA 3.4 expression vectors containing V_{H}GITR1-V_{L}GITR1 (lane 2) and V_{H}GITR10-V_{L}GITR10 (lane 3). Both comprise 7500 bp and can be detected at the correct level of the ladder. The lower bands in lanes 2 and 3 clustered between 500 and 1000 bp represent the digested V_{H}PD-L1-V_{L}PD-L1 fragment separated from its vector. The lane “bp” corresponds to the 1 kb DNA ladder (NEB).

FIG. 18 Analysis of purified tBsAbs by SDS-PAGE. Shown is a Coomassie blue stained SDS gel of protein electrophoresed in MES buffer. 3-5 μg of protein sample was loaded and separated on the gel under (A) reducing and (B) non-reducing conditions. Lanes 1-8: Under non-reducing conditions, the SDS PAGE revealed two major bands of each protein. The higher bands have an apparent molecular weight between 80 kDa and 115 kDa and the lower molecular weight bands between 70 and 80 kDa. In the non-reduced SDS-gel analysis, some weak but high molecular weight bands (>180 kDa) can be observed. The SDS-gel analysis under reducing conditions (10% DTT; 70° C. for 10 minutes) displays only one band with an apparent molecular weight between 70 and 80 kDa. Lane 9: Under non-reducing conditions, a single band is shown with an apparent molecular weight slightly over 140 kDa. The visualization of two bands under reducing conditions emphasizes the correct expression of αGITR IgG that display separated heavy and light chains (50 kDa and 25 kDa). The lanes kDa represent the benchmark pre-stained protein ladder (Invitrogen) under the corresponding conditions (4-12% gel concentration run in MES buffer).

FIG. 19 ELISA absorbance values of αGITR-αPD-L1 tBsAbs, F10-αPD-L1 tBsAb and αPD-L1 mAb tested against passively immobilized PD-L1 antigen. A concentration range of each αGITR-αPD-L1 (0.0001 mg/mL-1 mg/mL; horizontal axis) was subjected to ELISA on PD-L1 antigen. The results show the mean and standard deviation of the absorbance at 450 nm (vertical axis). Each sample was run in triplicates at every concentration. The raw signal intensity was corrected for the background signal by subtracting the mean signal of wells incubated in the absence of the primary antibody from the wells where primary antibody was added.

FIG. 20 Cell-based ELISA testing αGITR1-αPD-L1 and αGITR10-αPD-L1 antibodies binding against GITR+ expressing CF2 cells fixed with acetone-methanol. The F10-αPD-L1 antibody represents the negative control. All antibodies were tested using a range of serial 1:3 dilutions ranging from 3.3 mg/mL to 0.0046 mg/mL. All antibodies were tested against 1000 GITR+CF2 cells per well. Each bar represents an average obtained from triplicate samples (deviations indicated by bars). The raw signal intensity was corrected for the background signal by subtracting the mean signal of wells incubated in the absence of the primary antibody from the wells where primary antibody was added.

FIG. 21 Cell based ELISA testing αGITR1-αPD-L1 and αGITR10-αPD-L1 antibodies binding against GITR+ expressing CF2 cells fixed with 8% paraformaldehyde. The F10-αPD-L1 antibody represents the negative control. All antibodies were tested using a range of serial 1:3 dilutions ranging from 3.3 mg/mL to 0.0046 mg/mL. All antibodies were tested against 1000 GITR+CF2 cells per well. Each bar represents an average obtained from triplicate samples (deviations indicated by bars). The raw signal intensity was corrected for the background signal by subtracting the mean signal of wells incubated in the absence of the primary antibody from the wells where primary antibody was added.

FIG. 22 Cell-based ELISA testing αGITR10-αPD-L1 and commercial αGITR10 mAb antibodies binding against GITR+ expressing CF2 cells fixed with 8% paraformaldehyde. The F10-αPD-L1 antibody represents the negative control. All antibodies were tested using a range of serial 1:2 dilutions ranging from 5 mg/mL to 0.078 mg/mL. All antibodies were tested against 10,000 GITR+CF2 cells per well. Each bar represents an average obtained from triplicate samples (deviations indicated by bars). The raw signal intensity was corrected for the background signal by subtracting the mean signal of wells incubated in the absence of the primary antibody from the wells where primary antibody was added.

FIG. 23A. Flow cytometric analysis of fluorescent-activated αGITR10-αPD-L1 tBsAb (anti-His Alexa 488 (APC) conjugated) tested with GITR+CF2 cells. FIG. 23B. Flow cytometric analysis of fluorescent-activated αGITR10 IgG Ab (anti human IgG Fc (FITC conjugated) tested with GITR+CF2 cells. Horizontal lines indicate the intensity signal of fluorescence and the vertical axis indicates the cell counts. Each individual picture represents different concentration of αGITR10-αPD-L1 with constant cell number.

FIG. 24A. Flow cytometric analysis of fluorescent-activated αGITR1-αPD-L1 tBsAb (anti-His Alexa 488 (APC) conjugated) tested with GITR+CF2 cells. FIG. 24B. Flow cytometric analysis of fluorescent-activated αGITR10 IgG Ab (anti human IgG Fc (FITC conjugated) tested with GITR+CF2 cells. Horizontal lines indicate the intensity signal of fluorescence and the vertical axis indicates the cell counts. Each individual picture represents different concentration of αGITR10-αPD-L1 (FIG. 24A) and αGITR10 IgG (FIG. 24B) with constant cell number.

FIG. 25 Restriction enzyme analysis (REA) of 16 clones. Shown is an ethidium bromide stained 1% agarose gel of DNA electrophoresed in TAE buffer. All plasmids were digested with HindIII and BamHI restriction enzymes. Two bands are shown in lane No.10: The band clustered between 6 kb and 8 kb represents the digested recipient pcDNA 3.4 vector (7.5 kb). The lower band between 500 and 1000 bp indicates a close approximation to the expected theoretical size of 800 bp of the fragment isolated with HindIII and BamHI restriction enzymes. The lane “bp” represents the 1 kb DNA ladder.

FIG. 26 REA of clone 10 (GITR10-PDL1 with HindIII) and GITR10-PDL1 (without HindIII restriction site). Shown is an ethidium bromide stained 1% agarose gel of DNA electrophoresed in TAE buffer. Both plasmids were digested with only HindIII (lane 1), only NotI (lane 2) and with HindIII and NotI simultaneously (lane 3). The digestions of clone No.10 with a single enzyme (lanes 1 & 2) resulted one band clustered around 8000 bp. The digestion of clone No. 10 with both enzymes (lane 3) resulted in the generation of two fragments, of which the smaller-sized band is clustered below 500 bp. The digestion of αGITR10-αPD-L1 with only HindIII restriction site (lane 1), revealed a supercoiled plasmid DNA.

FIG. 27 Restriction enzyme digestion analysis of vector GITR10-PDL1 (containing HindIII restriction site) and CH2 fragment. Shown is an ethidium bromide stained 1% agarose gel of DNA electrophoresed in TAE buffer. Lane 1: single digestion of the αGITR-αPD-L1 with HindIII. Lane 2: The CH2 fragment digested with HindIII resulted in a band clustered below the 500 bp mark of the ladder. The lane “bp” corresponds to the 1 kb DNA ladder.

FIG. 28 Analysis of purified αGITR10-αPD-L1 with CH2 BsAb by SDS-PAGE. Shown is a Coomassie blue stained SDS gel of protein electrophoresed in MES buffer. 3-5 μg of protein sample was loaded and separated on the gel under (R) reducing and (NR) non-reducing conditions. Under non-reducing conditions, the SDS PAGE revealed two major bands of each protein. The higher band has an apparent molecular of around 140 kDa and the lower hand has a molecular weight of 80 kDa, correlating with the theoretical size of dimeric (150 kDa) and monomeric (75 kDa) BsAbs. The SDS-gel analysis under reducing conditions (10% DTT; 70° C. for 10 minutes) displays only one band with an apparent molecular weight between around 80 kDa and reinforces the correct expression of tBsAb that can be reduced by its disulfide bridges in the hinge region. The lane “kDa” represents the benchmark pre-stained protein ladder (Invitrogen) under the corresponding conditions (4-12% gel concentration run in MES buffer).

FIG. 29 Cell-based ELISA testing αGITR10-αPD-L1 with CH2 and αGITR10 IgG antibodies binding against GITR+ expressing CF2 cells fixed with 8% paraformaldehyde. The F10-αPD-L1 antibody represents the negative control. All antibodies were tested using a range of serial 1:2 dilutions ranging from 5 mg/mL to 0.16 mg/mL. All antibodies were tested against 10,000 GITR+CF2 cells per well. Each bar represents an average obtained from triplicate samples (deviations indicated by bars). The raw signal intensity was corrected for the background signal by subtracting the mean signal of wells incubated in the absence of the primary antibody from the wells where primary antibody was added.

FIG. 30 ADCC activity of αGITR10-αPD-L1 with CH2 antibody. The ADCC activity of αGITR10-αPD-L1 with CH2 and was measured at varying concentrations. All antibodies were serially diluted (1:2), starting the highest concentration at 20 mg/mL until 0.02 mg/mL and tested against 20,000 GITR+CF2 cells per well. The ratio of effector cells (GITR+CF2) to target cells (Wils-2) was 5:1. The αGITR IgG represents the positive control and F10-αPD-L1 the negative control. The vertical axis represents the raw value of luciferase activity in the effector cell quantified with luminescence readout. Each sample was run in triplicate at every concentration; the mean standard deviation is indicated in brackets. The background of GITR+CF2 cells in RPMI medium was subtracted from the obtained values.

FIG. 31 αGITR10-αPD-L1 with CH2 antibody mediated CDC activity via mouse complement. Percentage of GITR+CF2 cell lysis obtained with serial dilutions of αGITR10-αPD-L1 tBsAb and the controls αGITR mAb (positive), αGITR10-αPD-L1 (negative) determined by the CDC assay. All antibodies were serially diluted (1:10), starting the highest concentration at 20 mg/mL until 0.2 mg/mL and tested against 10,000 GITR+CF2 cells per well. The vertical axis represents the percentage of lysis. It is calculated as ratio of obtained sample signal to the signal intensity from fully lysed GITR+CF2 cells. Each sample was run in triplicate at every concentration; the mean standard deviation is indicated in brackets. The background of GITR+CF2 cells in RPMI medium was subtracted from the obtained values. Each bar represents the simple average obtained from triplicate samples (standard deviations indicated by brackets).

FIG. 32 Cell-based ELISA testing αGITR1-αPD-L1 and αGITR10-αPD-L1 antibodies binding against CF2 cells (without GITR expression) fixed with 8% paraformaldehyde. The F10-αPD-L1 antibody represents the negative control. All antibodies were tested using a range of serial 1:3 dilutions ranging from 3.3 mg/mL to 0.0046 mg/mL. All antibodies were tested against 1000 GITR-CF2 cells per well. Each bar represents an average obtained from triplicate samples (deviations indicated by bars). The raw signal intensity was corrected for the background signal by subtracting the mean signal of wells incubated in the absence of the primary antibody from the wells where primary antibody was added.

FIG. 33 Cell-based ELISA testing αGITR10-αPD-L1 and αGITR10 IgG antibodies binding against CF2 cells (without GITR expression) fixed with 8% paraformaldehyde. The F10-αPD-L1 antibody represents the negative control. All antibodies were tested using a range of serial 1:2 dilutions ranging from 5 mg/mL to 0.078 mg/mL. All antibodies were tested against 10,000 GITR-CF2 cells per well. Each bar represents an average obtained from triplicate samples (deviations indicated by bars). The raw signal intensity was corrected for the background signal by subtracting the mean signal of wells incubated in the absence of the primary antibody from the wells where primary antibody was added.

FIG. 34 Cell-based ELISA testing αGITR10-αPD-L1 with CH2 and αGITR10 IG antibodies binding against GITR−CF2 cells fixed with 8% paraformaldehyde. The F10-αPD-L1 antibody represents the negative control. All antibodies were tested using a range of serial 1:2 dilutions ranging from 5 mg/mL to 0.16 mg/mL. All antibodies were tested against 10,000 GITR+CF2 cells per well. Each bar represents an average obtained from triplicate samples (deviations indicated by bars). The raw signal intensity was corrected for the background signal by subtracting the mean signal of wells incubated in the absence of the primary antibody from the wells where primary antibody was added.

FIG. 35 Control set-up for Flow cytometric analysis of fluorescent-activated αGITR1-αPD-L1 antibody. Lanes 1 to 6 show the controls set up for GITR+ CF2 cells. Lanes 7 to 9 refer to the controls set up for GITR− CF2 cells.

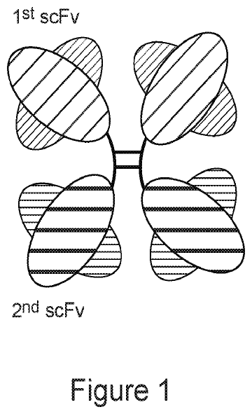

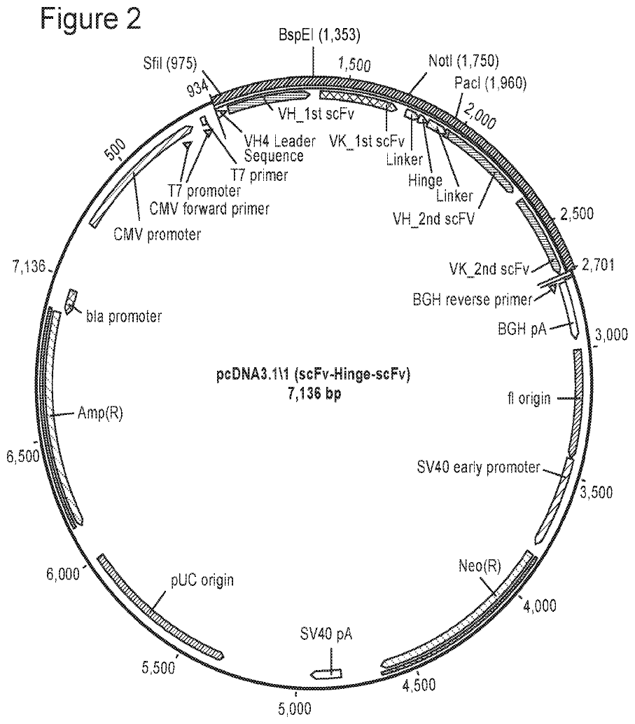

### DETAILED DESCRIPTION OF THE INVENTION

The invention relates to bispecific antibody contains two binding sites for each receptor (i.e. a tetravalent bispecific antibody or “tBsAb”), systems and methods of producing same.

The clinical development of bispecific antibodies (BsAb) as therapeutics has been hampered by the difficulty in preparing the materials in sufficient quantity and quality by traditional methods. In recent years, a variety of recombinant methods have been developed for efficient production of BsAb, both as antibody fragments and as full-length IgG-like molecules. These recombinant antibody molecules possess dual antigen-binding capability with, in most cases, monovalency to each of their target antigens. The present invention provides an efficient approach for the production of a novel tetravalent BsAb (tBsAb), with two antigen-binding sites to each of its target antigens, genetically engineering a scFV bispecific antibody and fusing the two together.

Compared to the bispecific/divalent antibody, the tBsAb binds more efficiently to both of its target antigens and is more efficacious in blocking ligand binding to the receptors. Additionally, expression of the tBsAb in mammalian cells yielded higher level of production and better antibody activity. Importantly, the tBsAbs' exhibit higher stability and longer half-life compared to monovalent bispecific antibodies. One drawback of monovalent bispecific antibodies is their small size and therefor short serum half-life requiring administration in continuous low doses for several weeks. In contrast, the longer half-life of the tBsAbs of the invention solves this problem and therefore more suitable for clinical applications. This design and expression for tBsAb should be applicable to any pair of antigen specificities.

Preferably, the tBsAb is specific for BMCA, CAIX, CCR4, PD-L1, PD-L2, PD1, Glucocorticoid-Induced Tumor Necrosis Factor Receptors (GITR), Severe acute respiratory syndrome (SARS), influenza, flavivirus or Middle East Respiratory Syndrome (MERS).

Exemplary antibodies useful in constructing the tBsAb according to the invention includes antibodies disclosed in for example: WO/2005/060520, WO/2006/089141, WO/2007/065027, WO/2009/086514, WO/2009/079259, WO/2011/153380, WO/2014/055897, WO 2015/143194, WO 2015/164865, WO 2013/166500, WO 2014/144061, WO 2016/057488, WO 2016/054638, WO/2016/164835, PCT/US2016/026232, PCT/US2017/050093, PCT/US2017/050327 and PCT/US2017/043504 the contents of which are hereby incorporated by reference in their entireties.

PDL1 (68)

Exemplary anti-PDL1 antibodies include antibodies having a _{VH} nucleotide sequence having SEQ ID NO: 1485 and a _{VL} nucleotide sequence having SEQ ID NO: 1487; a VH nucleotide sequence having SEQ ID NO: 1485 and a VL nucleotide sequence having SEQ ID NO: 1487; a VH nucleotide sequence having SEQ ID NO: 1489 and a VL nucleotide sequence having SEQ ID NO: 1491; a VH nucleotide sequence having SEQ ID NO: 1493 and a VL nucleotide sequence having SEQ ID NO: 1495; a VH nucleotide sequence having SEQ ID NO: 1497 and a VL nucleotide sequence having SEQ ID NO: 1499; a VH nucleotide sequence having SEQ ID NO: 1501 and a VL nucleotide sequence having SEQ ID NO: 1503; a VH nucleotide sequence having SEQ ID NO: 1505 and a VL nucleotide sequence having SEQ ID NO: 1507; a VH nucleotide sequence having SEQ ID NO: 1509 and a VL nucleotide sequence having SEQ ID NO: 1511; a VH nucleotide sequence having SEQ ID NO: 1513 and a VL nucleotide sequence having SEQ ID NO: 1515; a VH nucleotide sequence having SEQ ID NO: 1517 and a VL nucleotide sequence having SEQ ID NO: 1519; a VH nucleotide sequence having SEQ ID NO: 1521 and a VL nucleotide sequence having SEQ ID NO: 1523; a VH nucleotide sequence having SEQ ID NO: 1525 and a VL nucleotide sequence having SEQ ID NO: 1527; a VH nucleotide sequence having SEQ ID NO: 1529 and a VL nucleotide sequence having SEQ ID NO: 1531; a VH nucleotide sequence having SEQ ID NO: 1533 and a VL nucleotide sequence having SEQ ID NO: 1535; a VH nucleotide sequence having SEQ ID NO: 1537 and a VL nucleotide sequence having SEQ ID NO: 1539.

Exemplary anti-PDL1 antibodies include antibodies having a _{VH} amino acid sequence having SEQ ID NO: 970 and a _{VL} amino acid sequence having SEQ ID NO: 971; a VH amino acid having SEQ ID NO: 1486 and a VL polypeptide sequence having SEQ ID NO: 1488 a VH amino acid having SEQ ID NO: 1490 and a VL polypeptide sequence having SEQ ID NO: 1492 a VH amino acid having SEQ ID NO: 1494 and a VL polypeptide sequence having SEQ ID NO: 1496 a VH amino acid having SEQ ID NO: 1498 and a VL polypeptide sequence having SEQ ID NO: 1500 a VH amino acid having SEQ ID NO: 1502 and a VL polypeptide sequence having SEQ ID NO: 1504 a VH amino acid having SEQ ID NO: 1506 and a VL polypeptide sequence having SEQ ID NO: 1508 a VH amino acid having SEQ ID NO: 1510 and a VL polypeptide sequence having SEQ ID NO: 1512 a VH amino acid having SEQ ID NO: 1514 and a VL polypeptide sequence having SEQ ID NO: 1516 a VH amino acid having SEQ ID NO: 1518 and a VL polypeptide sequence having SEQ ID NO: 1520 a VH amino acid having SEQ ID NO: 1522 and a VL polypeptide sequence having SEQ ID NO: 1524 a VH amino acid having SEQ ID NO: 1526 and a VL polypeptide sequence having SEQ ID NO: 1528 a VH amino acid having SEQ ID NO: 1530 and a VL polypeptide sequence having SEQ ID NO: 1532 a VH amino acid having SEQ ID NO: 1534 and a VL polypeptide sequence having SEQ ID NO: 1536 a VH amino acid having SEQ ID NO: 1538 and a VL polypeptide sequence having SEQ ID NO: 1540.

In other embodiments the anti-PDL1 antibodies have a heavy chain with three CDRs including the amino acid sequences SEQ ID NO: 1541, 1554, 1569 respectively and a light chain with three CDRs including the amino acid sequences 1584, 1599, 1610 respectively; or a heavy chain with three CDRs comprising the amino acid sequences 1543, 1556, 1571 and a light chain with three CDRs comprising the amino acid sequences 1586, 1600, 1612; or a heavy chain with three CDRs comprising the amino acid sequences 1544, 1557, 1572 and a light chain with three CDRs comprising the amino acid sequences 1587, 1601, 1613; or a heavy chain with three CDRs comprising the amino acid sequences 1545, 1558, 1573 and a light chain with three CDRs comprising the amino acid sequences 1588, 1602, 1614; or a heavy chain with three CDRs comprising the amino acid sequences 1546, 1559, 1574 and a light chain with three CDRs comprising the amino acid sequences 1589, 1603, 1615; or a heavy chain with three CDRs comprising the amino acid sequences 1547, 1560, 1575 and a light chain with three CDRs comprising the amino acid sequences 1590, 1604, 1616; or a heavy chain with three CDRs comprising the amino acid sequences 1548, 1561, 1576 and a light chain with three CDRs comprising the amino acid sequences 1591, 1605, 1617; or a heavy chain with three CDRs comprising the amino acid sequences 1541, 1562, 1577 and a light chain with three CDRs comprising the amino acid sequences 1592, 1599, 1618; or a heavy chain with three CDRs comprising the amino acid sequences 1549, 1563, 1578 and a light chain with three CDRs comprising the amino acid sequences 1593, 1606, 1619; or a heavy chain with three CDRs comprising the amino acid sequences 1550, 1564, 1579 and a light chain with three CDRs comprising the amino acid sequences 1594, 1607, 1620; or a heavy chain with three CDRs comprising the amino acid sequences 1551, 1565, 1580 and a light chain with three CDRs comprising the amino acid sequences 1595, 1599, 1621; or a heavy chain with three CDRs comprising the amino acid sequences 1542, 1566, 1581 and a light chain with three CDRs comprising the amino acid sequences 1596, 1599, 1622; or a heavy chain with three CDRs comprising the amino acid sequences 1552, 1567, 1582 and a light chain with three CDRs comprising the amino acid sequences 1597, 1608, 1623; or a heavy chain with three CDRs comprising the amino acid sequences 1553, 1568, 1583 and a light chain with three CDRs comprising the amino acid sequences 1598, 1609 1624.

SARS (26)

Exemplary SARS neutralizing antibodies include antibodies having a _{VH} nucleotide sequence having SEQ ID NO: 1626 and a _{VL} nucleotide sequence having SEQ ID NO: 1628; a VH nucleotide sequence having SEQ ID NO: 1630 and a VL nucleotide sequence having SEQ ID NO: 1639; a VH nucleotide sequence having SEQ ID NO: 1634 and a VL nucleotide sequence having SEQ ID NO: 1640; a VH nucleotide sequence having SEQ ID NO: 1632 and a VL nucleotide sequence having SEQ ID NO: 1641; a VH nucleotide sequence having SEQ ID NO: 1633 and a VL nucleotide sequence having SEQ ID NO: 1642; a VH nucleotide sequence having SEQ ID NO: 1634 and a VL nucleotide sequence having SEQ ID NO: 1643; a VH nucleotide sequence having SEQ ID NO: 1635 and a VL nucleotide sequence having SEQ ID NO: 1644; a VH nucleotide sequence having SEQ ID NO: 1636 and a VL nucleotide sequence having SEQ ID NO: 1645; a VH nucleotide sequence having SEQ ID NO: 1637 and a VL nucleotide sequence having SEQ ID NO: 1646

CXCR4 (33)

Exemplary anti-CXCR4 antibody include antibodies having a VH amino acid sequence having SEQ ID NO: 771 and a VL amino acid sequence having SEQ ID NO: 779; a VH amino acid sequence having SEQ ID NO: 772 and a VL amino acid sequence having SEQ ID NO: 780; a VH amino acid sequence having SEQ ID NO: 773 and a VL amino acid sequence having SEQ ID NO: 781; a VH amino acid sequence having SEQ ID NO: 774 and a VL amino acid sequence having SEQ ID NO: 782; a VH amino acid sequence having SEQ ID NO: 775 and a VL amino acid sequence having SEQ ID NO: 783; a VH amino acid sequence having SEQ ID NO: 776 and a VL amino acid sequence having SEQ ID NO: 784; a VH amino acid sequence having SEQ ID NO: 777 and a VL amino acid sequence having SEQ ID NO: 785; or a VH amino acid sequence having SEQ ID NO: 778 and a VL amino acid sequence having SEQ ID NO: 786.

In other embodiments the anti-CXCR4 antibodies have a heavy chain with three CDRs including the amino acid sequences SEQ ID NO: 803, 804, 805 respectively and a light chain with three CDRs including the amino acid sequences 806, 807, 808 respectively; or a heavy chain with three CDRs comprising the amino acid sequences 809, 810, 811, respectively and a light chain with three CDRs comprising the amino acid sequences 812, 813, 814, respectively; or a heavy chain with three CDRs comprising the amino acid sequences 815, 816, 817 respectively and a light chain with three CDRs comprising the amino acid sequences 818, 819, 820 respectively; or a heavy chain with three CDRs comprising the amino acid sequences 827, 828, 829 respectively and a light chain with three CDRs comprising the amino acid sequences 830, 831, 832 respectively; or a heavy chain with three CDRs comprising the amino acid sequences 833, 834, 835, respectively and a light chain with three CDRs comprising the amino acid sequences 836, 837, 838, respectively; or a heavy chain with three CDRs comprising the amino acid sequences 839, 840, 841 respectively and a light chain with three CDRs comprising the amino acid sequences 842, 843, 844 respectively.

Carbonic Anhydrase IX (40)

Exemplary anti-CA IX antibodies include antibodies having a _{VH} amino acid sequence having SEQ ID NO: 845 and a _{VL} amino acid sequence having SEQ ID NO: 846; a VH amino acid sequence having SEQ ID NO: 847 and a VL amino acid sequence having SEQ ID NO: 868; a VH amino acid sequence having SEQ ID NO: 848 and a VL amino acid sequence having SEQ ID NO: 869; a VH amino acid sequence having SEQ ID NO: 849 and a VL amino acid sequence having SEQ ID NO: 870; a VH amino acid sequence having SEQ ID NO: 850 and a VL amino acid sequence having SEQ ID NO: 871; a VH amino acid sequence having SEQ ID NO: 851 and a VL amino acid sequence having SEQ ID NO: 872; a VH amino acid sequence having SEQ ID NO: 852 and a VL amino acid sequence having SEQ ID NO: 873; a VH amino acid sequence having SEQ ID NO: 853 and a VL amino acid sequence having SEQ ID NO: 874; a VH amino acid sequence having SEQ ID NO: 854 and a VL amino acid sequence having SEQ ID NO: 875; a VH amino acid sequence having SEQ ID NO: 855 and a VL amino acid sequence having SEQ ID NO: 876; a VH amino acid sequence having SEQ ID NO: 856 and a VL amino acid sequence having SEQ ID NO: 877; a VH amino acid sequence having SEQ ID NO: 857 and a VL amino acid sequence having SEQ ID NO: 878; a VH amino acid sequence having SEQ ID NO: 858 and a VL amino acid sequence having SEQ ID NO: 879; a VH amino acid sequence having SEQ ID NO: 859 and a VL amino acid sequence having SEQ ID NO: 880; a VH amino acid sequence having SEQ ID NO: 860 and a VL amino acid sequence having SEQ ID NO: 881; a VH amino acid sequence having SEQ ID NO: 861 and a VL amino acid sequence having SEQ ID NO: 882 a VH amino acid sequence having SEQ ID NO: 862 and a VL amino acid sequence having SEQ ID NO: 883; a VH amino acid sequence having SEQ ID NO: 863 and a VL amino acid sequence having SEQ ID NO: 884; a VH amino acid sequence having SEQ ID NO: 864 and a VL amino acid sequence having SEQ ID NO: 885; a VH amino acid sequence having SEQ ID NO: 865 and a VL amino acid sequence having SEQ ID NO: 886; a VH amino acid sequence having SEQ ID NO: 866 and a VL amino acid sequence having SEQ ID NO: 887; a VH amino acid sequence having SEQ ID NO: 867 and a VL amino acid sequence having SEQ ID NO: 888.

In other embodiments the anti-CA IX antibodies have a heavy chain with three CDRs including the amino acid sequences SEQ ID NO: 803, 804, 805 respectively and a light chain with three CDRs including the amino acid sequences 806, 807, 808 respectively; or a heavy chain with three CDRs comprising the amino acid sequences 899, 915, 909 and a light chain with three CDRs comprising the amino acid sequences 905, 906, 952 or a heavy chain with three CDRs comprising the amino acid sequences 899, 915, 909 and a light chain with three CDRs comprising the amino acid sequences 935, 943, 953 or a heavy chain with three CDRs comprising the amino acid sequences 899, 915, 909 and a light chain with three CDRs comprising the amino acid sequences 935, 906, 954 or a heavy chain with three CDRs comprising the amino acid sequences 910, 916, 923 and a light chain with three CDRs comprising the amino acid sequences 936, 944, 955 or a heavy chain with three CDRs comprising the amino acid sequences 899, 915, 909 and a light chain with three CDRs comprising the amino acid sequences 936, 944, 956 or a heavy chain with three CDRs comprising the amino acid sequences 911, 917, 924 and a light chain with three CDRs comprising the amino acid sequences 937, 945, 957 or a heavy chain with three CDRs comprising the amino acid sequences 899, 915, 909 and a light chain with three CDRs comprising the amino acid sequences 935, 946, 958 or a heavy chain with three CDRs comprising the amino acid sequences 899, 915, 909 and a light chain with three CDRs comprising the amino acid sequences 938, 946, 959 or a heavy chain with three CDRs comprising the amino acid sequences 899, 915, 909 and a light chain with three CDRs comprising the amino acid sequences 905, 946, 960 or a heavy chain with three CDRs comprising the amino acid sequences 899, 918, 925 and a light chain with three CDRs comprising the amino acid sequences 937, 947, 955 or a heavy chain with three CDRs comprising the amino acid sequences 899, 918, 926 and a light chain with three CDRs comprising the amino acid sequences 937, 945, 957 or a heavy chain with three CDRs comprising the amino acid sequences 912, 919, 927 and a light chain with three CDRs comprising the amino acid sequences 937, 943, 961 or a heavy chain with three CDRs comprising the amino acid sequences 899, 918, 928 and a light chain with three CDRs comprising the amino acid sequences 937, 906, 960 or a heavy chain with three CDRs comprising the amino acid sequences 899, 918, 928 and a light chain with three CDRs comprising the amino acid sequences 937, 906, 960 or a heavy chain with three CDRs comprising the amino acid sequences 913, 920, 929 and a light chain with three CDRs comprising the amino acid sequences 939, 948, 962 or a heavy chain with three CDRs comprising the amino acid sequences 899, 918, 930 and a light chain with three CDRs comprising the amino acid sequences 935, 944, 955 or a heavy chain with three CDRs comprising the amino acid sequences 899, 921, 931 and a light chain with three CDRs comprising the amino acid sequences 935, 944, 955 or a heavy chain with three CDRs comprising the amino acid sequences 912, 919, 932 and a light chain with three CDRs comprising the amino acid sequences 940, 949, 963 or a heavy chain with three CDRs comprising the amino acid sequences 899, 915, 909 and a light chain with three CDRs comprising the amino acid sequences 935, 943, 960 or a heavy chain with three CDRs comprising the amino acid sequences 914, 922, 933 and a light chain with three CDRs comprising the amino acid sequences 941, 950, 964 or a heavy chain with three CDRs comprising the amino acid sequences 912, 918, 934 and a light chain with three CDRs comprising the amino acid sequences 942, 951, 965.

CC-Chemokine Receptor 4 (CCR4) (048)

Exemplary CC-chemokine receptor 4 (CCR4) antibodies include antibodies having a _{VH} nucleotide sequence having SEQ ID NO: 969 and a _{VL} nucleotide sequence having SEQ ID NO: 971; a _{VH} nucleotide sequence having SEQ ID NO: 969 and a V_{L} nucleotide sequence having SEQ ID NO: 972.

Exemplary CCR4 antibodies include antibodies having a _{VH} amino acid sequence having SEQ ID NO: 970 and a _{VL} amino acid sequence having SEQ ID NO: 971.

In other embodiments the CCR4 antibodies have a heavy chain with three CDRs including the amino acid sequences SEQ ID NO: 973, 974, 975 respectively and a light chain with three CDRs including the amino acid sequences 976, 977, 978 respectively.

Middle East Respiratory Syndrome Coronavirus (MERS-CoV). (85)

Exemplary anti-Middle East Respiratory Syndrome coronavirus (MERS-CoV) antibody include antibodies having a VH nucleotide sequence having SEQ ID NO: 677 and a VL nucleotide sequence having SEQ ID NO:679; a VH nucleotide sequence having SEQ ID NO: 681 and a VL nucleotide sequence having SEQ ID NO:683; a VH nucleotide sequence having SEQ ID NO: 685 and a VL nucleotide sequence having SEQ ID NO:687; a VH nucleotide sequence having SEQ ID NO: 689 and a VL nucleotide sequence having SEQ ID NO:692; a VH nucleotide sequence having SEQ ID NO: 693 and a VL nucleotide sequence having SEQ ID NO:695; a VH nucleotide sequence having SEQ ID NO: 697 and a VL nucleotide sequence having SEQ ID NO:699; and a VH nucleotide sequence having SEQ ID NO: 701 and a VL nucleotide sequence having SEQ ID NO:703.

Exemplary anti-Middle East Respiratory Syndrome coronavirus (MERS-CoV) antibody include antibodies having a VH amino acid sequence SEQ ID NO: 678 and a VL amino acid sequence having SEQ ID NO: 680; a VH amino acid sequence SEQ ID NO: 682 and a VL amino acid sequence having SEQ ID NO: 684; a VH amino acid sequence SEQ ID NO: 686 and a VL amino acid sequence having SEQ ID NO: 688; a VH amino acid sequence SEQ ID NO: 690 and a VL amino acid sequence having SEQ ID NO: 692; a VH amino acid sequence SEQ ID NO: 694 and a VL amino acid sequence having SEQ ID NO: 696; a VH amino acid sequence SEQ ID NO: 698 and a VL amino acid sequence having SEQ ID NO: 700; and a VH amino acid sequence SEQ ID NO: 702 and a VL amino acid sequence having SEQ ID NO: 704.

In other embodiments the anti-Middle East Respiratory Syndrome coronavirus (MERS-CoV) antibody has a heavy chain with three CDRs including the amino acid sequences of 705, 706, and 707 and a light chain with three CDRs including the amino acid sequences 722, 723, and 724; a heavy chain with three CDRs including the amino acid sequences of 708, 709, and 710 and a light chain with three CDRs including the amino acid sequences 725, 726, and 727; a heavy chain with three CDRs including the amino acid sequences of 711, 712, and 713 and a light chain with three CDRs including the amino acid sequences 728, 729, and 730; a heavy chain with three CDRs including the amino acid sequences of 711, 735, and 715 and a light chain with three CDRs including the amino acid sequences 731, 732, and 733; a heavy chain with three CDRs including the amino acid sequences of 711, 735, and 716 and a light chain with three CDRs including the amino acid sequences 737, 738, and 739; a heavy chain with three CDRs including the amino acid sequences of 717, 718, and 719 and a light chain with three CDRs including the amino acid sequences 736, 742, and 743; and a heavy chain with three CDRs including the amino acid sequences of 714, 720, and 721 and a light chain with three CDRs including the amino acid sequences 740, 729, and 741.

GITR (93)

Exemplary anti-human GITR antibody include antibodies having a VH nucleotide sequence having SEQ ID NO: 1361 and a VL nucleotide sequence having SEQ ID NO: 1363; a VH nucleotide sequence having SEQ ID NO: 1365 and a VL nucleotide sequence having SEQ ID NO:1367; a VH nucleotide sequence having SEQ ID NO: 1369 and a VL nucleotide sequence having SEQ ID NO: 1371; a VH nucleotide sequence having SEQ ID NO: 1381 and a VL nucleotide sequence having SEQ ID NO: 1375; a VH nucleotide sequence having SEQ ID NO: 1377 and a VL nucleotide sequence having SEQ ID NO: 1379; a VH nucleotide sequence having SEQ ID NO: 1381 and a VL nucleotide sequence having SEQ ID NO: 1383; a VH nucleotide sequence having SEQ ID NO: 1385 and a VL nucleotide sequence having SEQ ID NO: 1387; a VH nucleotide sequence having SEQ ID NO: 1389 and a VL nucleotide sequence having SEQ ID NO:1391; a VH nucleotide sequence having SEQ ID NO: 1393 and a VL nucleotide sequence having SEQ ID NO: 1395; a VH nucleotide sequence having SEQ ID NO: 1397 and a VL nucleotide sequence having SEQ ID NO: 1398; or a VH nucleotide sequence having SEQ ID NO: 1401 and a VL nucleotide sequence having SEQ ID NO: 1403.

Exemplary anti-human GITR antibody include antibodies having a VH amino acid sequence having SEQ ID NO: 1362 and a VL amino acid sequence having SEQ ID NO: 1364; a VH amino acid having SEQ ID NO: 1366 and a VL polypeptide sequence having SEQ ID NO:1368; a VH amino acid sequence having SEQ ID NO: 1371 and a VL amino acid sequence having SEQ ID NO: 1372; a VH amino acid sequence having SEQ ID NO: 1382 and a VL amino acid sequence having SEQ ID NO: 1376; a VH nucleotide sequence having SEQ ID NO: 1378 and a VL nucleotide sequence having SEQ ID NO: 1380; a VH amino acid having SEQ ID NO: 1382 and a VL polypeptide sequence having SEQ ID NO: 1384; a VH amino acid sequence having SEQ ID NO: 1386 and a VL amino acid sequence having SEQ ID NO: 1388; a VH amino acid sequence having SEQ ID NO: 1390 and a VL amino acid sequence having SEQ ID NO: 1392; a VH amino acid having SEQ ID NO: 1394 and a VL polypeptide sequence having SEQ ID NO: 1396; a VH amino acid sequence having SEQ ID NO: 1399 and a VL amino acid sequence having SEQ ID NO: 1400; or a VH amino acid sequence having SEQ ID NO: 1402 and a VL amino acid sequence having SEQ ID NO: 1404.

In other embodiments the anti-human GITR antibody has a heavy chain with three CDRs including the amino acid sequences 1405, 1406, and 1407 and a light chain with three CDRs including the amino acid sequences1408, 1409, and 1410 respectively; a heavy chain with three CDRs including the amino acid sequences 1411, 1412, and 1413 and a light chain with three CDRs including the amino acid sequences1414, 1415, and 1416 respectively; a heavy chain with three CDRs including the amino acid sequences 1417, 1418, and 1419 and a light chain with three CDRs including the amino acid sequences 1420, 1421, and 1422 respectively; a heavy chain with three CDRs including the amino acid sequences 1423, 1424, and 1425 and a light chain with three CDRs including the amino acid sequences 1426, 1427, and 1428 respectively; a heavy chain with three CDRs including the amino acid sequences 1429, 1430, and 1431 and a light chain with three CDRs including the amino acid sequences 1432, 1433, and 1434 respectively; a heavy chain with three CDRs including the amino acid sequences 1435, 1436, and 1437 and a light chain with three CDRs including the amino acid sequences 1438, 1439, and 1440 respectively; a heavy chain with three CDRs including the amino acid sequences 1441, 1442, and 1443 and a light chain with three CDRs including the amino acid sequences 1444, 1445, and 1446 respectively; a heavy chain with three CDRs including the amino acid sequences 1447, 1448, and 1449 and a light chain with three CDRs including the amino acid sequences 1450, 1451, and 1452 respectively; a heavy chain with three CDRs including the amino acid sequences 1453, 1454, and 1455 and a light chain with three CDRs including the amino acid sequences 1456, 1457, and 1458 respectively; a heavy chain with three CDRs including the amino acid sequences 1459, 1460, and 1461 and a light chain with three CDRs including the amino acid sequences 1462, 1463, and 1464 respectively; or a heavy chain with three CDRs including the amino acid sequences 1465, 1466, and 1467 and a light chain with three CDRs including the amino acid sequences 1468, 1469, and 1470 respectively.

Flavivirus (73)

Exemplary anti-West Nile virus envelope protein E (WNE) antibody include antibodies having a VH nucleotide sequence having a VH amino acid sequence having SEQ ID NO: 1224 and a VL amino acid sequence having SEQ ID NO: 1226.

Exemplary anti-West Nile virus envelope protein E (WNE) antibody include antibodies having a VH nucleotide sequence having SEQ ID NO: 1225 and a VL nucleotide sequence having SEQ ID NO: 1227.

In other embodiments the anti-West Nile virus envelope protein E (WNE) antibody has a heavy chain with three CDRs including the amino acid sequences 1244, 1245, and 1246 and a light chain with three CDRs including the amino acid sequences 1247, 1248, and 1249 respectively.

CCR4 (65)

Exemplary anti-CC-chemokine receptor 4 (CCR4) antibody include antibodies having a _{VH} nucleotide sequence having SEQ ID NO: 1329 and a _{VL} nucleotide sequence having SEQ ID NO: 1331; a _{VH} nucleotide sequence having SEQ ID NO: 1333 and a V_{L} nucleotide sequence having SEQ ID NO:1335; a _{VH} nucleotide sequence having SEQ ID NO: 1337 and a V_{L} nucleotide sequence having SEQ ID NO: 1192; a _{VH} nucleotide sequence having SEQ ID NO: 1341 and a _{VL} nucleotide sequence having SEQ ID NO: 1343; or a _{VH} nucleotide sequence having SEQ ID NO: 1357 and a _{VL} nucleotide sequence having SEQ ID NO:1359.

Exemplary anti-CC-chemokine receptor 4 (CCR4) antibody include antibodies having a V_{H} amino acid sequence having SEQ ID NO: 1330 and a V_{L} amino acid sequence having SEQ ID NO: 1332; a V_{H} amino acid sequence having SEQ ID NO: 1334 and a V_{L} amino acid sequence having SEQ ID NO: 1336; a V_{H} amino acid sequence having SEQ ID NO: 1338 and a V_{L} amino acid sequence having SEQ ID NO: 1340; a V_{H} amino acid sequence having SEQ ID NO: 1342 and a V_{L} amino acid sequence having SEQ ID NO: 1344; or a V_{H} amino acid sequence having SEQ ID NO: 1358 and a V_{L} amino acid sequence having SEQ ID NO: 1360.

In other embodiments the anti-CC-chemokine receptor 4 (CCR4) antibody has a heavy chain with three CDRs including the amino acid sequences 1203, 1208, and 1211 and a light chain with three CDRs including the amino acid sequences 1207, 1209, and 1216 respectively; or a heavy chain with three CDRs including the amino acid sequences 1204, 1208, and 1212 and a light chain with three CDRs including the amino acid sequences 1207, 1209, and 1217 respectively; or a heavy chain with three CDRs including the amino acid sequences 1204, 1208, and 1213 and a light chain with three CDRs including the amino acid sequences 1207, 1209, and 1217 respectively; or a heavy chain with three CDRs including the amino acid sequences 1205, 1208, and 1214 and a light chain with three CDRs including the amino acid sequences 1207, 1209, and 1218 respectively; or a heavy chain with three CDRs including the amino acid sequences 1206, 1208, and 1210 and a light chain with three CDRs including the amino acid sequences 1207, 1209, and 1220 respectively; or a heavy chain with three CDRs including the amino acid sequences 1202, 1208, and 1210 and a light chain with three CDRs including the amino acid sequences 1207, 1209, and 1219 respectively.

Human Immunoglobulin Heavy Chain Variable Region Germline Gene VH1-69 (57)

Exemplary anti-human immunoglobulin heavy chain variable region germline gene VH1-69 antibody include antibodies having a VH nucleotide sequence having SEQ ID NO: 1153 and a VL nucleotide sequence having SEQ ID NO: 1155; or a VH nucleotide sequence having SEQ ID NO: 1163 and a VL nucleotide sequence having SEQ ID NO:1155.

Exemplary anti-human immunoglobulin heavy chain variable region germline gene VH1-69 antibody include antibodies having a V_{H} amino acid sequence having SEQ ID NO: 1154 and a V_{L} amino acid sequence having SEQ ID NO: 1156; or a V_{H} amino acid sequence having SEQ ID NO: 1164 and a V_{L} amino acid sequence having SEQ ID NO: 1156.

In other embodiments the anti-human immunoglobulin heavy chain variable region germline gene VH1-69 antibody has a heavy chain with three CDRs including the amino acid sequences 1157, 1158, and 1159 and a light chain with three CDRs including the amino acid sequences 1160, 1161, and 1162 respectively.

Influenza (49)

Exemplary anti-influenza antibody include antibodies having a VH nucleotide sequence having SEQ ID NO: 981 and a VL nucleotide sequence having SEQ ID NO: 983; a VH nucleotide sequence having SEQ ID NO: 985 and a VL nucleotide sequence having SEQ ID NO: 989; a VH nucleotide sequence having SEQ ID NO: 987 and a VL nucleotide sequence having SEQ ID NO: 991; a VH nucleotide sequence having SEQ ID NO: 993 and a VL nucleotide sequence having SEQ ID NO: 997; a VH nucleotide sequence having SEQ ID NO: 995 and a VK nucleotide sequence having SEQ ID NO: 999; a VH nucleotide sequence having SEQ ID NO: 1001 and a VL nucleotide sequence having SEQ ID NO: 1005; a VH nucleotide sequence having SEQ ID NO: 1003 and a VL nucleotide sequence having SEQ ID NO: 1007; a VH nucleotide sequence having SEQ ID NO: 1009 and a VL nucleotide sequence having SEQ ID NO: 1011; a VH nucleotide sequence having SEQ ID NO: 1013 and a VL nucleotide sequence having SEQ ID NO: 1015; and a VH nucleotide sequence having SEQ ID NO: 1017 and a VK nucleotide sequence having SEQ ID NO: 1019; a VH nucleotide sequence having SEQ ID NO: 1020 and a VL nucleotide sequence having SEQ ID NO: 1022.

Exemplary anti-influenza antibody include antibodies having a VH amino acid sequence having SEQ ID NO: 982 and a VL amino acid sequence having SEQ ID NO: 984; a VH amino acid sequence having SEQ ID NO: 986 and a VL amino acid sequence having SEQ ID NO: 988; a VH amino acid sequence having SEQ ID NO: 986 and a VL amino acid sequence having SEQ ID NO: 990; a VH amino acid sequence having SEQ ID NO: 992 and a VL amino acid sequence having SEQ ID NO: 994; a VH amino acid sequence having SEQ ID NO: 992 and a VK amino acid sequence having SEQ ID NO: 996; a VH amino acid sequence having SEQ ID NO: 998 and a VL amino acid sequence having SEQ ID NO: 1000; a VH amino acid sequence having SEQ ID NO: 998 and a VL amino acid sequence having SEQ ID NO: 1002; a VH amino acid sequence having SEQ ID NO: 1004 and a VL amino acid sequence having SEQ ID NO: 1006; a VH amino acid sequence having SEQ ID NO: 1008 and a VL amino acid sequence having SEQ ID NO: 1010; a VH amino acid sequence having SEQ ID NO: 1012 and a VK amino acid sequence having SEQ ID NO: 1014; and a VH amino acid sequence having SEQ ID NO: 1016 and a VL amino acid sequence having SEQ ID NO: 1018.

In other embodiments the anti-influenza antibody has a heavy chain with three CDRs including the amino acid sequences of 1023, 1031, and 1039 and a light chain with three CDRs including the amino acid sequences 1047, 1059, and 1071; a heavy chain with three CDRs including the amino acid sequences of 1023, 1032, and 1040 and a light chain with three CDRs including the amino acid sequences 1048, 1060, and 1072; a heavy chain with three CDRs including the amino acid sequences of 1025, 1032, and 1040 and a light chain with three CDRs including the amino acid sequences 1057, 1069, and 1081; a heavy chain with three CDRs including the amino acid sequences of 1026, 1033, and 1041 and a light chain with three CDRs including the amino acid sequences 1049, 1061, and 1073; a heavy chain with three CDRs including the amino acid sequences of 1026, 1033, and 1041 and a light chain with three CDRs including the amino acid sequences 1054, 1066, and 1078; a heavy chain with three CDRs including the amino acid sequences of 1027, 1034, and 1042 and a light chain with three CDRs including the amino acid sequences 1050, 1062, and 1074; a heavy chain with three CDRs including the amino acid sequences of 1027, 1034, and 1042 and a light chain with three CDRs including the amino acid sequences 1056, 1068, and 1080; a heavy chain with three CDRs including the amino acid sequences of 1028, 1035, and 1043 and a light chain with three CDRs including the amino acid sequences 1051, 1063, and 1065; a heavy chain with three CDRs including the amino acid sequences of 1028, 1036, and 1044 and a light chain with three CDRs including the amino acid sequences 1052, 1064, and 1076; a heavy chain with three CDRs including the amino acid sequences of 1029, 1037, and 1045 and a light chain with three CDRs including the amino acid sequences 1053, 1065, and 1077; or a heavy chain with three CDRs including the amino acid sequences of 1030, 1038, and 1046 and a light chain with three CDRs including the amino acid sequences 1058, 1070, and 1082.

Influenza (78)

Exemplary anti-influenza antibodies include antibodies having a _{VH} nucleotide sequence having SEQ ID NO: 397 and a _{VL} nucleotide sequence having SEQ ID NO: 398; a vx nucleotide sequence having SEQ ID NO: 399 and a V_{L} nucleotide sequence having SEQ ID NO:400; a _{VH} nucleotide sequence having SEQ ID NO: 401 and a V_{L} nucleotide sequence having SEQ ID NO: 402; a _{VH} nucleotide sequence having SEQ ID NO: 403 and a _{VL} nucleotide sequence having SEQ ID NO: 404; or a _{VH} nucleotide sequence having SEQ ID NO: 405 and a _{VL} nucleotide sequence having SEQ ID NO:406; or a _{VH} nucleotide sequence having SEQ ID NO: 407 and a _{VL} nucleotide sequence having SEQ ID NO:408; or a _{VH} nucleotide sequence having SEQ ID NO: 409 and a _{VL} nucleotide sequence having SEQ ID NO:410; or a _{VH} nucleotide sequence having SEQ ID NO: 411 and a _{VL} nucleotide sequence having SEQ ID NO:412; or a _{VH} nucleotide sequence having SEQ ID NO: 413 and a _{VL} nucleotide sequence having SEQ ID NO:414; or a _{VH} nucleotide sequence having SEQ ID NO: 415 and a _{VL} nucleotide sequence having SEQ ID NO:416; or a _{VH} nucleotide sequence having SEQ ID NO: 417 and a _{VL} nucleotide sequence having SEQ ID NO:418; or a _{VH} nucleotide sequence having SEQ ID NO: 419 and a _{VL} nucleotide sequence having SEQ ID NO:420; or a _{VH} nucleotide sequence having SEQ ID NO: 421 and a _{VL} nucleotide sequence having SEQ ID NO:422; or a _{VH} nucleotide sequence having SEQ ID NO: 423 and a _{VL} nucleotide sequence having SEQ ID NO:424; or a _{VH} nucleotide sequence having SEQ ID NO: 425 and a _{VL} nucleotide sequence having SEQ ID NO:426; or a _{VH} nucleotide sequence having SEQ ID NO: 427 and a _{VL} nucleotide sequence having SEQ ID NO:428; or a _{VH} nucleotide sequence having SEQ ID NO: 429 and a _{VL} nucleotide sequence having SEQ ID NO:430; or a _{VH} nucleotide sequence having SEQ ID NO: 431 and a _{VL} nucleotide sequence having SEQ ID NO:432; or a _{VH} nucleotide sequence having SEQ ID NO: 433 and a _{VL} nucleotide sequence having SEQ ID NO:434; or a _{VH} nucleotide sequence having SEQ ID NO: 435 and a _{VL} nucleotide sequence having SEQ ID NO:436; or a _{VH} nucleotide sequence having SEQ ID NO: 437 and a _{VL} nucleotide sequence having SEQ ID NO:438; or a _{VH} nucleotide sequence having SEQ ID NO: 439 and a _{VL} nucleotide sequence having SEQ ID NO:440; or a _{VH} nucleotide sequence having SEQ ID NO: 441 and a _{VL} nucleotide sequence having SEQ ID NO:442; or a VH nucleotide sequence having SEQ ID NO: 541 and a VL nucleotide sequence having SEQ ID NO: 542; or a VH nucleotide sequence having SEQ ID NO: 543 and a VL nucleotide sequence having SEQ ID NO: 544; or a VH nucleotide sequence having SEQ ID NO: 545 and a VL nucleotide sequence having SEQ ID NO: 546; or a VH nucleotide sequence having SEQ ID NO: 547 and a VL nucleotide sequence having SEQ ID NO: 548; or a VH nucleotide sequence having SEQ ID NO: 549 and a VL nucleotide sequence having SEQ ID NO: 550; or a VH nucleotide sequence having SEQ ID NO: 551 and a VL nucleotide sequence having SEQ ID NO: 552; or a VH nucleotide sequence having SEQ ID NO: 553 and a VL nucleotide sequence having SEQ ID NO: 554; or a VH nucleotide sequence having SEQ ID NO: 555 and a VL nucleotide sequence having SEQ ID NO: 556; or a VH nucleotide sequence having SEQ ID NO: 557 and a VL nucleotide sequence having SEQ ID NO: 558; or a VH nucleotide sequence having SEQ ID NO: 559 and a VL nucleotide sequence having SEQ ID NO: 560; or a VH nucleotide sequence having SEQ ID NO: 561 and a VL nucleotide sequence having SEQ ID NO: 562; or a VH nucleotide sequence having SEQ ID NO: 563 and a VL nucleotide sequence having SEQ ID NO: 564; or a VH nucleotide sequence having SEQ ID NO: 565 and a VL nucleotide sequence having SEQ ID NO: 566; or a VH nucleotide sequence having SEQ ID NO: 567 and a VL nucleotide sequence having SEQ ID NO: 568; or a VH nucleotide sequence having SEQ ID NO: 569 and a VL nucleotide sequence having SEQ ID NO: 570; or a VH nucleotide sequence having SEQ ID NO: 571 and a VL nucleotide sequence having SEQ ID NO: 572; or a VH nucleotide sequence having SEQ ID NO: 573 and a VL nucleotide sequence having SEQ ID NO: 574; or a VH nucleotide sequence having SEQ ID NO: 575 and a VL nucleotide sequence having SEQ ID NO: 576; or a VH nucleotide sequence having SEQ ID NO: 577 and a VL nucleotide sequence having SEQ ID NO: 578; or a VH nucleotide sequence having SEQ ID NO: 579 and a VL nucleotide sequence having SEQ ID NO: 580; or a VH nucleotide sequence having SEQ ID NO: 581 and a VL nucleotide sequence having SEQ ID NO: 582; or a VH nucleotide sequence having SEQ ID NO: 583 and a VL nucleotide sequence having SEQ ID NO: 584; or a VH nucleotide sequence having SEQ ID NO: 585 and a VL nucleotide sequence having SEQ ID NO: 586; or a VH nucleotide sequence having SEQ ID NO: 587 and a VL nucleotide sequence having SEQ ID NO: 588; or a VH nucleotide sequence having SEQ ID NO: 589 and a VL nucleotide sequence having SEQ ID NO: 590; or a VH nucleotide sequence having SEQ ID NO: 591 and a VL nucleotide sequence having SEQ ID NO: 592; or a VH nucleotide sequence having SEQ ID NO: 593 and a VL nucleotide sequence having SEQ ID NO: 594; or a VH nucleotide sequence having SEQ ID NO: 595 and a VL nucleotide sequence having SEQ ID NO: 596; or a VH nucleotide sequence having SEQ ID NO: 597 and a VL nucleotide sequence having SEQ ID NO: 598; or a VH nucleotide sequence having SEQ ID NO: 599 and a VL nucleotide sequence having SEQ ID NO: 600.

Exemplary anti-influenza antibodies antibody include antibodies having a _{VH} amino acid sequence having SEQ ID NO: 469 and a _{VL} amino acid sequence having SEQ ID NO: 470; a _{VH} amino acid having SEQ ID NO: 471 and a V_{L} polypeptide sequence having SEQ ID NO:472; a _{VH} amino acid sequence having SEQ ID NO: 473 and a V_{L} amino acid sequence having SEQ ID NO: 474; a _{VH} amino acid sequence having SEQ ID NO: 475 and a _{VL} amino acid sequence having SEQ ID NO: 476; or a _{VH} nucleotide sequence having SEQ ID NO: 477 and a _{VL} nucleotide sequence having SEQ ID NO:478; a _{VH} amino acid sequence having SEQ ID NO: 479 and a _{VL} amino acid sequence having SEQ ID NO: 480; a _{VH} amino acid sequence having SEQ ID NO: 481 and a _{VL} amino acid sequence having SEQ ID NO: 482; a _{VH} amino acid sequence having SEQ ID NO: 483 and a _{VL} amino acid sequence having SEQ ID NO: 484; a _{VH} amino acid sequence having SEQ ID NO: 485 and a _{VL} amino acid sequence having SEQ ID NO: 486; a _{VH} amino acid sequence having SEQ ID NO: 487 and a _{VL} amino acid sequence having SEQ ID NO: 488; a _{VH} amino acid sequence having SEQ ID NO: 489 and a _{VL} amino acid sequence having SEQ ID NO: 490; a _{VH} amino acid sequence having SEQ ID NO: 491 and a _{VL} amino acid sequence having SEQ ID NO: 492; a _{VH} amino acid sequence having SEQ ID NO: 493 and a _{VL} amino acid sequence having SEQ ID NO: 494; a _{VH} amino acid sequence having SEQ ID NO: 495 and a _{VL} amino acid sequence having SEQ ID NO: 496; a _{VH} amino acid sequence having SEQ ID NO: 497 and a _{VL} amino acid sequence having SEQ ID NO: 498; a _{VH} amino acid sequence having SEQ ID NO: 499 and a _{VL} amino acid sequence having SEQ ID NO: 500; a _{VH} amino acid sequence having SEQ ID NO: 501 and a _{VL} amino acid sequence having SEQ ID NO: 502; a _{VH} amino acid sequence having SEQ ID NO: 503 and a _{VL} amino acid sequence having SEQ ID NO: 504; a _{VH} amino acid sequence having SEQ ID NO: 505 and a _{VL} amino acid sequence having SEQ ID NO: 506; a _{VH} amino acid sequence having SEQ ID NO: 507 and a _{VL} amino acid sequence having SEQ ID NO: 508; a _{VH} amino acid sequence having SEQ ID NO: 509 and a _{VL} amino acid sequence having SEQ ID NO: 510; a _{VH} amino acid sequence having SEQ ID NO: 511 and a _{VL} amino acid sequence having SEQ ID NO: 512; a _{VH} amino acid sequence having SEQ ID NO: 513 and a _{VL} amino acid sequence having SEQ ID NO: 514; a _{VH} amino acid sequence having SEQ ID NO: 515 and a _{VL} amino acid sequence having SEQ ID NO: 516; a _{VH} amino acid sequence having SEQ ID NO: 517 and a _{VL} amino acid sequence having SEQ ID NO: 518; a _{VH} amino acid sequence having SEQ ID NO: 519 and a _{VL} amino acid sequence having SEQ ID NO: 520; a _{VH} amino acid sequence having SEQ ID NO: 521 and a _{VL} amino acid sequence having SEQ ID NO: 522; a _{VH} amino acid sequence having SEQ ID NO: 523 and a _{VL} amino acid sequence having SEQ ID NO: 524; a _{VH} amino acid sequence having SEQ ID NO: 525 and a _{VL} amino acid sequence having SEQ ID NO: 526; a _{VH} amino acid sequence having SEQ ID NO: 527 and a _{VL} amino acid sequence having SEQ ID NO: 528; a _{VH} amino acid sequence having SEQ ID NO: 529 and a _{VL} amino acid sequence having SEQ ID NO: 530; a _{VH} amino acid sequence having SEQ ID NO: 531 and a _{VL} amino acid sequence having SEQ ID NO: 532; a _{VH} amino acid sequence having SEQ ID NO: 533 and a _{VL} amino acid sequence having SEQ ID NO: 534; a _{VH} amino acid sequence having SEQ ID NO: 535 and a _{VL} amino acid sequence having SEQ ID NO: 536; a _{VH} amino acid sequence having SEQ ID NO: 537 and a _{VL} amino acid sequence having SEQ ID NO: 538; a _{VH} amino acid sequence having SEQ ID NO: 539 and a _{VL} amino acid sequence having SEQ ID NO: 540 a VH amino acid sequence having SEQ ID NO: 601 and a VL amino acid sequence having SEQ ID NO: 602 a VH amino acid sequence having SEQ ID NO: 603 and a VL amino acid sequence having SEQ ID NO: 604 a VH amino acid sequence having SEQ ID NO: 605 and a VL amino acid sequence having SEQ ID NO: 606 a VH amino acid sequence having SEQ ID NO: 607 and a VL amino acid sequence having SEQ ID NO: 608 a VH amino acid sequence having SEQ ID NO: 609 and a VL amino acid sequence having SEQ ID NO: 610 a VH amino acid sequence having SEQ ID NO: 611 and a VL amino acid sequence having SEQ ID NO: 612 a VH amino acid sequence having SEQ ID NO: 613 and a VL amino acid sequence having SEQ ID NO: 614 a VH amino acid sequence having SEQ ID NO: 615 and a VL amino acid sequence having SEQ ID NO: 616 a VH amino acid sequence having SEQ ID NO: 617 and a VL amino acid sequence having SEQ ID NO: 618 a VH amino acid sequence having SEQ ID NO: 619 and a VL amino acid sequence having SEQ ID NO: 620 a VH amino acid sequence having SEQ ID NO: 621 and a VL amino acid sequence having SEQ ID NO: 622 a VH amino acid sequence having SEQ ID NO: 623 and a VL amino acid sequence having SEQ ID NO: 624 a VH amino acid sequence having SEQ ID NO: 625 and a VL amino acid sequence having SEQ ID NO: 626 a VH amino acid sequence having SEQ ID NO: 627 and a VL amino acid sequence having SEQ ID NO: 628 a VH amino acid sequence having SEQ ID NO: 629 and a VL amino acid sequence having SEQ ID NO: 630 a VH amino acid sequence having SEQ ID NO: 631 and a VL amino acid sequence having SEQ ID NO: 632 a VH amino acid sequence having SEQ ID NO: 633 and a VL amino acid sequence having SEQ ID NO: 634 a VH amino acid sequence having SEQ ID NO: 635 and a VL amino acid sequence having SEQ ID NO: 636 a VH amino acid sequence having SEQ ID NO: 637 and a VL amino acid sequence having SEQ ID NO: 638 a VH amino acid sequence having SEQ ID NO: 639 and a VL amino acid sequence having SEQ ID NO: 640 a VH amino acid sequence having SEQ ID NO: 641 and a VL amino acid sequence having SEQ ID NO: 642 a VH amino acid sequence having SEQ ID NO: 643 and a VL amino acid sequence having SEQ ID NO: 644 a VH amino acid sequence having SEQ ID NO: 645 and a VL amino acid sequence having SEQ ID NO: 646 a VH amino acid sequence having SEQ ID NO: 647 and a VL amino acid sequence having SEQ ID NO: 648 a VH amino acid sequence having SEQ ID NO: 649 and a VL amino acid sequence having SEQ ID NO: 650 a VH amino acid sequence having SEQ ID NO: 651 and a VL amino acid sequence having SEQ ID NO: 652 a VH amino acid sequence having SEQ ID NO: 653 and a VL amino acid sequence having SEQ ID NO: 654 a VH amino acid sequence having SEQ ID NO: 655 and a VL amino acid sequence having SEQ ID NO: 656 a VH amino acid sequence having SEQ ID NO: 657 and a VL amino acid sequence having SEQ ID NO: 658 a VH amino acid sequence having SEQ ID NO: 659 and a VL amino acid sequence having SEQ ID NO: 660.

In other embodiments the anti-influenza antibodies antibody has a heavy chain with three CDRs including the amino acid sequences SEQ ID NO: 1, 37, 73 respectively and a light chain with three CDRs including the amino acid sequences 109, 145, 181 respectively; or a heavy chain with three CDRs comprising the amino acid sequences 2, 38, 74 respectively and a light chain with three CDRs comprising the amino acid sequences 110, 146, 182, respectively; or a heavy chain with three CDRs comprising the amino acid sequences 3, 39, 75 respectively and a light chain with three CDRs comprising the amino acid sequences 111, 147, 183, respectively; or a heavy chain with three CDRs comprising the amino acid sequences 4, 40, 76 respectively and a light chain with three CDRs comprising the amino acid sequences 112, 148, 184, respectively; or a heavy chain with three CDRs comprising the amino acid sequences 5, 41, 77 respectively and a light chain with three CDRs comprising the amino acid sequences 113, 149, 185, respectively; or a heavy chain with three CDRs comprising the amino acid sequences 6, 42, 78 respectively and a light chain with three CDRs comprising the amino acid sequences 114, 150, 186, respectively; or a heavy chain with three CDRs comprising the amino acid sequences 7, 43, 79 respectively and a light chain with three CDRs comprising the amino acid sequences 115, 151, 187, respectively; or a heavy chain with three CDRs comprising the amino acid sequences 8, 44, 80 respectively and a light chain with three CDRs comprising the amino acid sequences 116, 152, 188, respectively; or a heavy chain with three CDRs comprising the amino acid sequences 9, 45, 81 respectively and a light chain with three CDRs comprising the amino acid sequences 117, 153, 189, respectively; or a heavy chain with three CDRs comprising the amino acid sequences 10, 46, 82 respectively and a light chain with three CDRs comprising the amino acid sequences 118, 154, 190, respectively; or a heavy chain with three CDRs comprising the amino acid sequences 11, 47, 83 respectively and a light chain with three CDRs comprising the amino acid sequences 119, 155, 191, respectively; or a heavy chain with three CDRs comprising the amino acid sequences 12, 48, 84 respectively and a light chain with three CDRs comprising the amino acid sequences 120, 156, 192, respectively; or a heavy chain with three CDRs comprising the amino acid sequences 13, 49, 85 respectively and a light chain with three CDRs comprising the amino acid sequences 121, 157, 193, respectively; or a heavy chain with three CDRs comprising the amino acid sequences 14, 50, 86 respectively and a light chain with three CDRs comprising the amino acid sequences 122, 158, 194, respectively; or a heavy chain with three CDRs comprising the amino acid sequences 15, 51, 87 respectively and a light chain with three CDRs comprising the amino acid sequences 123, 159, 195, respectively; or a heavy chain with three CDRs comprising the amino acid sequences 16, 52, 88 respectively and a light chain with three CDRs comprising the amino acid sequences 124, 160, 196, respectively; or a heavy chain with three CDRs comprising the amino acid sequences 17, 53, 89 respectively and a light chain with three CDRs comprising the amino acid sequences 125, 161, 197, respectively; or a heavy chain with three CDRs comprising the amino acid sequences 18, 54, 90 respectively and a light chain with three CDRs comprising the amino acid sequences 126, 162, 198, respectively; or a heavy chain with three CDRs comprising the amino acid sequences 19, 55, 91 respectively and a light chain with three CDRs comprising the amino acid sequences 127, 163, 199, respectively; or a heavy chain with three CDRs comprising the amino acid sequences 20, 56, 92 respectively and a light chain with three CDRs comprising the amino acid sequences 128, 164, 200, respectively; or a heavy chain with three CDRs comprising the amino acid sequences 21, 57, 93 respectively and a light chain with three CDRs comprising the amino acid sequences 129, 165, 201, respectively; or a heavy chain with three CDRs comprising the amino acid sequences 22, 58, 94 respectively and a light chain with three CDRs comprising the amino acid sequences 130, 166, 202, respectively; or a heavy chain with three CDRs comprising the amino acid sequences 23, 59, 95 respectively and a light chain with three CDRs comprising the amino acid sequences 131, 167, 203, respectively; or a heavy chain with three CDRs comprising the amino acid sequences 24, 60, 96 respectively and a light chain with three CDRs comprising the amino acid sequences 132, 168, 204, respectively; or a heavy chain with three CDRs comprising the amino acid sequences 25, 61, 95 respectively and a light chain with three CDRs comprising the amino acid sequences 133, 169, 205, respectively; or a heavy chain with three CDRs comprising the amino acid sequences 26, 62, 96 respectively and a light chain with three CDRs comprising the amino acid sequences 134, 170, 206, respectively; or a heavy chain with three CDRs comprising the amino acid sequences 27, 63, 97 respectively and a light chain with three CDRs comprising the amino acid sequences 135, 171, 207, respectively; or a heavy chain with three CDRs comprising the amino acid sequences 28, 64, 98 respectively and a light chain with three CDRs comprising the amino acid sequences 136, 172, 208, respectively; or a heavy chain with three CDRs comprising the amino acid sequences 29, 65, 99 respectively and a light chain with three CDRs comprising the amino acid sequences 137, 173, 209, respectively; or a heavy chain with three CDRs comprising the amino acid sequences 30, 66, 100 respectively and a light chain with three CDRs comprising the amino acid sequences 138, 174, 210, respectively; or a heavy chain with three CDRs comprising the amino acid sequences 31, 67, 101 respectively and a light chain with three CDRs comprising the amino acid sequences 139, 175, 211, respectively; or a heavy chain with three CDRs comprising the amino acid sequences 32, 68, 102 respectively and a light chain with three CDRs comprising the amino acid sequences 140, 176, 212, respectively; or a heavy chain with three CDRs comprising the amino acid sequences 33, 69, 103 respectively and a light chain with three CDRs comprising the amino acid sequences 141, 177, 213, respectively; or a heavy chain with three CDRs comprising the amino acid sequences 34, 70, 104 respectively and a light chain with three CDRs comprising the amino acid sequences 142, 178, 214, respectively; or a heavy chain with three CDRs comprising the amino acid sequences 35, 71, 105 respectively and a light chain with three CDRs comprising the amino acid sequences 143, 179, 215, respectively; or a heavy chain with three CDRs comprising the amino acid sequences 36, 72, 106 respectively and a light chain with three CDRs comprising the amino acid sequences 144, 180, 216, respectively; or a heavy chain with three CDRs comprising the amino acid sequences 217, 247, 277 respectively and a light chain with three CDRs comprising the amino acid sequences 307, 337, 367, respectively; or a heavy chain with three CDRs comprising the amino acid sequences 218, 248, 278 respectively and a light chain with three CDRs comprising the amino acid sequences 308, 338, 368, respectively; or a heavy chain with three CDRs comprising the amino acid sequences 219, 249, 279 respectively and a light chain with three CDRs comprising the amino acid sequences 309, 339, 369, respectively; or a heavy chain with three CDRs comprising the amino acid sequences 220, 250, 280 respectively and a light chain with three CDRs comprising the amino acid sequences 310, 340, 370, respectively; or a heavy chain with three CDRs comprising the amino acid sequences 221, 251, 281 respectively and a light chain with three CDRs comprising the amino acid sequences 311, 341, 371, respectively; or a heavy chain with three CDRs comprising the amino acid sequences 222, 252, 282 respectively and a light chain with three CDRs comprising the amino acid sequences 312, 342, 372, respectively; or a heavy chain with three CDRs comprising the amino acid sequences 223, 253, 283 respectively and a light chain with three CDRs comprising the amino acid sequences 313, 343, 373, respectively; or a heavy chain with three CDRs comprising the amino acid sequences 224, 254, 284 respectively and a light chain with three CDRs comprising the amino acid sequences 314, 344, 374, respectively; or a heavy chain with three CDRs comprising the amino acid sequences 225, 255, 285 respectively and a light chain with three CDRs comprising the amino acid sequences 315, 345, 375, respectively; or a heavy chain with three CDRs comprising the amino acid sequences 226, 256, 286 respectively and a light chain with three CDRs comprising the amino acid sequences 316, 346, 376, respectively; or a heavy chain with three CDRs comprising the amino acid sequences 227, 257, 287 respectively and a light chain with three CDRs comprising the amino acid sequences 317, 347, 377, respectively; or a heavy chain with three CDRs comprising the amino acid sequences 228, 258, 288 respectively and a light chain with three CDRs comprising the amino acid sequences 318, 348, 378, respectively; or a heavy chain with three CDRs comprising the amino acid sequences 229, 259, 289 respectively and a light chain with three CDRs comprising the amino acid sequences 319, 349, 379, respectively; or a heavy chain with three CDRs comprising the amino acid sequences 230, 260, 290 respectively and a light chain with three CDRs comprising the amino acid sequences 320, 350, 380, respectively; or a heavy chain with three CDRs comprising the amino acid sequences 231, 261, 291 respectively and a light chain with three CDRs comprising the amino acid sequences 321, 351, 381, respectively; or a heavy chain with three CDRs comprising the amino acid sequences 232, 262, 292 respectively and a light chain with three CDRs comprising the amino acid sequences 322, 352, 382, respectively; or a heavy chain with three CDRs comprising the amino acid sequences 233, 263, 293 respectively and a light chain with three CDRs comprising the amino acid sequences 323, 353, 383, respectively; or a heavy chain with three CDRs comprising the amino acid sequences 234, 273, 294 respectively and a light chain with three CDRs comprising the amino acid sequences 324, 354, 384, respectively; or a heavy chain with three CDRs comprising the amino acid sequences 235, 274, 295 respectively and a light chain with three CDRs comprising the amino acid sequences 325, 355, 385, respectively; or a heavy chain with three CDRs comprising the amino acid sequences 236, 275, 296 respectively and a light chain with three CDRs comprising the amino acid sequences 326, 356, 386, respectively; or a heavy chain with three CDRs comprising the amino acid sequences 237, 276, 297 respectively and a light chain with three CDRs comprising the amino acid sequences 327, 357, 387, respectively; or a heavy chain with three CDRs comprising the amino acid sequences 237, 277, 298 respectively and a light chain with three CDRs comprising the amino acid sequences 328, 358, 388, respectively; or a heavy chain with three CDRs comprising the amino acid sequences 238, 278, 299 respectively and a light chain with three CDRs comprising the amino acid sequences 329, 359, 389, respectively; or a heavy chain with three CDRs comprising the amino acid sequences 239, 279, 300 respectively and a light chain with three CDRs comprising the amino acid sequences 330, 360, 390, respectively; or a heavy chain with three CDRs comprising the amino acid sequences 240, 280, 301 respectively and a light chain with three CDRs comprising the amino acid sequences 331, 361, 391, respectively; or a heavy chain with three CDRs comprising the amino acid sequences 241, 281, 302 respectively and a light chain with three CDRs comprising the amino acid sequences 332, 362, 392, respectively; or a heavy chain with three CDRs comprising the amino acid sequences 242, 282, 303 respectively and a light chain with three CDRs comprising the amino acid sequences 333, 363, 393, respectively; or a heavy chain with three CDRs comprising the amino acid sequences 243, 283, 304 respectively and a light chain with three CDRs comprising the amino acid sequences 334, 364, 394, respectively; or a heavy chain with three CDRs comprising the amino acid sequences 244, 284, 305 respectively and a light chain with three CDRs comprising the amino acid sequences 335, 365, 395, respectively; or a heavy chain with three CDRs comprising the amino acid sequences 245, 285, 306 respectively and a light chain with three CDRs comprising the amino acid sequences 336, 366, 396, respectively.

Other anti-influenza antibodies include those having the amino acid or nucleic acid sequences shown in the below Table 1.

TABLE 1

A. Antibody 3I14Variable Region nucleic
acid sequences
V_{H} chain of 3I14
(SEQ ID NO: 1665)
CAGGTGCAGCTGTTGGAGTCTGGGGGAGGCGTGGTCCAGCCTGGGA
GGTCCCTGAGACTCTCCTGTGCAGCCTCTGGATTCACCTTCAGTAA
CTATGGCATGCACTGGGTCCGCCAGGCTCCAGGCAAGGGGCTGGAG
TGGGTGGCAATTATATCATTTGATGGAAGTAAAAAATATTATGCAA
ACTCCGTGAAGGGCCGATCCACCATCTCCAGAGACAATTCCAAGAA
CACGCTGTCTCTGCAAATGAACAGCCTGGGACCTGAGGACACGGCT
CTATATTACTGTGCGAAACTGCCCTCCCCGTATTACTTTGATAGTC
GGTTCGTGTGGGTCGCCGCCAGCGCATTTCACTTCTGGGGCCAGGG
AATCCTGGTCACCGTCTCTTCA
V_{L} chain of 3I14
(SEQ ID NO: 1667)
AATTTTATGCTGACTCAGCCACCCTCAGCGTCTGGGACCCCCGGGC
AGAGGGTCACCATCTCTTGCTCTGGAAGCAGCTCCAACATCGGAGG
TAATACTGTACACTGGTTCCAGCAGCTCCCAGGAACGGCCCCCAAA
CTCCTCATCTATACTAATAGTCTGCGGCCCTCAGGGGTCCCTGACC
GATTCTCTGGCTCCAAGTCTGGCACCTCAGCCTCCCTGGCCATCAG
TGGGCTCCAGTCTGAGGATGAGGCTGATTATTACTGTGCAGCATGG
GATGACAGCCTAAATGGTCAGGTGTTCGGCGGAGGGACCAAGCTGA
CCGTCCTA
B. Antibody3I14Variable Region amino acid
sequences
V_{H} chain of 3I14
(SEQ ID NO: 1666)
QVQLLESGGGVVQPGRSLRLSCAASGFTFSNYGMHWVRQAPGKGLE
WVAIISFDGSKKYYANSVKGRSTISRDNSKNTLSLQMNSLGPEDTA
LYYCAKLPSPYYFDSRFVWVAASAFHFWGQGILVTVSS
V_{L} chain of 3I14
(SEQ ID NO: 1668)
NFMLTQPPSASGTPGQRVTISCSGSSSNIGGNTVHWFQQLPGTAPK
LLIYTNSLRPSGVPDRFSGSKSGTSASLAISGLQSEDEADYYCAAW
DDSLNGQVFGGGTKLTVL
C. Antibody 3I14V_{L}D94N Variable Region nucleic
acid sequence
V_{L} chain of 3I14V_{L}D94N
(SEQ ID NO: 1669)
AATTTTATGCTGACTCAGCCACCCTCAGCGTCTGGGACCCCCGGGC
AGAGGGTCACCATCTCTTGCTCTGGAAGCAGCTCCAACATCGGAGG
TAATACTGTACACTGGTTCCAGCAGCTCCCAGGAACGGCCCCCAAA
CTCCTCATCTATACTAATAGTCTGCGGCCCTCAGGGGTCCCTGACC
GATTCTCTGGCTCCAAGTCTGGCACCTCAGCCTCCCTGGCCATCAG
TGGGCTCCAGTCTGAGGATGAGGCTGATTATTACTGTGCAGCATGG
GATAACAGCCTAAATGGTCAGGTGTTCGGCGGAGGGACCAAGCTGA
CCGTCCTA
C. Antibody 3I14V_{L}D94N Variable Region amino
acid sequence
V_{L} chain of 3I14V_{L}D94N
(SEQ ID NO: 1670)
NFMLTQPPSASGTPGQRVTISCSGSSSNIGGNTVHWFQQLPGTAPK
LLIYTNSLRPSGVPDRFSGSKSGTSASLAISGLQSEDEADYYCAAW
DNSLNGQVFGGGTKLTVL

The amino acid sequences of the heavy and light chain complementary determining regions of the 3I14 and 3I14V_{L}D94N neutralizing influenza antibodies are shown in the below Table 2

TABLE 2

| | | |
|---|---|---|
| HCDR1 | GFTFSNYG | (SEQ ID NO: 1671) |
| HCDR2 | ISFDGSKK | (SEQ ID NO: 1672) |
| HCDR3 | CAKLPSPYYFDSR | (SEQ ID NO: 1673) |
| | FVWVAASAFHFW | |
| LCDR1 | SSNIGGNT | (SEQ ID NO: 1674) |
| LCDR2 | TNS | (SEQ ID NO: 1675) |
| LCDR3 | CAAWDDSLNGQVF | (SEQ ID NO: 1676) |
| 3I14V_{L}D94N | CAAWDNSLNGQVF | (SEQ ID NO: 1677) |
| LCDR3 | | |

CC-Chemokine Receptor 4 CCR4 (94)

Exemplary anti-CCR4 antibodies include antibodies having a VH nucleotide sequence having SEQ ID NO: 1678 and a VL nucleotide sequence having SEQ ID NO: 1679; or a VH nucleotide sequence having SEQ ID NO: 1680 and a VL nucleotide sequence having SEQ ID NO: 1681; or a VH nucleotide sequence having SEQ ID NO: 1682 and a VL nucleotide sequence having SEQ ID NO: 1683; or a VH nucleotide sequence having SEQ ID NO: 1684 and a VL nucleotide sequence having SEQ ID NO: 1685; or a VH nucleotide sequence having SEQ ID NO: 1686 and a VL nucleotide sequence having SEQ ID NO: 1687; or a VH nucleotide sequence having SEQ ID NO: 1688 and a VL nucleotide sequence having SEQ ID NO: 1689.

Exemplary anti-CCR4 antibodies include antibodies having a VH amino acid sequence having SEQ ID NO: 1690 and a VL amino acid sequence having SEQ ID NO: 1691; or a VH amino acid sequence having SEQ ID NO: 1692 and a VL amino acid sequence having SEQ ID NO: 1693; or a VH amino acid sequence having SEQ ID NO: 1694 and a VL amino acid sequence having SEQ ID NO: 1695; or a VH amino acid sequence having SEQ ID NO: 1696 and a VL amino acid sequence having SEQ ID NO: 1697; or a VH amino acid sequence having SEQ ID NO: 1698 and a VL amino acid sequence having SEQ ID NO: 1699; or a VH amino acid sequence having SEQ ID NO: 1700 and a VL amino acid sequence having SEQ ID NO: 1701.

In other embodiments the anti-influenza antibodies have a heavy chain with three CDRs having the amino acid sequences of SEQ ID NO: 1702, 1703, 1704, respectively, and a light chain with three CDRs having the amino acid sequences of SEQ ID NO: 1705, 1706, 1707, respectively; or a heavy chain with three CDRs having the amino acid sequences of SEQ ID NO: 1708, 1709, 1710, respectively, and a light chain with three CDRs having the amino acid sequences of SEQ ID NO: 1711, 1712, 1713, respectively; or a heavy chain with three CDRs having the amino acid sequences of SEQ ID NO: 1714, 1715, 1716, respectively, and a light chain with three CDRs having the amino acid sequences of SEQ ID NO: 1717, 1718, 1719, respectively; or a heavy chain with three CDRs having the amino acid sequences of SEQ ID NO: 1720, 1721, 1722, respectively, and a light chain with three CDRs having the amino acid sequences of SEQ ID NO: 1723, 1724, 1725, respectively; or a heavy chain with three CDRs having the amino acid sequences of SEQ ID NO: 1726, 1727, 1728, respectively, and a light chain with three CDRs having the amino acid sequences of SEQ ID NO: 1729, 1730, 1731, respectively; or a heavy chain with three CDRs having the amino acid sequences of SEQ ID NO: 1732, 1733, 1734, respectively, and a light chain with three CDRs having the amino acid sequences of SEQ ID NO: 1735, 1736, 1737, respectively; or a heavy chain with three CDRs having the amino acid sequences of SEQ ID NO: 1738, 1739, 1740, respectively, and a light chain with three CDRs having the amino acid sequences of SEQ ID NO: 1741, 1742, 1743, respectively.

Human Immunoglobulin Heavy Chain Variable Region Germline Gene (VH1-69) (133)

Exemplary anti-human immunoglobulin heavy chain variable region germline gene VH1-69 antibodies include a VH nucleotide sequence having SEQ ID NO: 1744 and a VL nucleotide sequence having SEQ ID NO: 1745; or a VH nucleotide sequence having SEQ ID NO: 1748 and a VL nucleotide sequence having SEQ ID NO: 1749; or a VH nucleotide sequence having SEQ ID NO: 1752 and a VL nucleotide sequence having SEQ ID NO: 1753.

Exemplary anti-human immunoglobulin heavy chain variable region germline gene VH1-69 antibodies include a VH amino acid sequence having SEQ ID NO: 1746 and a VL amino acid sequence having SEQ ID NO: 1747; or a VH amino acid sequence having SEQ ID NO: 1750 and a VL amino acid sequence having SEQ ID NO: 1751; or a VH amino acid sequence having SEQ ID NO: 1754 and a VL amino acid sequence having SEQ ID NO: 1755.

In other embodiments the anti-human immunoglobulin heavy chain variable region germline gene VH1-69 antibodies have a heavy chain with three CDRs having the amino acid sequences of SEQ ID NO: 1756, 1757, 1758, respectively, and a light chain with three CDRs having the amino acid sequences of SEQ ID NO: 1759, 1760, 1761, respectively.

Zika Virus Antibodies (140)

Exemplary antibodies that target and neutralize zika virus include antibodies having a VH nucleotide sequence having SEQ ID NO: 1762 and a VL nucleotide sequence having SEQ ID NO: 1763.

Exemplary antibodies that target and neutralize zika virus include antibodies having a VH amino acid sequence having SEQ ID NO: 1764 and a VL amino acid sequence having SEQ ID NO: 1765.

In other embodiments, the antibodies that target and neutralize zika virus have a heavy chain with three CDRs having the amino acid sequences of SEQ ID NO: 1766, 1767, 1768, respectively, and a light chain with three CDRs having the amino acid sequences of SEQ ID NO: 1769, 1770, 1771, respectively.

Glucocorticoid-Induced Tumor Necrosis Factor Receptor (GITR) (141)

Exemplary anti-glucocorticoid-induced tumor necrosis factor receptor (GITR) antibodies include a VH nucleotide sequence having SEQ ID NO: 1772 and a VL nucleotide sequence having SEQ ID NO: 1773; or a VH nucleotide sequence having SEQ ID NO: 1774 and a VL nucleotide sequence having SEQ ID NO: 1775; or a VH nucleotide sequence having SEQ ID NO: 1776 and a VL nucleotide sequence having SEQ ID NO: 1777; or a VH nucleotide sequence having SEQ ID NO: 1778 and a VL nucleotide sequence having SEQ ID NO: 1779; or a VH nucleotide sequence having SEQ ID NO: 1780 and a VL nucleotide sequence having SEQ ID NO: 1781; or a VH nucleotide sequence having SEQ ID NO: 1782 and a VL nucleotide sequence having SEQ ID NO: 1783; or a VH nucleotide sequence having SEQ ID NO: 1784 and a VL nucleotide sequence having SEQ ID NO: 1785; or a VH nucleotide sequence having SEQ ID NO: 1786 and a VL nucleotide sequence having SEQ ID NO: 1787; or a VH nucleotide sequence having SEQ ID NO: 1788 and a VL nucleotide sequence having SEQ ID NO: 1789; or a VH nucleotide sequence having SEQ ID NO: 1790 and a VL nucleotide sequence having SEQ ID NO: 1791; or a VH nucleotide sequence having SEQ ID NO: 1792 and a VL nucleotide sequence having SEQ ID NO: 1793; or a VH nucleotide sequence having SEQ ID NO: 1794 and a VL nucleotide sequence having SEQ ID NO: 1795; or a VH nucleotide sequence having SEQ ID NO: 1796 and a VL nucleotide sequence having SEQ ID NO: 1797.

Exemplary anti-glucocorticoid-induced tumor necrosis factor receptor (GITR) antibodies include a VH amino acid sequence having SEQ ID NO: 1798 and a VL amino acid sequence having SEQ ID NO: 1799; or a VH amino acid sequence having SEQ ID NO: 1800 and a VL amino acid sequence having SEQ ID NO: 1801; or a VH amino acid sequence having SEQ ID NO: 1802 and a VL amino acid sequence having SEQ ID NO: 1803; or a VH amino acid sequence having SEQ ID NO: 1804 and a VL amino acid sequence having SEQ ID NO: 1805; or a VH amino acid sequence having SEQ ID NO: 1806 and a VL amino acid sequence having SEQ ID NO: 1807; or a VH amino acid sequence having SEQ ID NO: 1808 and a VL amino acid sequence having SEQ ID NO: 1809; or a VH amino acid sequence having SEQ ID NO: 1810 and a VL amino acid sequence having SEQ ID NO: 1811; or a VH amino acid sequence having SEQ ID NO: 1812 and a VL amino acid sequence having SEQ ID NO: 1813; or a VH amino acid sequence having SEQ ID NO: 1814 and a VL amino acid sequence having SEQ ID NO: 1815; or a VH amino acid sequence having SEQ ID NO: 1816 and a VL amino acid sequence having SEQ ID NO: 1817; or a VH amino acid sequence having SEQ ID NO: 1818 and a VL amino acid sequence having SEQ ID NO: 1819; or a VH amino acid sequence having SEQ ID NO: 1820 and a VL amino acid sequence having SEQ ID NO: 1821; or a VH amino acid sequence having SEQ ID NO: 1822 and a VL amino acid sequence having SEQ ID NO: 1823.

In other embodiments, anti-glucocorticoid-induced tumor necrosis factor receptor (GITR) antibodies have a heavy chain with three CDRs having the amino acid sequences of SEQ ID NO: 1824, 1825, 1826, respectively, and a light chain with three CDRs having the amino acid sequences of SEQ ID NO: 1827, 1828, 1829, respectively; or a heavy chain with three CDRs having the amino acid sequences of SEQ ID NO: 1830, 1831, 1832, respectively, and a light chain with three CDRs having the amino acid sequences of SEQ ID NO: 1833, 1834, 1835, respectively; or a heavy chain with three CDRs having the amino acid sequences of SEQ ID NO: 1836, 1837, 1838, respectively, and a light chain with three CDRs having the amino acid sequences of SEQ ID NO: 1839, 1840, 1841, respectively; or a heavy chain with three CDRs having the amino acid sequences of SEQ ID NO: 1842, 1843, 1844, respectively, and a light chain with three CDRs having the amino acid sequences of SEQ ID NO: 1845, 1846, 1847, respectively; or a heavy chain with three CDRs having the amino acid sequences of SEQ ID NO: 1848, 1849, 1850, respectively, and a light chain with three CDRs having the amino acid sequences of SEQ ID NO: 1851, 1852, 1853, respectively; or a heavy chain with three CDRs having the amino acid sequences of SEQ ID NO: 1854, 1855, 1856, respectively, and a light chain with three CDRs having the amino acid sequences of SEQ ID NO: 1857, 1858, 1859, respectively; or a heavy chain with three CDRs having the amino acid sequences of SEQ ID NO: 1860, 1861, 1862, respectively, and a light chain with three CDRs having the amino acid sequences of SEQ ID NO: 1863, 1864, 1865, respectively; or a heavy chain with three CDRs having the amino acid sequences of SEQ ID NO: 1866, 1867, 1868, respectively, and a light chain with three CDRs having the amino acid sequences of SEQ ID NO: 1869, 1870, 1871, respectively; or a heavy chain with three CDRs having the amino acid sequences of SEQ ID NO: 1872, 1873, 1874, respectively, and a light chain with three CDRs having the amino acid sequences of SEQ ID NO: 1875, 1876, 1877, respectively; or a heavy chain with three CDRs having the amino acid sequences of SEQ ID NO: 1878, 1879, 1880, respectively, and a light chain with three CDRs having the amino acid sequences of SEQ ID NO: 1881, 1882, 1883, respectively; or a heavy chain with three CDRs having the amino acid sequences of SEQ ID NO: 1884, 1885, 1886, respectively, and a light chain with three CDRs having the amino acid sequences of SEQ ID NO: 1887, 1888, 1889, respectively; or a heavy chain with three CDRs having the amino acid sequences of SEQ ID NO: 1890, 1891, 1892, respectively, and a light chain with three CDRs having the amino acid sequences of SEQ ID NO: 1893, 1894, 1895, respectively; or a heavy chain with three CDRs having the amino acid sequences of SEQ ID NO: 1896, 1897, 1898, respectively, and a light chain with three CDRs having the amino acid sequences of SEQ ID NO: 1899, 1900, 1901.

The tetravalent antibody is a dimer of a bispecific scFv fragment having a first binding site for a first antigen, a second binding site for a second antigen. The scFv is preferably a tandem scFv. The variable domains of the two binding sites are joined together via a linker domain. In preferred embodiments the linker domain includes an immunoglobulin hinge region amino acid sequence. The hinge region is an IgG1, an IgG2, an IgG3, or an IgG4 hinge region. Exemplary hinge region amino acids sequences include EPKSCDKTHTCPPCP (SEQ ID NO:1902); ERKCCVECPPCP (SEQ ID NO:1903); and ESKYGPPCPSCP (SEQ ID NO:1904).

In some embodiments the linker domain further includes at least a portion of an immunoglobulin Fc domain. The at least a portion of an immunoglobulin Fc domain is an IgG1, an IgG2, an IgG3, or an IgG4 Fc domain. The at least a portion of an immunoglobulin Fc domain is linked to the C-terminus of the hinge region. By at least a portion of an immunoglobulin Fc domain is meant for example, an immunoglobulin CH2 domain amino acid sequence, CH3 domain amino acid sequence, CH4 domain amino acid sequence or any combination thereof.

Inclusion of at least a portion of an immunoglobulin Fc domain (e.g. CH2 domain) provides a third functional binding site (i.e. Fc effector function) resulting in a trifunctional bispecific antibody. Accordingly, it can be desirable to modify the at least a portion of with respect to effector function, so as to enhance, e.g., the effectiveness of the tBsAb. For example, amino acids substitution, insertion or deletion can be introduced into the at least a portion of the immunoglobulin Fc domain to generate tBsAbs having improved internalization capability and/or increased complement mediated cell killing and antibody dependent cellular cytotoxicity (ADCC). Alternatively, the at least a portion of the immunoglobulin Fc domain is glycosylated as to improve the stability and solubility of the tBsAbs. For example, the at least a portion of the immunoglobulin Fc domain is glycosylated at the amino acid corresponding to asparagine at amino acid position 297. While glycosylation is important for stability defucosylation of the CH2 carbohydrate can also increase the binding affinity to FcγRs and lead to further enhancement of ADCC.

In certain embodiments, the tBsAbs of the invention may comprise an Fc variant comprising an amino acid substitution which alters the antigen-independent effector functions of the antibody, in particular the circulating half-life of the antibody. Such antibodies exhibit either increased or decreased binding to FcRn when compared to antibodies lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered antibody is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting antibody has toxic side effects when present in the circulation for prolonged periods. In one embodiment, an Fc domain having one or more amino acid substitutions within the “FcRn binding loop” of an Fc domain. The FcRn binding loop is comprised of amino acid residues 280-299 (according to EU numbering). Exemplary amino acid substitutions which altered FcRn binding activity are disclosed in International PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering). [000126] Preferably the at least a portion of the Fc domain is a CH2 domain amino acid sequence. An exemplary CH2 domain amino acid sequence includes APELLGGPDVFLF (SEQ ID NO: 1905).

In other aspects the immunoglobulin hinge region amino acid sequence or the immunoglobulin hinge region/Fc domain amino acid sequence is flanked by a flexible linker amino acid sequence. Flexible linker amino acid sequences include for example is (GGGS)_{X=1-6} (SEQ ID NO: 1906), (GGGGS)_{X=1-6} (SEQ ID NO: 1907), or GSAGSAAGSGEF (SEQ ID NO: 1908).

Increasing the linker by adding multiple repeats (e.g., four or more) will predominantly result in a monomeric scFv, thus it can increase the accessibility to an epitope. Length and composition of the linkers can be chosen to optimize the stability and functional activity and takes into account the topography of the epitope on the target protein.

Also included in the invention is a nucleic acid construct including nucleic acids molecules encoding: a light chain and a heavy chain variable region of an antibody specifically binding to a first antigen; a light chain and heavy chain variable region of an antibody specifically binding to a second antigen; and a linker domain.

In yet a further aspect the invention provides a genetically engineered cell which expresses and bears on the cell surface membrane tBsAbs of the invention. The cell is a T-cell, a B-cell, a follicular T-Cell or an NK cell. The T cell is CD4+ or CD8+. The cell is a mixed population of CD4+ and CD8 cells+. The cell is further engineered to express and secrete the tBsAb.

Vectors including the nucleic acid constructs according to the invention and host cell, e.g., a mammalian cell, expressing the vectors of the invention.

Chimeric Antigen Receptors

The tBsAb of the invention can be used to produce a chimeric antigen receptor (CAR). The CAR generally comprises at least one transmembrane polypeptide comprising at least one extracellular ligand-binding domain comprising the tBsAb of the invention and; one transmembrane polypeptide comprising at least one intracellular signaling domain.

In a preferred embodiment said transmembrane domain further comprises a stalk region between said extracellular ligand-binding domain and said transmembrane domain. The term “stalk region” used herein generally means any oligo- or polypeptide that functions to link the transmembrane domain to the extracellular ligand-binding domain. In particular, stalk region are used to provide more flexibility and accessibility for the extracellular ligand-binding domain. A stalk region may comprise up to 300 amino acids, preferably 10 to 100 amino acids and most preferably 25 to 50 amino acids. Stalk region may be derived from all or part of naturally occurring molecules, such as from all or part of the extracellular region of CD8, CD4 or CD28, or from all or part of an antibody constant region. Alternatively the stalk region may be a synthetic sequence that corresponds to a naturally occurring stalk sequence, or may be an entirely synthetic stalk sequence. In a preferred embodiment said stalk region is a part of human CD8 alpha chain

The signal transducing domain or intracellular signaling domain of the CAR of the invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector signal function signal and directs the cell to perform a specialized function.

Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non limiting examples those derived from TCR zeta, FcR gamma, FcR beta, FcR epsilon, CD3 gamma, CD3 delta, CD3 epsilon, CD5, CD22, CD79a, CD79b and CD66d. In a preferred embodiment, the signaling transducing domain of the CAR can comprise the CD3 zeta signaling domain, or the intracytoplasmic domain of the Fc epsilon RI beta or gamma chains. In another preferred embodiment, the signaling is provided by CD3 zeta together with co-stimulation provided by CD28 and/or a tumor necrosis factor receptor (TNFr), such as 4-1BB or OX40), for example.

In particular embodiment the intracellular signaling domain of the CAR of the present invention comprises a co-stimulatory signal molecule. In some embodiments the intracellular signaling domain contains 2, 3, 4 or more co-stimulatory molecules in tandem. A co-stimulatory molecule is a cell surface molecule other than an antigen receptor or their ligands that is required for an efficient immune response.

“Co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a T-cell that specifically binds with a co-stimulatory ligand, thereby mediating a co-stimulatory response by the cell, such as, but not limited to proliferation. Co-stimulatory molecules include, but are not limited to an MHC class 1 molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like. The In another particular embodiment, said signal transducing domain is a TNFR-associated Factor 2 (TRAF2) binding motifs, intracytoplasmic tail of costimulatory TNFR member family. Cytoplasmic tail of costimulatory TNFR family member contains TRAF2 binding motifs consisting of the major conserved motif (P/S/A)X(Q/E)E) or the minor motif (PXQXXD), wherein X is any amino acid. TRAF proteins are recruited to the intracellular tails of many TNFRs in response to receptor trimerization.

The distinguishing features of appropriate transmembrane polypeptides comprise the ability to be expressed at the surface of an immune cell, in particular lymphocyte cells or Natural killer (NK) cells, and to interact together for directing cellular response of immune cell against a predefined target cell. The different transmembrane polypeptides of the CAR of the present invention comprising an extracellular ligand-biding domain and/or a signal transducing domain interact together to take part in signal transduction following the binding with a target ligand and induce an immune response. The transmembrane domain can be derived either from a natural or from a synthetic source. The transmembrane domain can be derived from any membrane-bound or transmembrane protein although certain transmembrane domains that that best accommodate the chimeras are preferred, e.g., those that promote self-aggregation or promote an increase in CART cell basal activation in the absence of target binding, which could lead to premature exhaustion.

The term “a part of” used herein refers to any subset of the molecule, that is a shorter peptide. Alternatively, amino acid sequence functional variants of the polypeptide can be prepared by mutations in the DNA which encodes the polypeptide. Such variants or functional variants include, for example, deletions from, or insertions or substitutions of, residues within the amino acid sequence. Any combination of deletion, insertion, and substitution may also be made to arrive at the final construct, provided that the final construct possesses the desired activity, especially to exhibit a specific anti-target cellular immune activity. The functionality of the CAR of the invention within a host cell is detectable in an assay suitable for demonstrating the signaling potential of said CAR upon binding of a particular target. Such assays are available to the skilled person in the art. For example, this assay allows the detection of a signaling pathway, triggered upon binding of the target, such as an assay involving measurement of the increase of calcium ion release, intracellular tyrosine phosphorylation, inositol phosphate turnover, or interleukin (IL) 2, interferon .gamma., GM-CSF, IL-3, IL-4 production thus effected.

Methods of Use

The tBsAbs, the cells expressing the tBsAbs or the CARs according to the invention can be used for treating cancer, viral infections or autoimmune disorders in a patient in need thereof. In another embodiment, tBsAbs, the cells expressing the tBsAbs or the CARs according to the invention can be used in the manufacture of a medicament for treatment of a cancer, viral infections of autoimmune disorders, in a patient in need thereof.

Said treatment can be ameliorating, curative or prophylactic. It may be either part of an autologous immunotherapy or part of an allogenic immunotherapy treatment. By autologous, it is meant that cells, cell line or population of cells used for producing the tBsAbs or the cells expressing the tBsAbs are originating from the patient or from a Human Leucocyte Antigen (HLA) compatible donor. By allogeneic is meant that the cells or population of cells used for producing the tBsAbs or the cells expressing the tBsAbs are not originating from the patient but from a donor.

Treatment can be used to treat patients diagnosed with cancer, viral infection, autoimmune disorders or Graft versus Host Disease (GvHD). Cancers that may be treated include tumors that are not vascularized, or not yet substantially vascularized, as well as vascularized tumors. The cancers may comprise nonsolid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

It can be a treatment in combination with one or more therapies against cancer selected from the group of antibodies therapy, chemotherapy, cytokines therapy, dendritic cell therapy, gene therapy, hormone therapy, laser light therapy and radiation therapy.

In a further embodiment, the compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAM PATH.

Definitions

It is to be noted that the term “a” or “an” entity refers to one or more of that entity: for example, “a bispecific antibody,” is understood to represent one or more bispecific antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The tern “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptide is meant to encompass both purified and recombinant polypeptides.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the World Wide Web (www) ncbi.nlm.nih.gov/blast/Blast.cgi, last accessed on May 21, 2008. Biologically equivalent polynucleotides are those having the above-noted specified percent homology and encoding a polypeptide having the same or similar biological activity.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent sequence retains the activity (e.g., epitope-binding) or structure (e.g., salt-bridge) of the reference sequence.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²+ normally found in a cell.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, the term “detectable label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

A “tandem scFv” is composed of two scFvs connected through a short linker, which allows the free rotation of the two separate antigen binding units, thus resulting in a flexible structure.

The term antibody encompasses various broad classes of polypeptides that can be distinguished biochemically. Those skilled in the art will appreciate that heavy chains are classified as gamma, mu, alpha, delta, or epsilon with some subclasses among them (e.g., .gamma.1-.gamma.4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab)₂, Fd, Fvs, single-chain Fvs (scFv), single-chain antibodies, disulfide-linked Fvs (sdFv), fragments comprising either a V_{K} or V_{H} domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda. Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (V_{K}) and heavy (V_{H}) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region: the CH3 and CK domains actually comprise the carboxy-terminus of the heavy and light chain, respectively.

As indicated above, the variable region allows the antibody to selectively recognize and specifically bind epitopes on antigens. That is, the V_{K} domain and V_{H} domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the V_{H} and V_{K} chains i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a b-sheet conformation and the CDRs form loops which connect, and in some cases form part of, the .beta.-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H 1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-LI and includes approximately 7 residues. CDR-L3 begins at approximately the thirty third residue after the end of CDR-L2 (i.e., following a cysteine residue); includes approximately 7-11 residues and ends at the sequence For W-G-X-G, where X is any amino acid.

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG, molecule and a hinge region derived from an IgG₃ molecule. In another example, a heavy chain constant region can comprise a hinge region derived, in part, from an IgG, molecule and, in part, from an IgG₃ molecule. In another example, a heavy chain portion can comprise a chimeric hinge derived, in part, from an IgG, molecule and, in part, from an IgG₄ molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three dimensional configuration of the constant regions of the various immunoglobulin classes are well known. As used herein, the term “V_{H} domain” includes the amino terminal variable domain of an immunoglobulin heavy chain and the term “CH1 domain” includes the first (most amino terminal) constant region domain of an immunoglobulin heavy chain. The CH1 domain is adjacent to the V_{H} domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CK regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

As used herein, “percent humanization” is calculated by determining the number of framework amino acid differences (i.e., non-CDR difference) between the humanized domain and the germline domain, subtracting that number from the total number of amino acids, and then dividing that by the total number of amino acids and multiplying by 100.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody hinds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

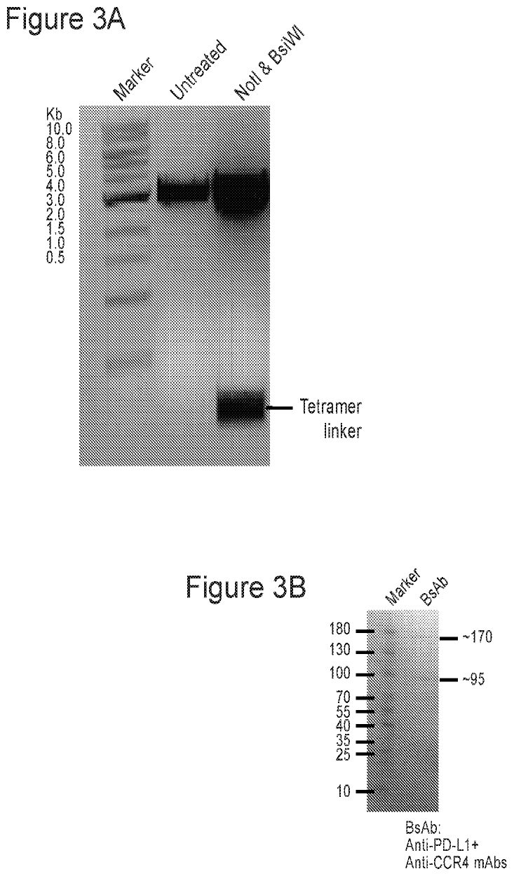

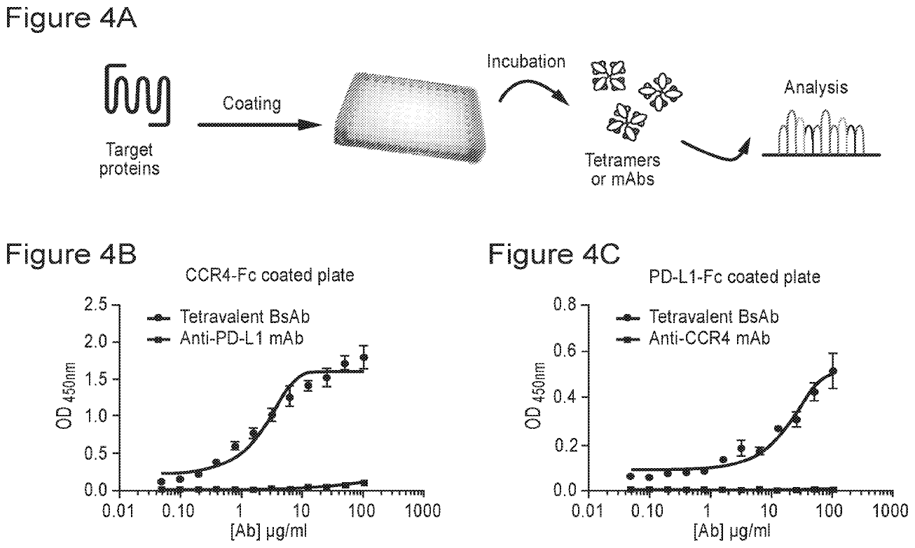

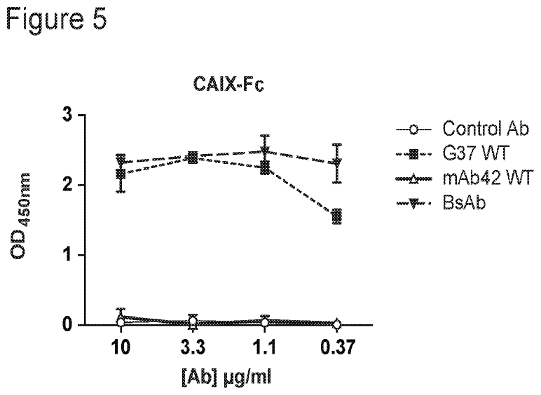

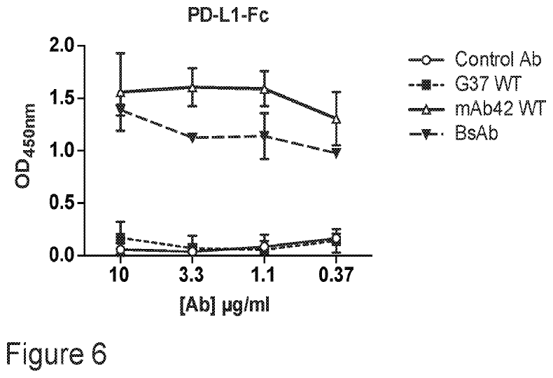

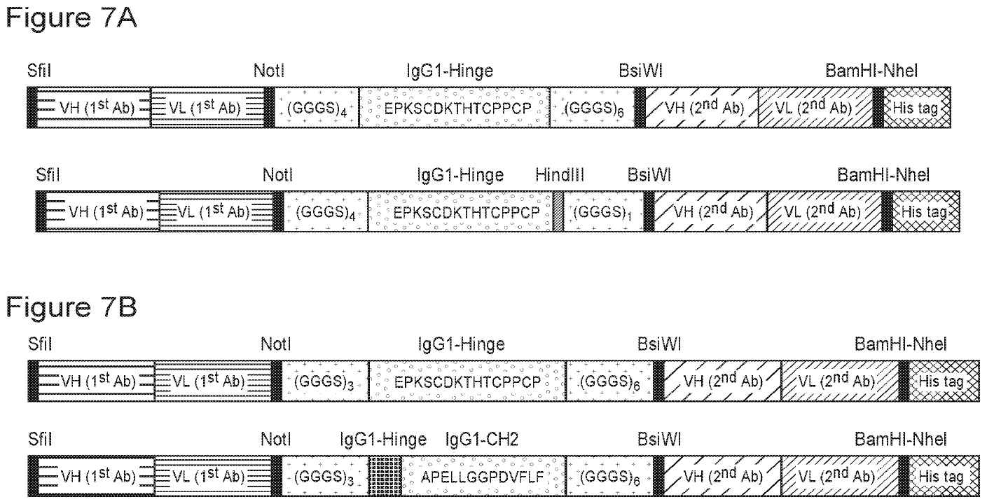

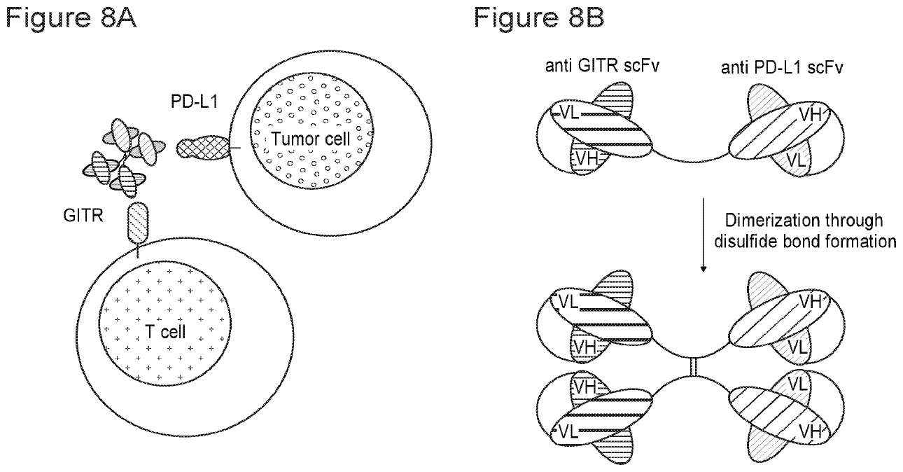

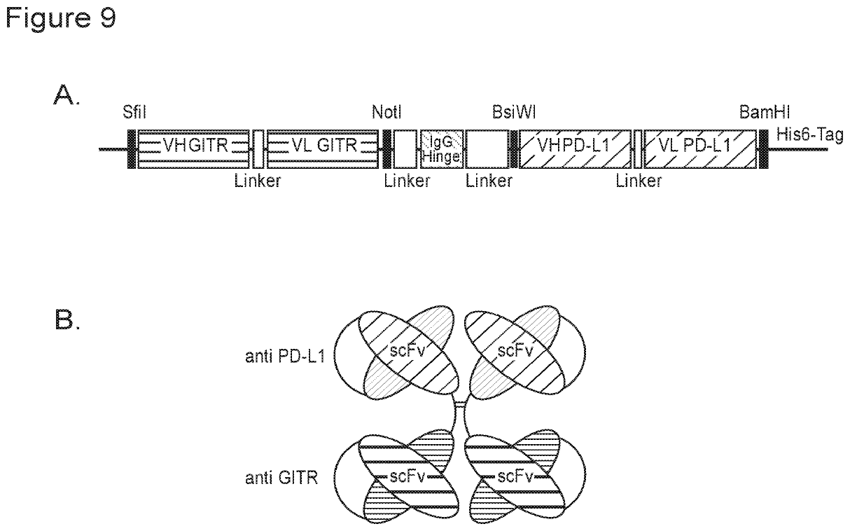

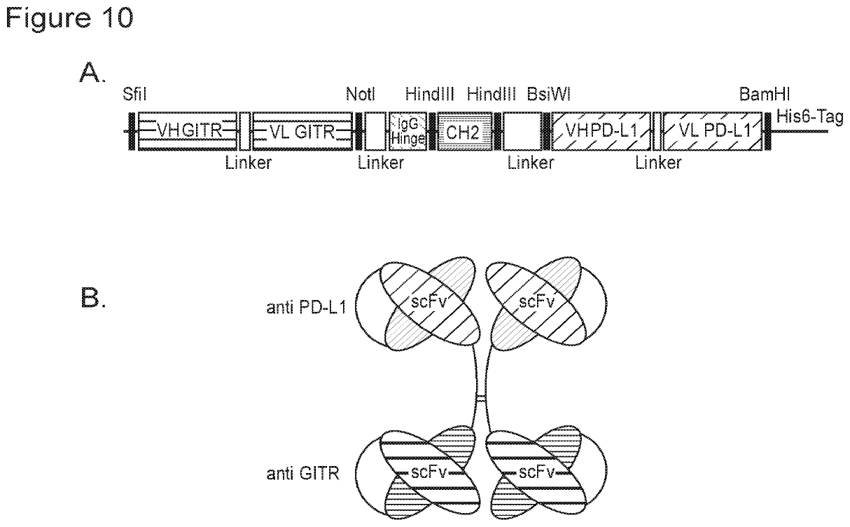

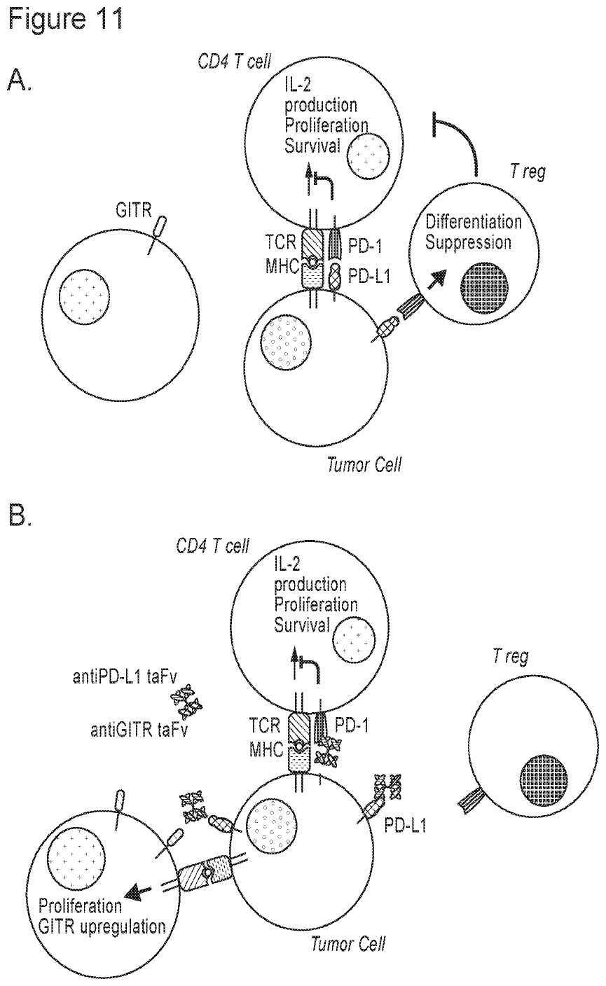

This disclosure describes the development of bispecific antibodies, which can be applied to cancer therapy. To accomplish this goal, two tetrameric bispecific antibodies (tBsAbs) with dual specificity for the GITR protein and the PD-L1 protein were created. An advantage of these constructs is that they will enhance anti-tumor response by activating T cells and abrogating regulatory T cell suppression. Also described are the anti-CCR4-anti-PDL1 tBsAb (FIGS. 3 and 4) and anti-CAIX-anti-PDL1 (FIGS. 5 and 6).

Here is described two different formats of tandem scFv fragment dimerization units in a pcDNA3.4 mammalian expression vector. In the first construct, the tandem scFvs comprise two scFvs derived from distinct parental antibodies. The αGITR scFv and αPD-L1 scFv are connected in tandem by an IgG1 hinge region between two flexible linkers. The first construct has a structure of VH GITR10-linker-VL GITR10-linker-hinge-linker-VH PD-L1-linker-VL PD-L1 and its sequence was confirmed by DNA sequencing. The second format was constructed equivalently to the first one. Yet, it includes an additional domain, e.g., a CH2 domain, introduced between the hinge and one linker region. The second construct has a structure of VH GITR-linker-VL GITR-linker-hinge-CH2-linker-VH PD-L1-linker-VL PD-L1. In contrast to the first construct, the second construct includes an Fc domain, potentially resulting in a trifunctional tBsAb.

Both tBsAbs were successfully expressed by transient transfection in HEK cells and purified by affinity chromatography using Ni-NTA agarose.

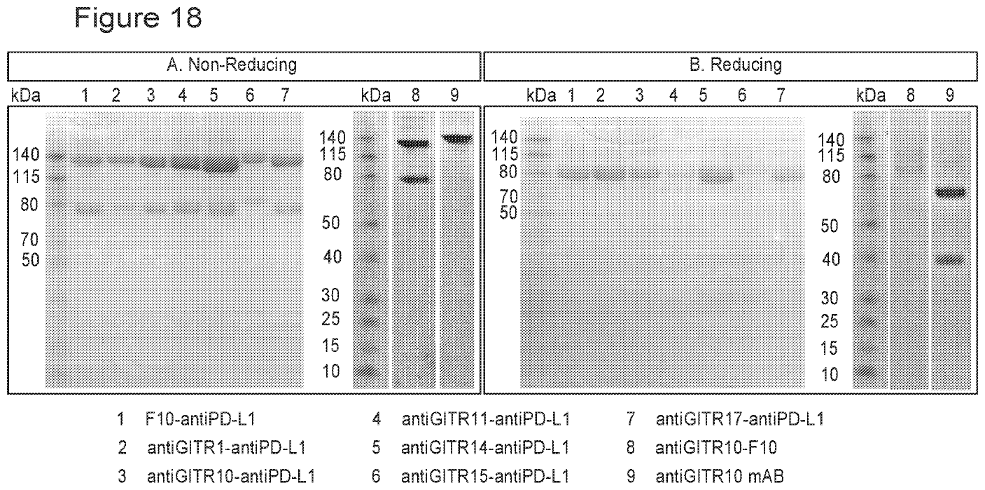

The purified proteins were evaluated by SDS-PAGE and results showed that under reducing conditions all protein profiles of the tBsAbs exhibit one single band and are congruent with the theoretical value of a tandem scFv (taFv): 65 kDa for αGITR-αPD-L1 taFv and 75 kDA for αGITR-αPD-L1 with CH2 taFv. Under non-reducing conditions the predicted molecular weight of αGITR-αPD-L1 (130 kDa) and αGITR-αPD-L1 with CH2 (150 kDa) tBsAbs match the apparent molecular weight (See, FIG. 18).

ELISA and flow cytometry demonstrated the biological activities of the newly designed tBsAbs. In ELISA the retained binding activity of the αPD-L1 arm of the produced BsAb was preserved in vitro and showed similar binding activity as the αPD-L1 mAb. Unspecific binding of the tBsAbs was to be ruled out since CCR4, to which the αGITR-αPD-L1 antibodies do not bind, did not show any signal. Furthermore, similar binding activity of the αGITR arm for both produced BsAb (αGITR-αPD-L1 and αGITR-αPD-L1 with CH2) to the GITR protein was observed. Unspecific binding was likewise excluded from this arm since the αGITR-αPD-L1 antibodies did not show any binding specificity for GITR-CF2 cells. When comparing the αGITR IgG to each BsAb, the αGITR IgG showed higher binding in the ELISA experiment, suggesting a lower affinity of the tBsAbs. Nevertheless depending on the spatial arrangement of the antigen binding sites as well as the antigen surface distribution bivalency of the tBsAbs can increase avidity, which can compensate for weak binding.

The flow cytometry analyses of αGITR-αPD-L1 tested with GITR+CF2 cells suggests that the novel tBsAb recognize the GITR protein in its native conformation when expressed on cells. A similar binding affinity of the αGITR-αPD-L1 tBsAbcompared to the GITR mAb was observed. The ELISA and Flow cytometry analyses therefore demonstrated the capability of the αGITR10-αPD-L1 and αGITR10-αPD-L1 with CH2 to specifically recognize the corresponding antigen when expressed on cells, as would be the case in vivo. These characterization studies exhibited similar binding behavior of the αGITR-αPD-L1 and αGITR-αPD-L1 with CH2.

An important aspect of the αGITR10-αPD-L1 with CH2 lays in its function in inducing complement-dependent cytotoxicity (CDC) and antibody-dependent cellular cytotoxicity (ADCC). These functions further the beneficial effects of the tBsAb when targeting tumor cells for destruction.

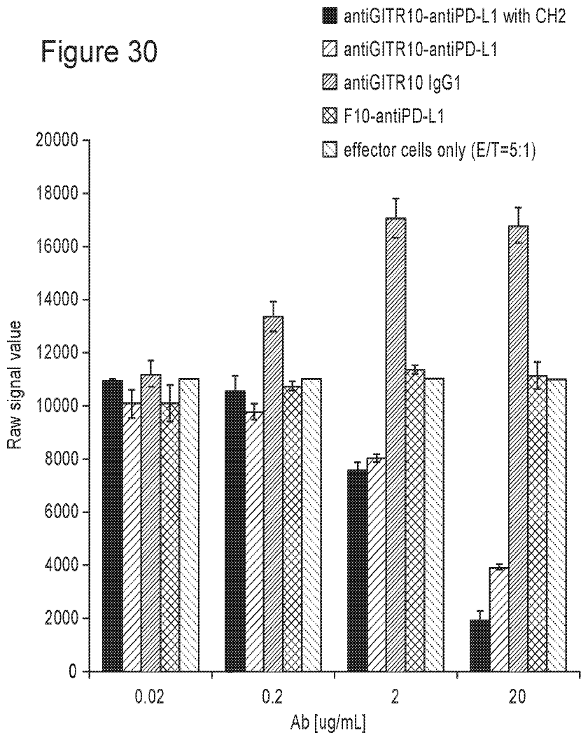

In the ADCC analysis the αGITR10-αPD-L1 and αGITR10-αPD-L1 with CH2 displayed surprising results using GITR+CF2 as target cells and WIL2-S as effector cells (E/T=5:1). For αGITR10-αPD-L1 and αGITR10-αPD-L1 with CH2 antibodies the raw signal of the luciferase activity decreased with higher antibody concentrations and was substantially lower than the signal for sole target and effector cells (See FIG. 30).

The herein-described methods allow generation of tBsAb involving only one cloning step. The tBsAb preserves the dual affinity towards the GITR protein and PD-L1 antigen in a small-sized molecule of only about 150 kDa. Such tBsAbs will be critical for effective cancer therapeutics.

### EXAMPLES

### Example 1: Cloning of αGITR-αPD-L1 Tetrameric Bispecific Antibody (tBsAb)

Cloning Strategy

The goal was to clone a plasmid that contained two recombinant single chain variable fragments (scFvs), originating from different parental antibodies and joined by a flexible linker. While one of the scFvs is directed against the GITR protein, the other is directed against PD-L1. Such a plasmid will produce two scFvs that are covalently joined by a linker-hinge-linker domain and result in a tetrameric bispecific antibody (αGITR-αPD-L1 tBsAb).

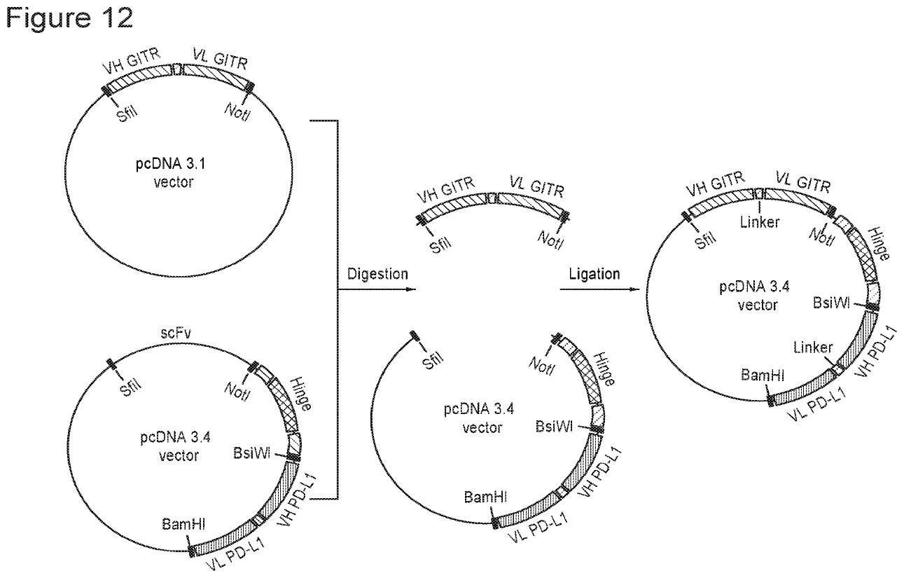

The mammalian expression vector pcDNA3.4 plasmid was the basis for constructs (V_{H}GITR-linker-V_{L}GITR-linker-hinge-linker-V_{H}PD-L1-linker-V_{L}PD-L1). The fundamental structure of pcDNA 3.4 expression vector contained beforehand the V_{H}^{X}-linker-V_{L}^{X}-linker-hinge-linker-V_{H}PD-L1-linker-V_{L}PD-L1 gene fused to an N-terminal 6×-His tag (SEQ ID NO: 1084) (FIG. 12).

Restriction Enzyme Digestion and Ligation

Six αGITR scFv gene sequences were individually cloned into the pcDNA3.4 expression vector. The six V_{H}GITR-linker-V_{L}GITR gene sequences were labeled as V_{H}GITRL1-V_{L}GITRL1, V_{H}GITRL10-V_{L}GITRL10, V_{H}GITRL11-V_{L}GITRL11, V_{H}GITRL14-V_{L}GITRL14, V_{H}GITRL15-V_{L}GITRL15 and V_{H}GITRL17-V_{L}GITRL17. All six αGITR gene sequences are flanked by SfiI and NotI restriction sites and were isolated from the respective donor plasmids by digestion (Table 1). Similarly, the pcDNA3.4 expression vector was also digested with the SfiI and NotI restriction enzymes. The digested vectors and fragments were analyzed on a 1% agarose gel and were purified using a QIAquick Gel Extraction Kit. Cohesive inserts from the SfiI and NotI digestion were ligated into the corresponding vector pcDNA 3.4 at a fivefold molar ratio using the T4 Ligation Kit. Fifty nanograms of the recipient vector were used per ligation reaction. The ligation product resulted in the final configuration of V_{H}GITR-linker-V_{L}GITR-linker-hinge-linker-V_{H}PD-L1-linker-V_{L}PD-L1 (FIG. 12).

Three additional clones were constructed each to generate the control antibodies. The F10 gene was chosen as the ‘control arm’; since V_{H}F10-V_{L}F10 binding domain does not have binding affinity to GITR nor to PD-L1 protein. Thus, this domain was defined as negative control. F10 is a validated antibody directed against influenza HA protein. To keep the same antibody format, its gene sequences only replace either V_{H}GITR1-linker-V_{L}GITR1 or V_{H}PD-L1-linker-V_{L}PD-L1, respectively. The three control plasmids exhibit the following sequencing order:
(1) V_{H}F10-linker-V_{L}F10-linker-hinge-linker-V_{H}PD-L1-linker-V_{L}PD-L1 (F10-αPD-L1)
(2) V_{H}GITR1-linker-V_{L}GITR1-linker-hinge-linker-V_{H}F10-linker-V_{L}F10 (αGITR1-F10)
(3) V_{H}GITR10-linker-V_{L}GITR10-linker-hinge-linker-V_{H}F10-linker-V_{L}F10 (αGITR10-F10)

Construction of Plasmid (1):

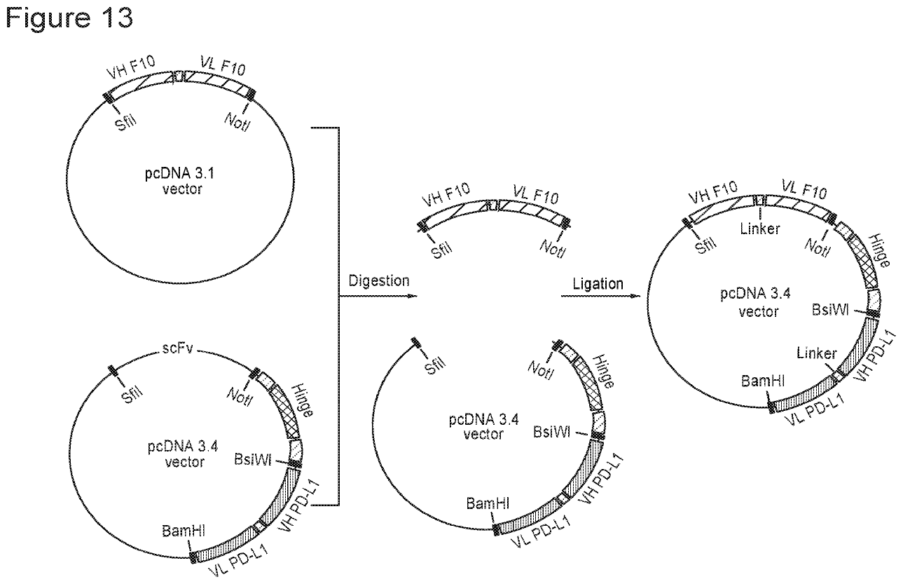

The V_{H}F10-linker-V_{L}F10 gene was isolated from a pcDNA 3.1 vector through the digestion with SfiI and NotI RE. As for the expression vector, the same pcDNA3.4 vector was used (FIG. 13). It contains the SfiI and NotI restriction sites at the desired site of insertion and was therefore digested with the corresponding restriction enzymes. The final plasmid was obtained by ligating both digested products to each other. The ligation was performed using T4 Ligation Kit at 16° C. overnight.

Procedure for the Construction of Plasmids (2) and (3):

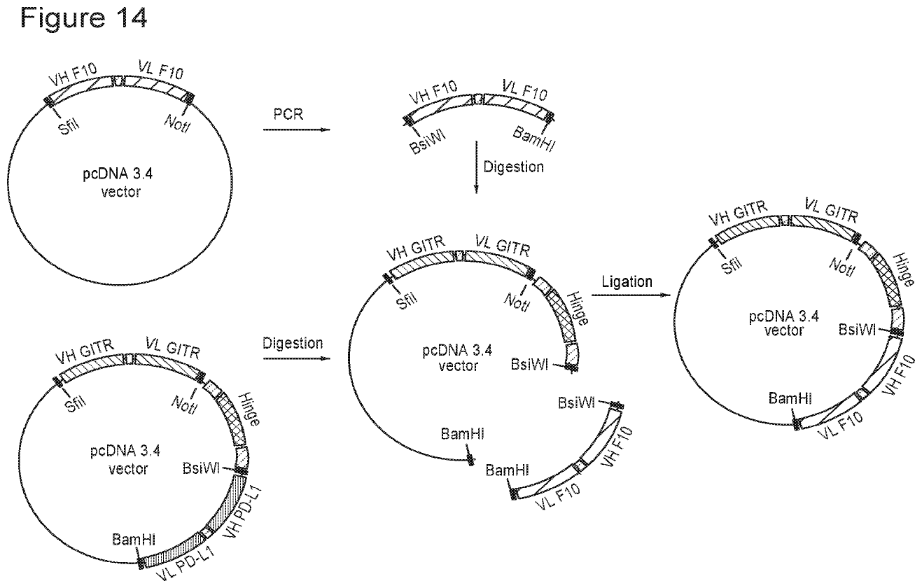

In order to replace the αPD-L1 scFv in the previous construct, a forward and a reverse primer were designed and synthesized to introduce the BsiWI and BamHI restriction site and the 5′ and 3′ of the V_{H}F10-linker-V_{L}F10 fragment, respectively. After PCR amplification, the PCR products containing V_{H}F10-linker-V_{L}F10 and the pcDNA3.4 expression vector were digested with BsiWI and BamHI and used to replace the DNA fragments encoding V_{H}PD-L1-linker-V_{L}PD-L1 within the previously constructed expression plasmids, V_{H}GITR¹-linker-V_{L}GITR¹-linker-hinge-linker-V_{H}PD-L1-linker-V_{L}PD-L1 and V_{H}GITR¹⁰-linker-V_{L}GITR¹⁰-linker-hinge-linker-V_{H}PD-L1-linker-V_{L}PD-L1. The digested expression vector and inserts were gel-purified (1% agarose) using QIAquick gel extraction Kit and subsequently ligated to each other by Quick ligation (5 minutes, at RT). This procedure yielded the plasmids (2) and (3) (FIG. 14).

Primer Design for the Construction of the Control Plasmid Constructs

As described above, two primers were designed for control plasmids (2) and (3) to isolate V_{H}F10-linker-V_{L}F10. The forward primer (5′-3′) was designed to bind on the 3′ prime end of the complementary strand of the DNA; the reverse primers (3′-5′) were designed to bind on the 3′ prime end of the main DNA strand and were reverse complementary. The primers were about 20 bp long with an optimal melting temperature between 62 and 65° C. and not deviating more than ±1° C. The forward primer (containing the BsiWI restriction site (No. 1) and the reverse primer (containing the BamHI restriction site (No. 2)) were synthesized by Genewiz. For the PCR reaction 100 ng DNA template (pcDNA 3.1) was used in the thermal cycling. The PCR product was purified with a QIAquick PCR Purification Kit according to the manufactures protocol and analyzed on a 1% agarose gel.

### Example 2: Cloning of αGITR10-αPD-L1 Tetrameric Bispecific Antibody (tBsAb) Containing a CH2 Domain

Cloning Strategy

The aim of this example was to introduce a CH2 domain from an IgG1 into the previously-constructed plasmid, leading to the basic structure of V_{H}GITR-linker-V_{L}GITR-linker-hinge-CH2-linker-V_{H}PD-L1-linker-V_{L}PD-L1. The addition of CH2 adds an effector function, resulting in a trifunctional tBsAb.

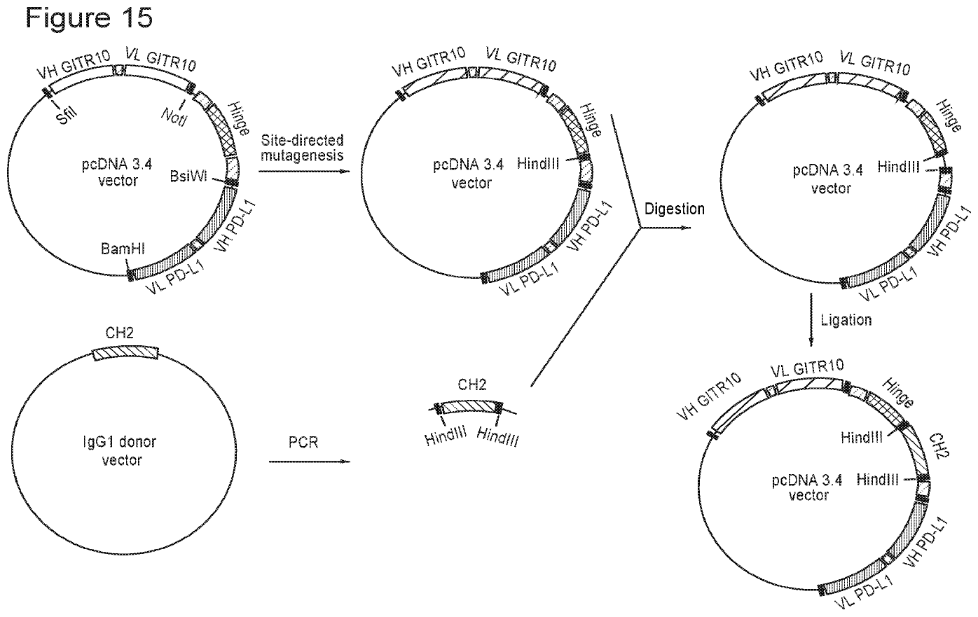

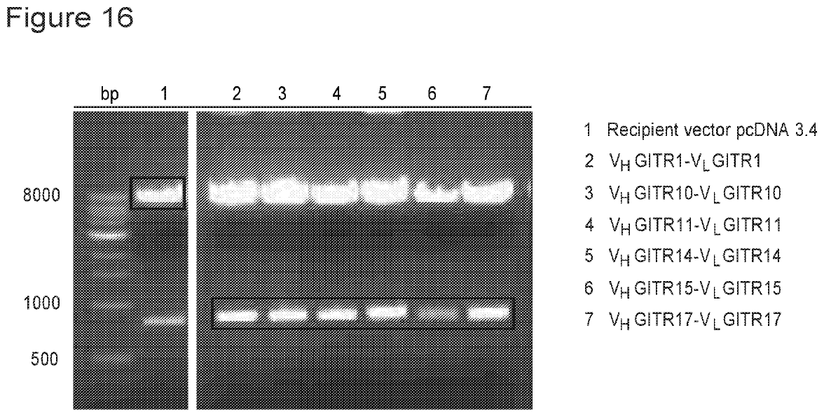

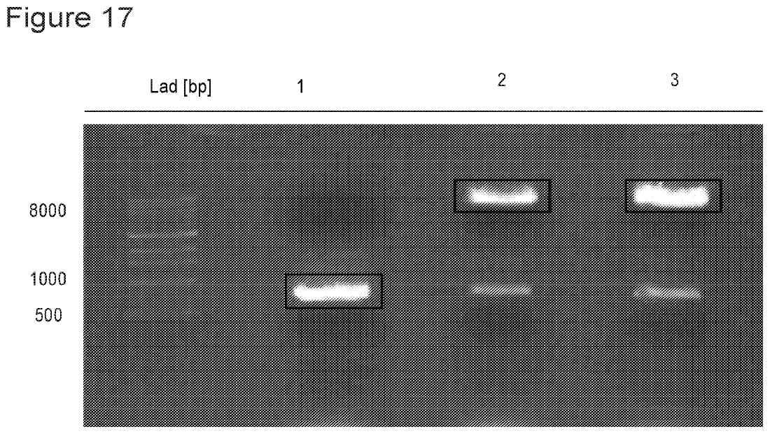

The pcDNA3.4 expression vector containing αGITR10-αPDL1 served as the template used for the construction of the new plasmid. A new restriction site HindIII was introduced by site-directed mutagenesis between the IgG1 hinge region and the linker (GGGGS)₆ (SEQ ID NO: 1909). This newly constructed restriction site served as the cloning site for the IgG1 constant CH2 domain (see FIG. 15). The HindIII restriction site was chosen for several reasons. The HindIII restriction site is unique in the plasmid and its genomic sequence is not similar to its adjacent coding region. Nevertheless, HindIII features some drawbacks such as the relative long length (6 nucleotides) possibly diminishing the mutagenesis efficacy.

The IgG1 plasmid was used as a template to isolate the CH2 domain. The CH2 sequence was amplified by PCR using primers containing the restriction site HindIII. The pcDNA 3.4 expression vector (V_{H}GITR¹⁰-linker-V_{L}GITR¹⁰-linker-hinge-HindIII*-V_{H}PD-L1-linker-V_{L}PD-L1) and the amplified CH2 fragments were digested with the corresponding restriction enzymes. The digested vectors and fragments were gel-purified (1% agarose) using QIAquick gel extraction Kit. Cohesive inserts from the HindIII digest were ligated into the vector (pcDNA 3.4) with twentyfold insert using the Quick Ligation Kit resulting in the construction of a new plasmid V_{H}GITR¹⁰-linker-V_{L}GITR¹⁰-linker-hinge-CH2-linker-V_{H}PD-L1-linker-V_{L}PD-L1.

Site-Directed Mutagenesis

Mutagenesis of the GITR10-PDL1 vector was accomplished with the use of QuikChange Lightning Site-Directed Mutagenesis Kit (Aligent technologies®) following the manufacturer's protocol. Two oligonucleotide primers were synthesized, each complementary to the opposite strand of the vector. Both primers contained HindIII as the desired mutation.

The primers were designed to exhibit the HindIII mutation in the middle of the primer flanked by 7 to 10 bases. The oligonucleotide primers were used for extension by PfuUltra HF DNA Polymerase during temperature cycling. This approach enabled the generation of a mutated plasmid containing staggered nicks. During the next temperature cycle, the product was treated with DpnI to digest the parental DNA template containing methylated and hemimethylated DNA. As a control, the 4.5-kp pWhitescript plasmid was used to test the mutant plasmid. The pWhitescript plasmid codes a stop codon (TAA) at the position where a glutamine codon would appear in the β-galactosidase gene of the pBluescript II, usually obliterating the blue color of the colonies on LB-ampicillin agar plate containing IPTG and Xgal. However, the oligonucleotide control primers create a point mutation on the pWhitescript 4.5-kb control plasmid that reverts the T residue of the stop codon to C, thereby producing the phenotype of blue color on media containing IPTG and X-gal. After the cycling, 2 μL of the DpnI restriction enzyme was added (37° C., 5 min) to digest the parental dsDNA. The mutagenesis plasmid was then transformed into XL10Gold® Ultracompetent cells and spread on LB-ampicillin agar plates containing 80 μg/ml X-gal and 20 mM IPTG (37° C.; >16 hours). On the following day, 16 clones were picked from the LB-ampicillin plates, purified using QIAprep spin Miniprep Kit and digested with HindIII and NotI restriction enzymes to identify successfully-mutated clones. Positive clone No. 10 (GITR10-PDL1 with HindIII) was subjected to another digestion to compare it with the original plasmid GITR10-PDL1 (no HindIII). Each sample was individually digested with HindIII or BamHI and simultaneously with HindIII and BamHI-HF together, resulting in a total of six digestions (see Table 1 below).

TABLE 1
Parameters and volumes for the total six restriction
enzyme digestions

| | GITR10-PDL1 with | | | | | |
|---|---|---|---|---|---|---|
| Digestion | HindIII restriction site | | | GITR10-PDL1 | | |
| Parameters | 1 | 2 | 3 | 1 | 2 | 3 |
| Plasmid | 3.1 | 3.1 | 3.1 | 3.1 | 3.1 | 3.1 |
| (2.0 μg) [μL] | | | | | | |
| 10x Cutsmart | 3.0 | 3.0 | 3.0 | 3.0 | 3.0 | 3.0 |
| (NEB ®) [μL] | | | | | | |
| HindIII-HF | 0.5 | — | 0.5 | 0.5 | — | 0.5 |
| (20,000 U/mL, | | | | | | |
| NEB ®) [μL] | | | | | | |
| BamHI-HF | — | 0.5 | 0.5 | — | 0.5 | 0.5 |
| 20,000 | | | | | | |
| U/mL [μL] | | | | | | |
| ddH2O | ad. 30 | ad. 30 | ad. 30 | ad. 30 | ad. 30 | ad. 30 |
| (Mili-Q ®) | | | | | | |
| [μL] | | | | | | |

The six samples were incubated for 2 hours at 37° C. and analyzed in a 1% agarose gel.

The bacteria containing the positively-mutated clone No. 10 were amplified overnight at 37° C. in 120 mL YT medium followed by plasmid DNA purification, using the QIAGEN Plasmid Maxi Kit. The correct construct, containing HindIII restriction site, was confirmed by sequencing (Genewiz®, using pre-designed primers. A glycerol stock was prepared and stored at −80° C. The recipient plasmid containing HindIII domain and the CH2 fragment were digested with HindIII and subsequently ligated to each other. Ligation products were transformed by heat-pulse into XL10-Gold® Ultracompetent cells according to the protocol described herein. The correct plasmid was verified by sequencing (Genewiz®).

Transformation

Ligation products were transformed by heat-pulse into XL10-Gold® Ultracompetent cells. These cells were gently thawed on ice. For each transformation, 45 μL of the cells were mixed with 2 μL of B-Mercaptoethanol and 1.5 μL of the interested DNA. The transformation reaction was incubated for 30 minutes followed by heat-pulse in a 42° C. water bath for 40 seconds. 0.5 mL of S.O.C Medium (Life Technologies®) was added to each tube and incubated for one hour at 37° C. The transformation reaction was grown overnight on LB-ampicillin plates at 37° C.

Several colonies per ligation sample were picked individually and grown in 1.5 mL 2-YT medium for 8 hours. Plasmids of the picked clones were purified using the QIAprep Spin Miniprep Kit as specified by the manufacturer. Correct plasmids were verified by sequencing (Genewiz®). Bacteria of positive clones were grown overnight in 120 mL YT medium (37° C., 240 rpm) and plasmid DNA was purified using the QIAGEN Plasmid Maxi Kit (as per manufacturer's protocol). Glycerol stocks were prepared by adding 400 μL glycerol and 600 μL of the culture into cryotube vials and then stored at −80° C.

Cell Culture & Transfection

For protein expression 293F human cell lines 293F were obtained from Life Technologies® and 293 T adherent cell line from the ATCC cell bank. For cell-based ELISA assays, CF2-GITR cell lines were generated in the Marasco Laboratory to express GITR on cell surface.

293F Cells in Suspension for Protein Expression

Suspension cultures of 293F cells (derived from human embryonic kidney cells; HEK cells) were maintained in Erlenmeyer flasks (Corning®) and in 293 freestyle medium (Life Technologies®) at 37° C. and with 5% CO₂. Cells were passaged during log growth phase and were diluted into an optimal density (200,000 cells/mL) with fresh medium for continued growth.

293T and the CF2-GITR Adherent Cells

Adherent 293T or CF2-GITR cells were maintained in 75 cm2 flasks (Cellstar) and DMEM medium (Life Technologies®) supplemented with 10% FBS (fetal bovine serum) (Life Technologies®) and 1% SP (Sodium Pyruvate) (Life Technologies®) at 37° C. in 5% CO₂. Cells were passaged at 80-100% confluency and were diluted to an optimal seeding density (2×10⁶ cells) with fresh medium for continued growth.

Transfection

For the production of the tetrameric bispecific antibody (tBsAb) (αGITR1-αPD-L1, αGITR10-αPD-L1, αGITR11-αPD-L1, αGITR14-αPD-L1, αGITR15-αPD-L1, αGITR17-αPDL1 and αGITR10-αPD-L1 (with CH2)) and the control antibodies (αGITR1-F10, αGITR10-F10, F10-αPD-L1, αGITR IgG), 293 F or 293T cells were transfected with the respective plasmids.

Polyethlenimine (PEI)-Mediated Transient Transfection in 293F HEK Cells

One day before transfection, cells were passed into a final concentration of 6×10⁵ cells/mL in a total volume of 300 mL. On the day of transfection, the cell density was between 1.0×10⁶ and 1.4×10⁶ cells/mL. The respective plasmids were prepared for transfection. The overall charge of the transfection complexes is determined by the ratio of transfection reagent to the DNA. The negative charge contributed by the phosphates in the DNA backbone is offset by the positive charge of the transfection reagent. This allowed good complex formation and for neutralization of the electrostatic repulsion imparted on the DNA by the negatively-charged cell membrane. A 1:1 ratio of Plasmid:PEI allowed full binding of polymer to DNA and full condensation occurred to protect the cargo; however, the excess of PEI is critical for overcoming the inhibitory effects of the anionic cell surface. For every million cells, 1 μg plasmid and 3 μg PEI were used for transfection and each was diluted into 15 mL Opti-MEM (Reduced Serum Medium) (Life Technologies®) separately. Diluted PEI was added to the plasmid and incubated at RT for 20 minutes. The efficiency of neutralization increases with time of exposure to the PEI-DNA complex; however excessively long exposure to lipid reagents can be toxic. The PEI/Plasmid complex was poured into the 293F suspension cells (1×10⁶ cells/mL; 300 mL per flask) and was incubated at 37° C. at 140 rpm for 6 days.

Polyethlenimine (PEI) Mediated Transient Transfection in 293T HEK Cells

Transfection of 293T HEK with use of PEI cells follows the same protocol as described above for the 293F suspension HEK cells with a couple of small changes. Transfection was done on 293T cells growing at 80% confluency in tissue culture dishes (200 mm) diluted DMEM medium supplemented with 10% FBS. For 40 μg of DNA, 200 μg of PEI was used (a 1:5 ratio) and each was diluted separately in 1 mL Opti-MEM (Reduced Serum Medium) (Life Technologies®) separately. The diluted PEI was added to the plasmid and stored at RT for 20 minutes. The DNA/PEI complex was added gently, drop wise, into the dish to prevent cell detachment and death. The cells were then incubated at 37° C. for 2 days.

### Example 3: Protein Purification

Ni-NTA Purification of Bispecific Antibody Antibodies

Suspension of 293 HEK cells were harvested and centrifuged at 5000 rpm, 4° C. for 35 minutes. To purify the bi-specific antibodies via their N-terminal 6× His-tags (SEQ ID NO: 1084), the filtered supernatant (0.22 μm PEV, Costar®) was incubated for 2 hours (240 rpm, RT) with 1 mL of Ni-NTA agarose (Qiagen). The supernatant was passed twice over a 15 ml Ni-NTA Sepharose gravity flow column. After washing, the column containing the beads were washed with four column volumes of Ni-NTA washing buffer (0.02M Imidazole, 0.3M NaCl, 1M Tris HCl (pH=7.0)) the protein was slowly eluted with 13 mL of Ni-NTA elution buffer (0.5M Imidazole, 0.3 NaCl, 0.02 Tris HCl (pH=7.0)). The buffer of the eluted protein was exchanged by PBS buffer using centrifugal filter units 100,000 MW (Amicon®). The yield of the tBsAbs and was measured with the NanoDrop ND-1000.

Protein A Purification of αGITR IgG Antibody

The αGITR IgG antibodies were harvested from the suspension of 293 HEK cells and centrifuged at 5000 rpm, 4° C. for 35 minutes. To purify the αGITR IgG antibodies via the Fc domain, the filtered supernatant was incubated (RT, shaking) with 1 mL of Protein A (GE Lifesciences) for 2 hours then passed twice over a 15 ml gravity flow column (Biorad) followed by 10 mL PBS for washing. The αGITR IgG was eluted with 2 ml TEA (100 nM) and 200 μL of Tris-HCl (1M, (pH=7)) was added to the elution in order to neutralize TEA. An additional 2 mL PBS was added onto column, and was collected into the tube with the eluted protein.

### Example 4: Protein Characterization

SDS-PAGE Analysis

SDS-PAGE analysis was used to verify the purity of proteins, according to the NuPAGE NuPAGE® technical guide (Invitrogen). NuPAGE Bis-Tris Gels (4-12%) (Novex) were used in MES SDS running buffer, with a total protein amount between 3 μg and 5 μg. The protein samples were mixed with 4× LDS sample buffer (Novex), containing dodecylsulfate, to denature the protein. Under reducing conditions the protein samples were additionally boiled at 100° C. for 10 minutes. The samples were then loaded onto Novex Bis-Tris Gels in MES SDS running buffer. The gel was run in a Xcell SureLock Mini-Cell at 200V for 35 minutes and then processed with Coomassie G-250 staining with simplyBlue™ Safe Stain (Novex).

Direct ELISA of αGITR-αPD-L1 on Passively Adsorbed Soluble PD-L1 Antigen

A Maxisorb 96 well plate (Costar®) was coated with 100 μL of 5 μg/mL PD-L1 rabbit Fc antigen and CCR4 protein (negative control) in PBS overnight at RT. On the following day, the plate was washed 3 times with PBS and blocked for 2 hours at RT with 200 μL blocking solution (2% BSA in PBS). The plate was washed 3 times with PBS. The primary antibodies αGITR1-αPD-L1, αGITR10-αPD-L1, αGITR11-αPD-L1, αGITR14-αPD-L1, αGITR15-αPD-L1, αGITR17-αPD-L1, F10-αPD-L1 BsAB and commercial anti-mouse PD-L1 mAb (Biolegend) prepared in 1× PBS with variable concentrations and added to the wells (100 μL) for 2 hours incubation at room temperature. The highest concentration of antibody tested was at 1 μg/mL and then serially diluted in a ten-fold manner until the 1×10⁻⁵ μg/mL dilution. Each sample was run in triplicate at every concentration. Several controls were set-up and are listed in the table below (Table 2). The 96 well plate (Costar) was washed three times with 1× PBS buffer. A solution of the secondary antibody (6× His-HRP (Thermoscientific) (“6× His” disclosed as SEQ ID NO: 1084) and Goat anti-mouse IgG Fc, HRP conjugate (Thermoscientific) were diluted (1:2000 and 1:5000) in 1× PBS. The secondary antibodies (100 μL) were added to each well and incubated for 2 hours at room temperature. Finally, each well was washed four times with PBS. The 96 well plate was developed with 100 μL TBM substrate solution (Thermoscientific); after development 100 μL phosphoric acid stop solution (Thermoscientific) was added. The endpoint OD data was recorded at 450 nm with Bio-Rad Benchmark Plus and analyzed with the Microplate Manager 5.2.1 software.

TABLE 2
Experimental Overview on tested samples and controls
for direct ELISA of αGITR-αPD-L1 on passively adsorbed
PD-1 antigen (“6xHis” disclosed as SEQ ID NO: 1084)

| | Test- | Primary | Secondary | | Expected |
|---|---|---|---|---|---|
| Antigen | Type | AB | AB | Purpose | signal |
| PD-L1 | Sample | αGITR- | Anti | Sample | Positive |
| rabbit | | αPD-L1 | 6xHis-HRP | testing | |
| Fc | | tetrameric | (Thermo- | | |
| fusion | | bispecific | scientific) | | |
| protein | | antibodies | | | |
| | | Commercial | Goat | Standard and | Positive |
| | | mouse anti | anti-mouse | verification | |
| | | human | IgG Fc, HRP | that PD-1 | |
| | | PD-L1 mAb | conjugate | rabbit Fc | |
| | | (Biolegend) | (Thermo- | can be | |
| | | | scientific) | labeled | |
| | Control | No | Anti | Measure | Negative |
| | | | 6xHis-HRP | unspecific | |
| | | | (Thermo- | binding | |
| | | | scientific) | | |
| | | No | Goat | Measure | Negative |
| | | | anti-mouse | unspecific | |
| | | | IgG Fc, HRP | binding | |
| | | | conjugate | | |
| | | | (Thermo- | | |
| | | | scientific) | | |
| CCR4- | Control | Commercial | Anti | Verification | Negative |
| human | | anti mouse | 6xHis-HRP | of specific | |
| Fc | | PD-L1 mAb | (Thermo- | binding of | |
| | | (Biolegend) | scientific) | samples | |
| | | No | Anti | Measure | Negative |
| | | | 6xHis-HRP | unspecific | |
| | | | (Thermo- | binding | |
| | | | scientific) | | |
| | | No | Goat | Measure | Negative |
| | | | anti-mouse | unspecific | |
| | | | IgG Fc, HRP | binding | |
| | | | conjugate | | |

Cell-Based ELISA of αGITR-αPD-L1 BsAbs on GITR⁻ CF2

For the cell-based ELISA, the αGITR1-αPD-L1, αGITR10-αPD-L1 and αGITR10-αPD-L1 with CH2 antibodies were tested for retained binding capability on GITR+CF2 cells. In total, four ELISA experiments were set up.

The first cell-based ELISA the αGITR1-F10 and the αGITR10-F10 tetrameric bispecific antibodies (tBsAbs) were analyzed. For GITR+ CF2 and GITR− CF2 cell (negative control) seeding, 1,000 cells per well were added in 200 μL of 1% DNEM medium and were incubated overnight to allow attachment. On the following day, the cells were fixed with 100 μL of Acetone-Methanol solution (1:1 ratio) and incubated for 20 minutes at RT. The Acetone-Methanol solution was aspirated from the plate and the cells were washed three times with 1× PBS. The general assay procedure and development was performed according to the protocol for ELISA mentioned in chapter 2.6.2. The primary antibodies αGITR1-αPD-L1 and αGITR10-αPD-L1 were tested in variable concentrations. The tBsAbs were serially diluted by one third in 1× incubation buffer; 3.33 mg/mL being the highest concentration and 0.0411 mg/mL the lowest. Several controls were set-up and are listed on the table below (Table 3).

TABLE 3
Experimental Overview on tested samples and controls for
cell based ELISA of αGITR1-αPD-L1 and αGITR10-
αPD-L1 on GITR+CF2 cells (“6xHis” disclosed as SEQ ID NO: 1084)

| | Test- | Primary | Secondary | | Expected |
|---|---|---|---|---|---|
| Antigen | Type | AB | AB | Purpose | signal |
| GIRT⁺CF2 | Sample | αGITR1- | Anti | Sample | Positive |
| | | αPD-L1 | 6xHis-HRP | test | |
| | | | (Thermo- | | |
| | | | scientific) | | |
| | | αGITR10- | Anti | Sample | Positive |
| | | αPD-L1 | 6xHis-HRP | test | |
| | | | (Thermo- | | |
| | | | scientific) | | |
| | | F10- | Anti | Negative | Negative |
| | | αPDL1 | 6xHis-HRP | control | |
| | | | (Thermo- | | |
| | | | scientific) | | |
| | Control | No | Anti | Unspecific | Negative |
| | | | 6xHis-HRP | binding | |
| | | | (Thermo- | | |
| | | | scientific) | | |
| GITR⁻ CF2 | Sample | αGITR1- | Anti | Sample | Negative |
| | | αPD-L1 | 6xHis-HRP | test | |
| | | | (Thermo- | | |
| | | | scientific) | | |
| | | αGITR10- | Anti | Sample | Negative |
| | | αPD-L1 | 6xHis-HRP | test | |
| | | | (Thermo- | | |
| | | | scientific) | | |
| | | F10- | Anti | Sample | Negative |
| | | αPDL1 | 6xHis-HRP | | |
| | | | (Thermo- | | |
| | | | scientific) | | |
| | Control | No | Anti | Unspecific | Negative |
| | | | 6xHis-HRP | binding | |
| | | | (Thermo- | | |
| | | | scientific) | | |

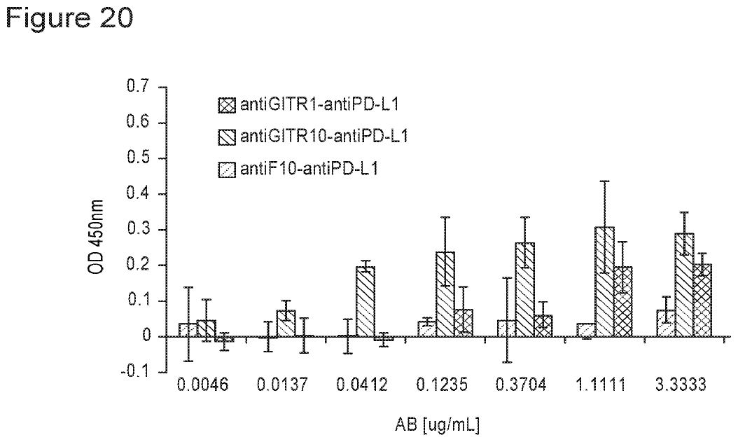

After evaluating the results of the cell-based ELISA (FIG. 20), the experiment for a second cell-based ELISA was repeated using the same procedure to that described above, except that cells were fixed with 8% paraformaldehyde.

A third cell based ELISA was performed to compare αGITR10-αPD-L1 tBsAb to the commercial human αGITR mAb. For GITR⁺ CF2 and GITR⁻ CF2 cell (negative control) seeding, 10,000 cells per well were added in 200 μL of 1% DMEM medium and were incubated overnight to allow attachment. On the following day, the cells were fixed with 100 μL of 8% paraformaldehyde and incubated for 20 minutes at RT. The paraformaldehyde solution was aspirated from the plate and the cells were washed three times with 1× PBS. The general assay procedure and development was performed according to the protocol for ELISA mentioned herein. The primary antibodies αGITR10-αPD-L1 and αGITR mAb were tested in variable concentrations. The antibodies were serially diluted (1:2) in 1× incubation buffer; 5 mg/mL being the highest concentration and 0.078 mg/mL the lowest. Several controls were set-up and are listed on the table below (Table 4).

TABLE 4
Experimental Overview on tested samples and controls
for cell based ELISA of αGITR10-αPD-L1 and αGITR
mAb on GITR+CF2 cells

| | Test- | Primary | Secondary | | Expected |
|---|---|---|---|---|---|
| Antigen | Type | AB | AB | Purpose | signal |
| GITR⁺CF2 | Sample | αGITR10- | His-HRP | Sample | Positive |
| | | αPD-L1 | | test | |
| | | Commercial | Goat anti- | Standard | Positive |
| | | GITR mAb | mouse | | |
| | | (Purified | IgG | | |
| | | anti-hu | Fc, HRP | | |
| | | CD357)(Bio- | conjugate | | |
| | | legend) | (Thermo- | | |
| | | | scientific) | | |
| | | F10- | His-HRP | Control | Negative |
| | | αPDL1 | | | |
| | Control | No | His-HRP | Measure | Negative |
| | | | | unspecific | |
| | | | | binding | |
| | | No | Goat anti- | Measure | Negative |
| | | | mouse | unspecific | |
| | | | IgG | binding | |
| | | | Fc, HRP | | |
| | | | conjugate | | |
| | | | (Thermo- | | |
| | | | scientific) | | |
| GITR⁻ CF2 | Sample | αGITR10- | His-HRP | Sample | Negative |
| | | αPD-L1 | | test | |
| | | Commercial | Goat anti- | Standard | Negative |
| | | GITR mAb | mouse | | |
| | | (Purified | IgG | | |
| | | anti-hu | Fc, HRP | | |
| | | CD357)(Bio- | conjugate | | |
| | | legend) | (Thermo- | | |
| | | | scientific) | | |
| | | F10- | His-HRP | Control | Negative |
| | | αPDL1 | | | |
| | Control | No | His-HRP | Measure | Negative |
| | | | | unspecific | |
| | | | | binding | |
| | | No | Goat anti- | Measure | Negative |
| | | | mouse | unspecific | |
| | | | IgG | binding | |
| | | | Fc, HRP | | |
| | | | conjugate | | |
| | | | (Thermo- | | |
| | | | scientific) | | |

The fourth ELISA was performed to compare αGITR10-αPD-L1 with CH2 tBsAb to the commercial αGITR mAb. The assay procedure was identical to the third ELISA (mentioned above).

Flow Cytometry Analysis for αGITR1-αPD-L1 & αGITR10-αPD-L1

The biological activity of αGITR on GITR+CF2 cells was analyzed by means of fluorescence-activated cell sorting FACS analysis. The cells GITR+CF2 cells and GITR−CF2 were grown in a 75 cm² flask (Cellstar) until they reached roughly 80% confluence. They were detached by adding 1:10 diluted Trypsin with 0.25% Trypsin-EDTA (Life Technologies) in PBS and resuspended and then added to 96-well round bottom plate in FACS buffer (PBS, 1% FBS, 2 mM EDTA). In the following step the αGITR1-αPD-L1 and αGITR10-αPD-L1 were added at variable concentrations for 1 hour at 4° C. The highest concentration of antibody tested was at 100 μg/mL and then serially diluted in a two-fold manner until the 0.05 μg/mL dilution. The primary antibodies were detected with His Tag Alexa Fluor 488-conjugated (Biotechne). The secondary antibody was diluted in PBS (Life Technologies) and added to each well for 30 minutes. The cells were then washed three times with PBS buffer and resuspended in FACS buffer. In total 10,000 events were analyzed with FACSCalibur. Results were analyzed by FlowJo 10.1 software. Several controls were performed and are listed in the table below. (Table 5)

TABLE 5
Experimental Overview on control samples for
FACS analysis of αGITR1-αPD-L1α and
GITR10-αPD-L1 on GITR+CF2 cells and GITR−CF2 cells.

| | Test- | Primary | Secondary | | Expected |
|---|---|---|---|---|---|
| Antigen | Type | AB | AB | Purpose | signal |
| GITR⁺CF2 | Control | none | Alexa | Unspecific | negative |
| | | | 488; | binding of | |
| | | | anti- | secondary | |
| | | | His | AB | |
| | | | (APC) | | |
| | | F10- | Alexa | Exclude | negative |
| | | αPD-L1 | 488; | the PD-L1 | |
| | | | anti- | binding to | |
| | | | His | GITR+ | |
| | | | (APC) | CF2 | |
| | | αGITR1 | Anti- | Verify that | positive |
| | | mAb | human | αGITR | |
| | | | IgG Fc | arm can | |
| | | | (FITC) | recognize | |
| | | | | the GITR | |
| | | | | binding | |
| | | | | site | |
| | | none | Anti- | Unspecific | negative |
| | | | human | binding of | |
| | | | IgG Fc | secondary | |
| | | | (FITC) | | |
| | | Commercial | Anti- | Verify that | positive |
| | | mouse | mouse | CF2 cells | |
| | | αGITR | IgG Fc | are | |
| | | IgG | (FITC) | expressing | |
| | | | | GITR | |
| | | none | Anti- | Unspecific | negative |
| | | | mouse | binding of | |
| | | | IgG Fc | secondary | |
| | | | (FITC) | | |
| GITR⁻ CF2 | Control | none | Alexa | Unspecific | negative |
| | | | 488; | binding of | |
| | | | anti-His | secondary | |
| | | | (APC) | | |
| | | F10- | Alexa | Exclude | negative |
| | | αPD-L1 | 488; | the PD-L1 | |
| | | | anti- | binding to | |
| | | | His | GITR+ | |
| | | | (APC) | CF2 | |
| | | αGITR1 | Anti- | Verify that | negative |
| | | IgG | human | αGITR | |
| | | | IgG Fc | arm binds | |
| | | | (FITC) | specif- | |
| | | | | ically | |
| | | | | to GITR+ | |
| | | | | CF2 cells | |

### Example 5: Functional Studies

ADCC Assay of αGITR-αPDL1 with CH2 on GITR+CF2 Cells

The antibody-dependent cell-mediated cytotoxicity of αGITR-αPD-L1 with CH2 on GITR+CF2 cells was analyzed using ADCC Reporter Bioassay Complete Kit (WIL2-S) (Promega) and implemented according to the manufacturers protocol. The aim was to test the αGITR10-αPD-L1 with CH2 for ADCC. The assay was performed using ADCC reporter cells (WIL2-S) that have Fcy receptors and the response element-driven luciferase gene.

The cells GITR⁺CF2 cells and GITR⁻CF2 were grown in a 75 cm² flask (Cellstar) until they reached roughly 80% confluence. They were detached by adding 1:10 diluted 0.25% Trypsin-EDTA (Life Technologies) in PBS and tested for viability. The GITR⁺CF2 cells were used as target cells and plated in 96-well cell flat bottom microplate (PerkinElmer) at a density of 2×10⁴ cells per well diluted in RPMI 1640 medium (Life Technologies®; serum free). The αGITR10-αPD-L1 (with CH2) and the controls (αGITR10-IgG (positive control) and GITR10-PD-L1 and F10-PDL1 (negative control) were serially diluted in ADCC assay medium. The four antibodies were added in a concentration-dependent manner, starting at 20 mg/mL (highest concentration), followed by 2 mg/mL, 0.2 mg/mL and 0.02 mg/mL, respectively (1:10 serial dilution) and incubated for 5 minutes at RT. Following incubation, the effector cells WIL2-S were suspended in ADCC assay medium and added to the target cell/antibody mixture at 10×10⁶ cells per well. The ratio of effector cells to target cells was set up as 5:1 (E/T). After approximately a 6 hour incubation at 37° C. (5% CO₂), an equal volume of the Bio-Gio Luciferase assay reagent (Promega) was added to the wells and incubated (RT, 10 min). The luminescence of the cells was measured using Polarstar Omega. Assays were performed in triplicate. All data were plotted using Excel.

CDC Assay of αGITR-α-PDL1 with CH2 on GITR⁺CF2 Cells

For the testing of complement-dependent cytotoxicity (CDC) of the αGITR10-α-PDL1 with CH2 tBsAb, baby rabbit complement (Cedarlane Laboratories) was used in the CellTox™ Green Cytotoxicity assay (Promega) using CellTox Green Dye (Promega) that binds DNA in comprised cells. The fluorescent signal produced by the dye binding to the dead-cell DNA is proportional to cytotoxicity. The assay was performed according the manufacturers protocol. The experimental procedure and set-up for the testing of complement-dependent cytotoxicity was similar to the CDC test mentioned above, with exception of the assay development and analysis which was performed with the CellTox™ Green Cytotoxicity assay (Promega). The antibodies tested for complement-dependent cytotoxicity were the αGITR10-αPDL1 and the αGITR10-α-PDL1 with CH2 tetrameric bispecific antibodies (tBsAbs). The αGITR mAb was used for positive control and the F10-αPD-L1 was used as negative control.

After approximately a 4 hour incubation at 37° C. (5% CO₂) an equal volume of CellTox Green Dye assay reagent (Promega) was added to the wells and incubated (RT, 10 min). The fluorescence was measured using Polarstar Omega. Assays were performed in triplicate. All data were plotted using Excel.

### Example 6: Isolation and Characterization of the αGITR-αPD-L1 BsAbs

Generation of Expression Vector

In total six vectors (αGITR-αPD-L1) were constructed to produce the desired tBsAbs and additional three vectors for the production of control Abs (αGITR1-F10, αGITR10-F10 and F10-αPD-L1). The expression vector was generated according to the cloning strategy described above.

The recipient expression vector pcDNA 3.4 and all donor vectors (six V_{H}GITR-linker-V_{L}GITR and inserts and one V_{H}F10-V_{L}F10 insert) were digested with SfiI and NotI restriction enzymes and the fragments were separated on a 1% agarose gel, stained with ethidium-bromide. The SfiI and NotI digestion patterns of the seven digestions were in agreement with the theoretically calculated values. The digested recipient vector pcDNA 3.4 vector comprises 7500 bp and can be detected at the correct level (lane1; 8000 bp) of the ladder. The smaller fragment in lane 1 displayed between 500 and a 1000 bp and corresponds to V_{H}^{X}-linker-V_{L}^{X} of a previously-used scFv. The GITR inserts (lanes 2-6) and the F10 insert (lane 7) were clustered between 500 and 1000 bp. The larger bands seen at the level of 8000 bp (lanes 2-7) represent the corresponding descendent vectors.

Two additional control plasmids (2) and (3) were constructed. The recipient expression vectors pcDNA3.4 encoding the αGITR1-αPDL1 and αGITR10-αPDL1 scFvs were digested with BsiWI and BamHI Res to replace the V_{H}PD-L1-linker-V_{L}PD-L1 fragment with V_{H}F10-linker-V_{L}F10 fragment. To isolate the V_{H}F10-linker-V_{L}F10 fragment from the pcDNA3.1 donor vector, a forward and a reverse primer (No.1 and No.2) were designed, containing the BsiWI and BamHI restriction site. After isolating the cDNA using PCR, it was digested with BsiWI and BamHI RE. The gel analyses of all 3 digestions were consistent with the theoretical number. As anticipated the PCR of F10 fragment only shows one band at the correct position relative to the ladder. The two digested recipient vectors (containing V_{H}GITR1-V_{L}GITR1 or V_{H}GITR10-V_{L}GITR10, respectively) are approximately 8000 bp in size and match the theoretical size of the vector (7500 bp).

All digested fragments were extracted and purified form the agarose gel and the respective ligation reactions were performed. The yielded plasmids were successfully constructed and confirmed by sequencing (Genewiz).

Expression of GITR-PDL1 bispecific Antibodies and αGITR-IgG The αGITR-αPD-L1 proteins were expressed in 293F HEK cells and isolated via Ni-NTA purification. The αGITR IgG protein was expressed in HEK 293F cells and isolated via Protein A purification. The yields measured by NanoDrop spectrophotometer are listed in table 6.

TABLE 6
Antibody yield of 293F HEK expression

| | Yield per | | per |
|---|---|---|---|
| | 300 mL | | 300 mL |
| | cell culture | | cell culture |
| Antibody | [mg] | Antibody | [mg] |
| αGITR1-αPD-L1 | 3.3 | αGITR17-αPD-L1 | 2.5 |
| αGITR10-αPD-L1 | 5.5 | F10-αPD-L1 | 1.1 |
| αGITR11-αPD-L1 | 2.8 | αGITR1-F10 | 1.4 |
| αGITR14-αPD-L1 | 1.7 | αGITR10-F10 | 1.5 |
| αGITR15-αPD-L1 | 2.7 | αGITR IgG | 2.9 |

SDS-PAGE Analysis

The purity of the tBsAbs αGITR1-αPDL1, αGITR10-αPDL1, αGITR11-αPDL1, αGITR14-αPDL1, αGITR15-αPDL1, αGITR17-αPDL1 and F10-αPD-L1 were analyzed by SDS-PAGE. Between 3 μg-5 μg of protein sample was loaded on the gel, separated electrophoretically and stained with Coomassie blue.

Notable, under non-reducing conditions, there are two bands that particularly draw attention. The upper band lies within the 115 kDa and 140 kDa range. The quantitative predominance of this band in each of the protein profiles and the proximity of its apparent molecular size to that of the αGITR-αPD-L1 tetrameric bispecific antibodies (tBsAbs) (130 kDa) indicate successful antibody production. The lower band lies between 70 and 80 kDa and thus, may account for a substantial amount of monomeric tandem scFv (65 kDa). Apart from this, some weaker bands above 140 kDa are observable, suggesting the formation of aggregates.

Under reducing conditions, only one band can be seen between 70 and 80 kDa, which suggests the disulfide bond reduction of tBsAbs into tandem scFv (65 kDa). Deviating molecular weights between apparent and theoretical calculated values can stem from post-translational modifications (such as glycosylation and phosphorylation) as well as protein conformation as it is running through the SDS PAGE. Differences amounts loaded onto the gel can account for differences in the intensity of the bands between the αGITR-αPD-L1 tBsAbs.

Further, the purity of the αGITR-IgG was also analyzed by SDS-PAGE. Under non-reducing conditions, the analysis exhibited one band with an apparent molecular weight of 140 kDa and is approximate equal to the theoretical calculated molecular weight of an αGITR IgG (150 kDa). The reduced SDS analysis revealed two bands, which proposes the successful disulfide bond reduction of the αGITR IgG resulting in heavy chain (50 kDa) and light chain (25 kDa).

Direct ELISA of αGITR-αPD-L1 BsAbs on Passively Adsorbed PD-L1 Antigen

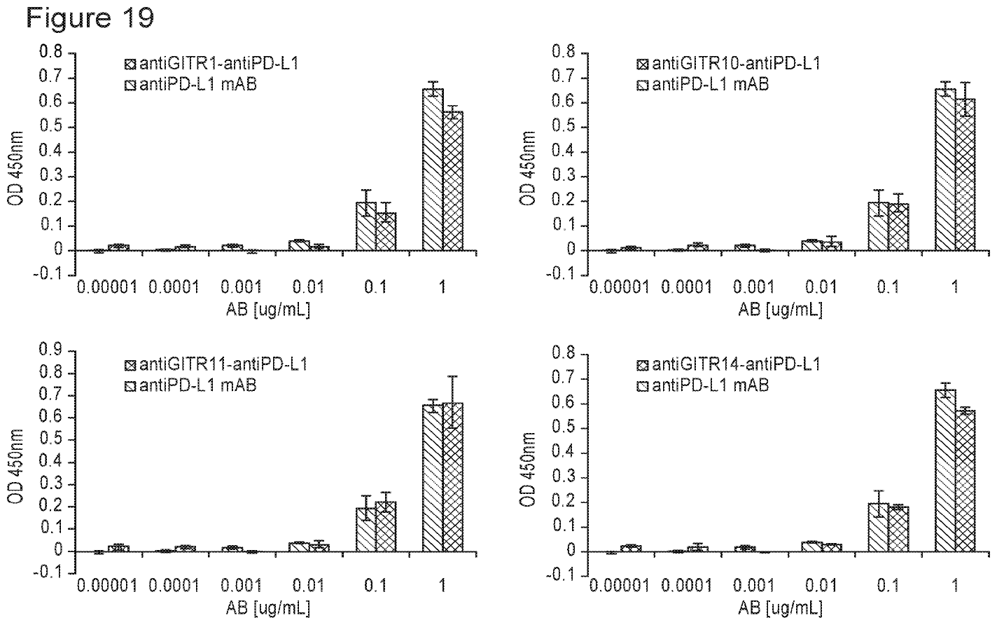

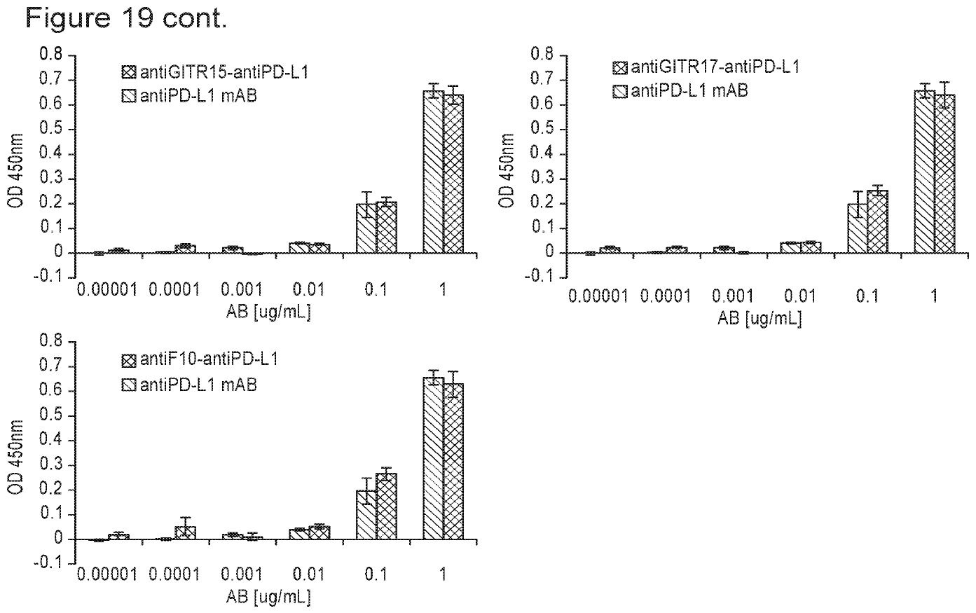

Direct ELISA of the αGITR-αPD-L1 BsAbs was performed to characterize their reactivity to the PD-L1 antigen. As shown in FIG. 19, reactivity to PD-L1 antigen could be observed in all αGITR-αPD-L1 tBsAbs, while no unspecific stickiness to CCR4 was seen (not shown). The readout signal was very similar for all αGITR-αPD-L1 tBsAbs at all concentrations. Highest ELISA signals were measured at highest concentrations. In addition the absorbance value of αGITR-αPD-L1 tBsAbs binding was comparable to that for the commercial αPD-L1 mAb and the intensity signal decreased with lower concentrations. The ELISA does not show saturation at the higher concentration and has very weak signal at concentrations below 0.01 mg/mL.

Cell-Based ELISA of αGITR-αPD-L1 tBsAbs on GITR+ CF2

In previous studies of αGITR IgGs, αGITR1 IgG and αGITR10 IgG were proven to have the best characteristics, which is why the project here was narrowed down the following experiments to αGITR10-αPD-L1 and αGITR1-αPD-L1 tBsAbs. The cell based enzyme-linked immunosorbent assay (ELISA) was used to test the αGITR1-αPD-L1, αGITR10-αPD-L1 at different concentrations against GITR+ CF2 cells to analyze their reactivity. As shown in FIG. 20, reactivity to GITR+ CF2 could be observed for αGITR1-αPD-L1 and αGITR10-αPD-L1 antibodies. The OD value of αGITR1-αPD-L1 and αGITR10-αPD-L1 depend on their respective concentrations. Consistent with the expectations, the stronger signal was measured at the higher concentration; then gradually diminished as the concentration decreased.

The signal intensity of αGITR10-αPD-L1 is superior to that by αGITR1-αPD-L1 at all concentrations. Surprisingly, the negative control F10-αPD-L1 antibody not only shows absorbance but also seems to behave in a concentration-dependent manner. For αGITR1-αPD-L1 and F10-αPD-L1, no readout signal was detected below the threshold of 0.1235 mg/mL. Overall, the standard deviations of the mean were exceptionally high.

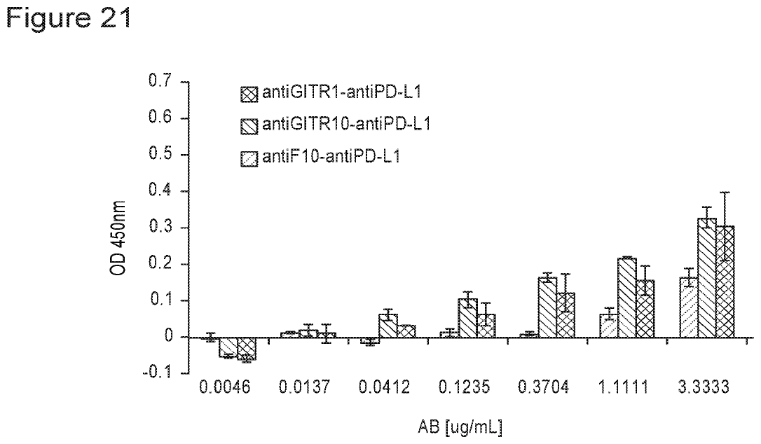

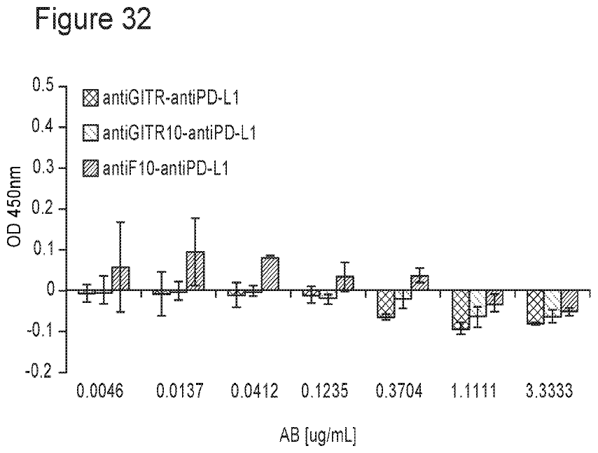

Due to the surprising results of the previous ELISA (see FIG. 20), the experiment was repeated. The set-up remained identical, with the exception that the GITR+ CF2 cells were fixed with 8% paraformaldehyde instead of acetone-methanol solution. Results of this second approach revealed similar signal readout observations of αGITR1-αPD-L1 and αGITR10-αPD-L1 antibodies, but with slightly higher absorption values (See, FIG. 21). However, the F10-αPD-L1 antibody continues to display signal activity and its absorbance is still dependent on the concentration used. The tBsAbs showed no binding when incubated with GITR-CF2 cells. See, FIG. 32.

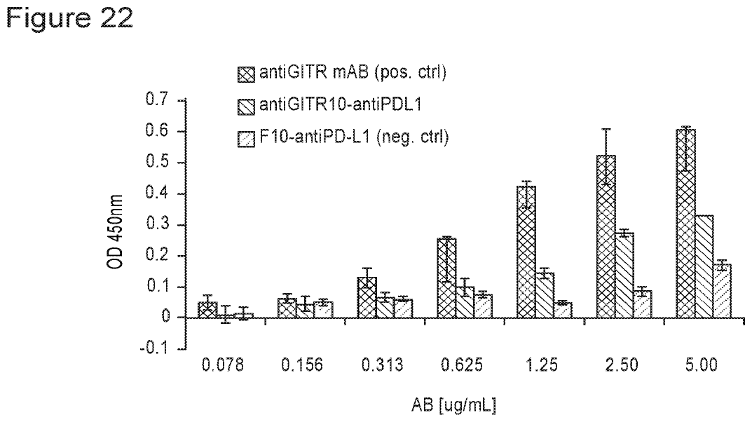

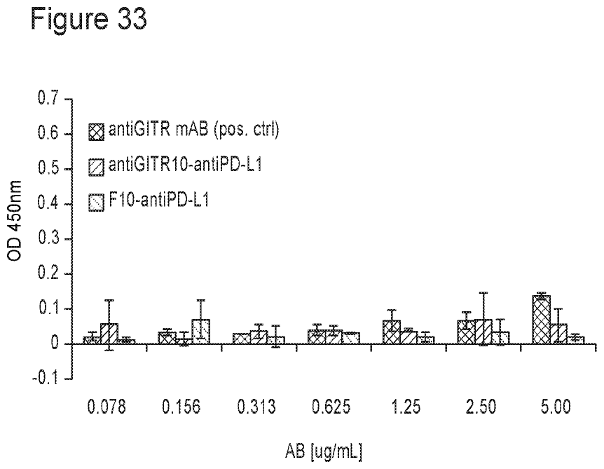

A third cell-based ELISA was performed to compare αGITR10-αPD-L1 antibody to a commercial αGITR IgG. Reactivity of both antibodies was observed in GITR+CF2 cells (FIG. 22) but not GITR-CF2 cells (See, FIG. 33). Again, the results of the αGITR10- αPD-L1 matched previous recorded data. The signal intensity of the αGITR mAb is superior to that by αGITR10-αPD-L1 at all concentrations. Surprisingly, at higher concentrations, no saturation of the signal readout could be observed. The control antibody F10-αPD-L1 (neg. control) showed concentration-dependent signal activity for GITR+CF2 cells but not for CF2 cells (without GITR+ expression). See, FIG. 33.

Flow Cytometry Analysis of αGITR-αPD-L1 BsAbs on GITR− Cells

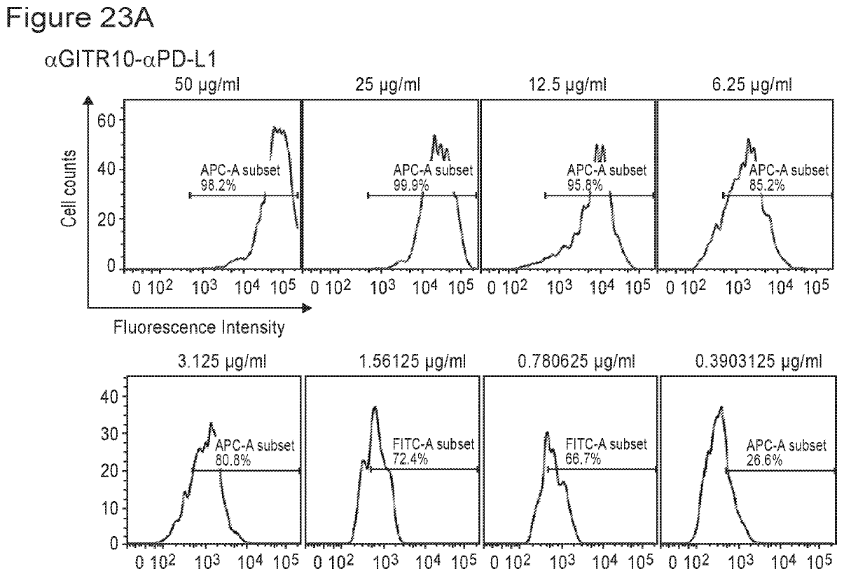

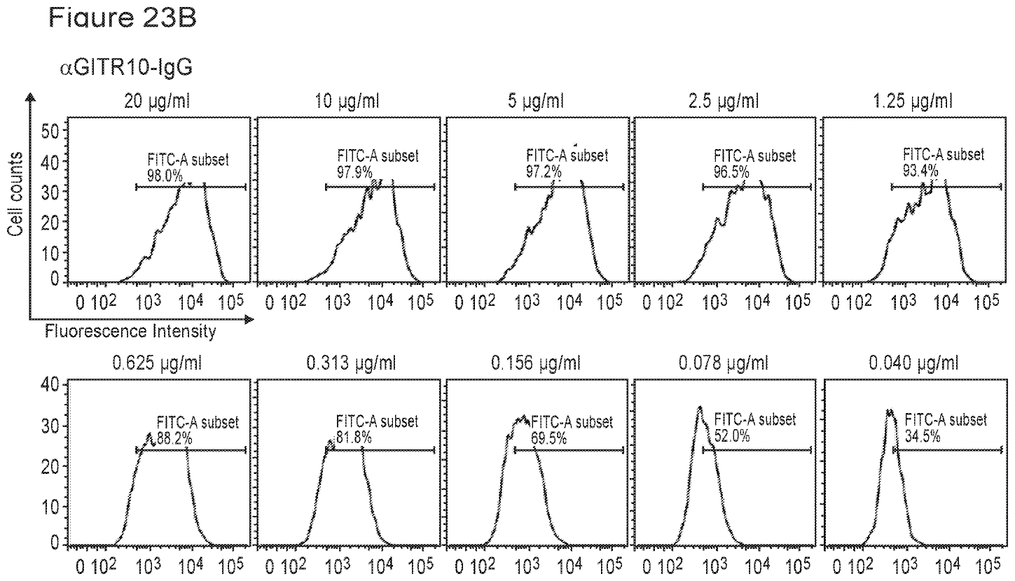

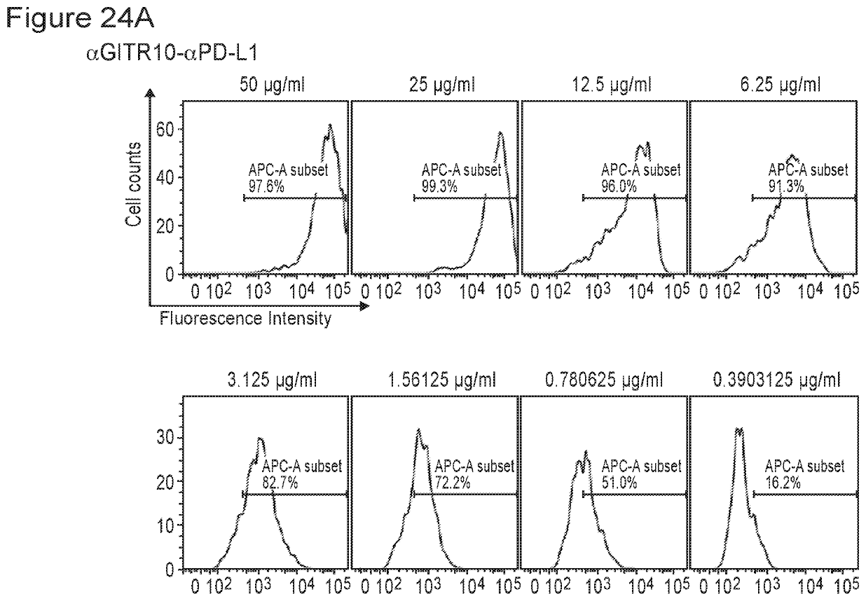

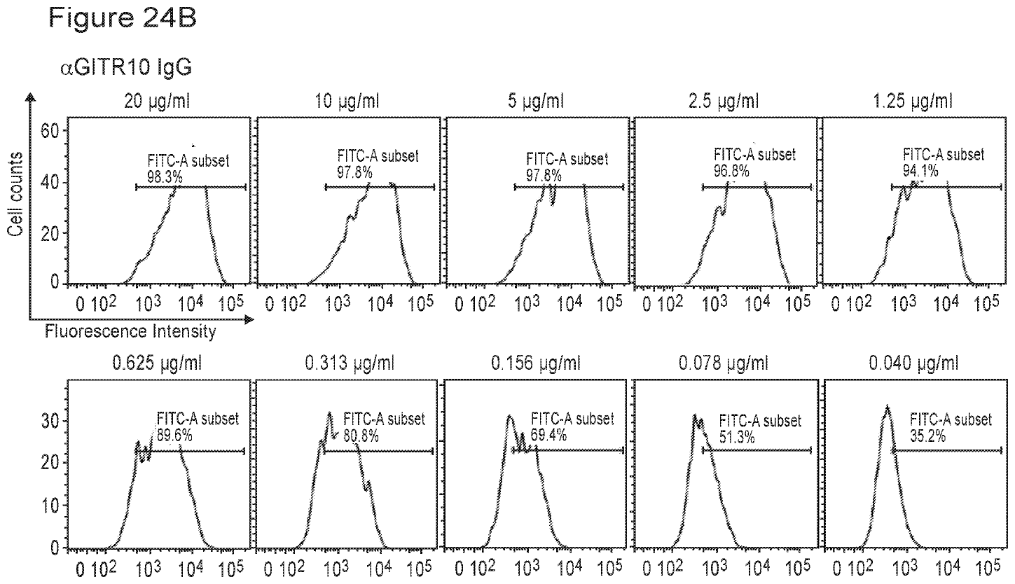

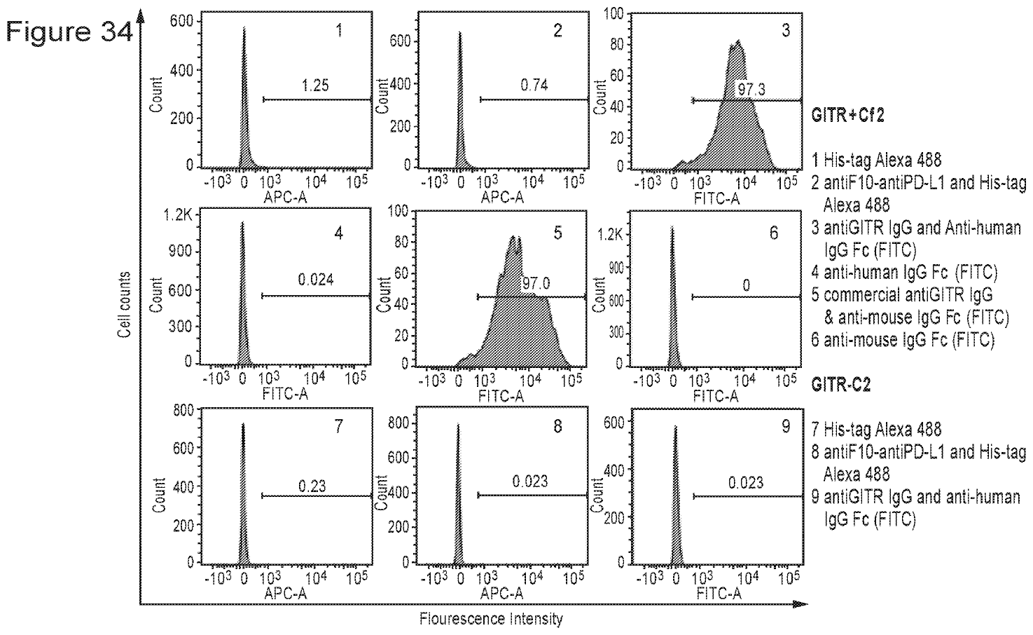

Flow cytometry analysis assesses the binding of αGITR1-αPD-L1 and αGITR10-αPD-L1 antibodies to GITR+CF2 cells (FIGS. 23 and 24). The results show, that both antibodies (co-stained with an APC-labeled His-Tag Alexa Fluor 488) can bind specifically the GITR+CF2. Further, the tBsAbs had no reactivity against GITR−CF2 (FIG. 34) Note that some non-specific binding is caused by the secondary antibodies as shown in the controls (FIG. 34). The comparison of the two antibodies to each other shows that they display similar binding under the identical conditions. Therefore, only the αGITR10-αPD-L1 tBsAb was selected for further characterization. The standard measurement of the αGITR1 IgG and αGITR10 IgG revealed similar binding properties when compared to the tBsAbs.

### Example 7: Isolation and Characterization of αGITR-αPD-L1 with CH2 bsAb

Generation of Bacterial Expression Vector

In previous studies, the αGITR10 mAb was proven to exhibit the best characteristics, which is why αGITR10-αPD-L1 was chosen as the expression vector for the engineering of the new construct containing a CH2 domain. The vector was generated according to the cloning strategy described above, resulting in the gene order V_{H}GITR-linker-V_{L}GITR-linker-hinge-CH2-linker-V_{H}PD-L1-linker-V_{L}PD-L1.

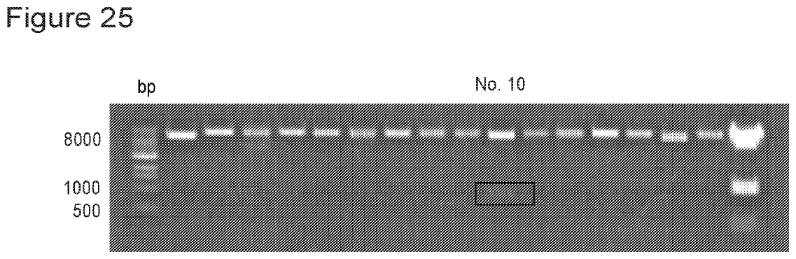

Site-directed mutagenesis enabled introduction of a HindIII restriction site into the recipient pcDNA 3.4 vector between the IgG1 Hinge region and the linker (GGGGS)₆ (SEQ ID NO: 1909). After transformation into E. coli strain XL10-Gold® Ultracompetent cells, 16 clones were picked then underwent DNA purification. A restriction enzyme digestion analysis using HindIII and BamHI restriction enzymes, displayed on a 1% agarose gel, tested for the correct introduction of HindIII restriction site (See FIG. 25). Of the 16 clones, only clone No. 10 showed two bands. The size of the smaller band clustered between 500 and 1000 bp, corresponds to the theoretical size of HindIII and BamHI digestion (800 bp). Since HindIII and BamHI represent unique restriction sites in the plasmid, this result indicated a successful introduction of HindIII into the DNA of cells in clone No. 10.

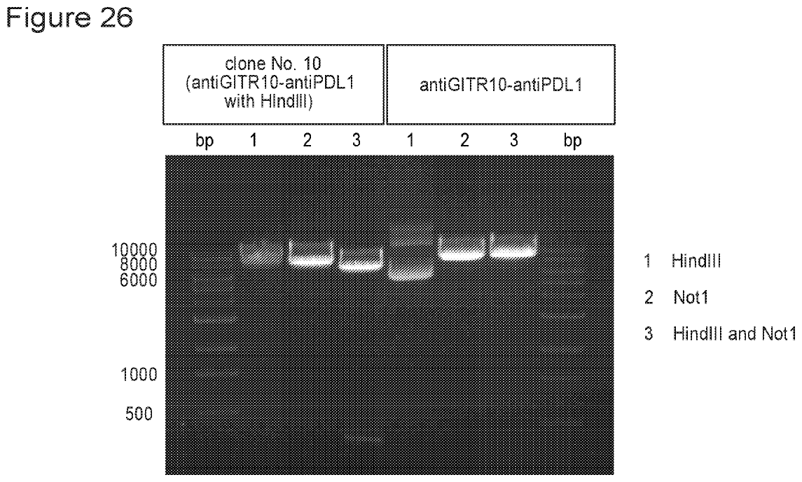

An additional gel analysis of clone No. 10 was undertaken to compare it to the original GITR10-PDL1 (that does not contain the HindIII restriction site); see FIG. 26. Clone No. 10 (GITR10-PDL1 with HindIII) and GITR10-PDL1 (without HindIII) each individually underwent three digestions. The first digestion was performed with only HindIII restriction enzyme, a second one with only NotI restriction enzyme and a third digestion with both HindIII and NotI restriction enzymes. The digestions of clone No.10 with a single enzyme resulted in open-circular conformation and were clustered around 8000 bp. In contrast, the double digestion of clone No. 10 with HindIII and NotI restriction enzymes resulted in two bands. The lower band is clustered below 500 bp and corresponds to the theoretical calculated value for the HindIII/NotI digestion fragment (117 bp). The αGITR10-αPD-L1 plasmid does not contain a HindIII restriction site, which is why the gel analysis of HindIII single digestion revealed, as anticipated, the supercoiled plasmid DNA. These results strongly suggest the correct introduction of the HindIII restriction site.

The sequencing results (Genewiz) of clone No. 10 confirmed the correct introduction of HindIII between the hinge and the linker domain. However, deletion of five linker-repeats of the total six (GGGGS (SEQ ID NO: 1484)) repeats occurred during site directed mutagenesis. The new construct consequently exhibited only one repeat of the linker sequence rather than six linker repeats. Nevertheless, it was decided to continue plasmid construction with this new created plasmid containing a hinge region followed by one single linker repeat (GGGGS (SEQ ID NO: 1484)).

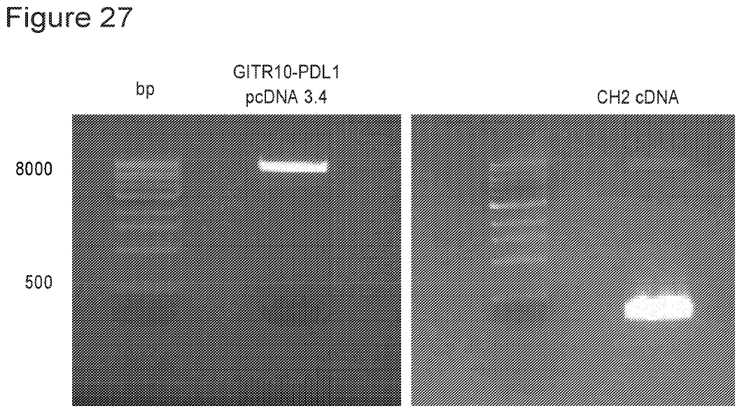

Two primers (forward and reverse) were designed to isolate the CH2 domain from an IgG1 plasmid. Each primer included a HindIII restriction site. The recipient vector GITR10-PDL1 (containing the HindIII site) and the CH2 fragment were single digested with the HindIII restriction enzyme and analyzed in a 1% agarose gel (FIG. 27). Both digestions resulted in a fragment size that matches the theoretical calculated values: 7.5 bp for the recipient vector GITR10-PDL1 with HindIII and 350 bp for the CH2 fragment.

Therefore, the pcDNA 3.4 expression vector αGITR10-αPD-L1 with CH2 was successfully constructed and confirmed by sequencing (Genewiz).

SDS-PAGE Analysis

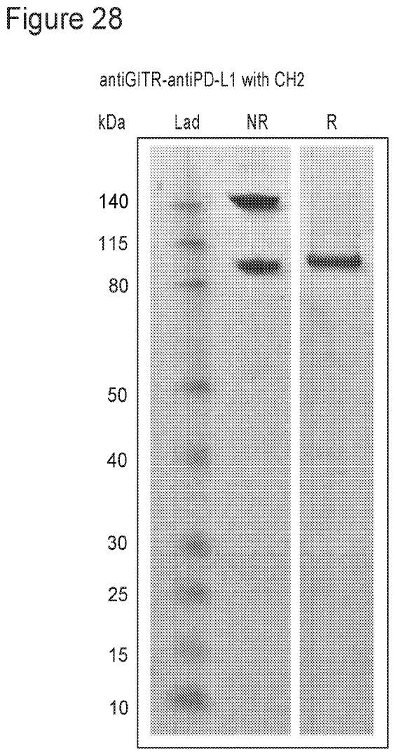

The αGITR10-αPD-L1 with CH2 protein was expressed in 293T HEK cells and isolated via Ni-NTA purification. A total of 100 mL culture media resulted in 200 ng protein yield (NanoDrop analysis). The purity of the GITR10-PDL1 with CH2 tBsAb was analyzed by SDS-PAGE (FIG. 28). In total 3 μg of protein sample was loaded on the gel, separated electrophoretically and stained with Coomassie blue. Notable, under non-reducing conditions there are two bands that particularly draw attention. The upper band lies slightly above 140 kDa. The quantitative predominance of this band and the proximity of its apparent molecular size to that of the αGITR10-αPD-L1 with CH2 tBsAb (150 kDa) propose the successful antibody production. The lower band has an apparent molecular weight of 80 kDa and thus, may account for a substantial amount of tandem scFv containing the CH2 (75 kDa) that did not dimerize. Under reducing conditions only one band can be seen at 80 kDa, which suggests that the disulfide bond reduction of the tBsAb into tandem scFv (75 kDa).

Cell-Based ELISA of αGITR-αPD-L1 with CH2 Tetrameric Bispecific Antibody (tBsAb) on GITR⁺ CF2

The cell-based enzyme-linked immunosorbent assay (ELISA) was performed to test the αGITR10-αPD-L1 with CH2 at different concentrations against GITR⁺ CF2 to analyze their signal intensity.

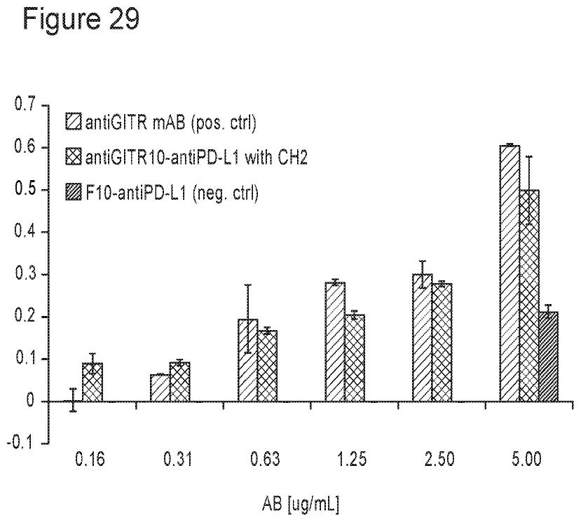

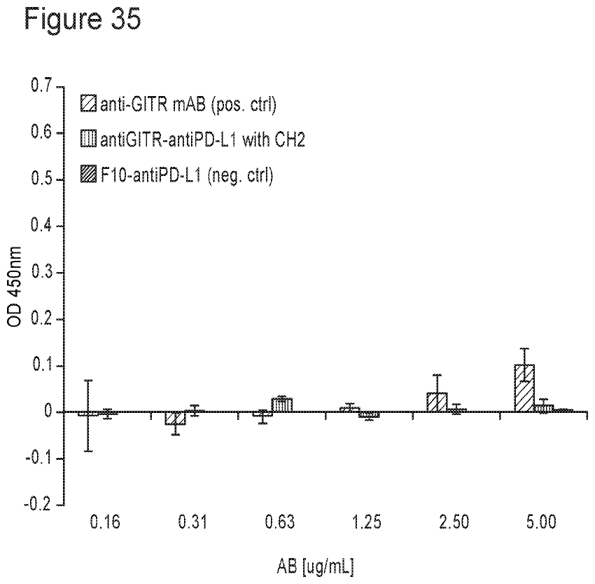

As shown in FIG. 29, reactivity to GITR⁺ CF2 could be observed in αGITR10-αPD-L1 with CH2 antibodies, while no unspecific stickiness to GITR⁻ CF2 was noted; see FIG. 35. The OD value of αGITR10-αPD-L1 with CH2 depends on their respective concentrations. Consistent with the expectations, the strongest signal was measured at the highest concentration; then gradually diminished as the concentration decreased. The signal intensity of the αGITR IgG is superior to that of αGITR10-αPD-L1 with CH2 at most concentrations. Surprisingly, at higher concentrations, no saturation of the signal readout could be observed. The control antibody (F10-αPD-L1) was tested at highest concentration (5 μg/mL) and had, as previously seen (FIGS. 22 and 21), some reactivity.

### Example 8: Functional Studies of αGITR-αPD-L1 with CH2 BsAb

In an initial attempt to establish functional data, complement-dependent cytotoxicity (CDC) and antibody dependent cellular cytotoxicity (ADCC) was tested for the αGITR-αPD-L1 with CH2 BsAbs. However, the results were inconclusive.

ADCC Reporter Assay of αGITR-αPD-L1 with CH2 BsAb on GITR+ CF2

The αGITR10-αPD-L1 with CH2 BsAb was tested for ADCC activity using GITR+CF2 cells (target cells) and the WIL2-S (effector cells) (E/T=5:1). Antibody biological activity in ADCC was quantified through the luciferase produced as a result of NFAT pathway activation and its activity in the effector cell was quantified with luminescence readout. In the ADCC analysis, the αGITR10-αPD-L1 and αGITR10-αPD-L1 with CH2 displayed surprising results (FIG. 30). The negative control F10-αPD-L1 displayed similar signal intensity for ADCC compared to solely target and effector cells and is unbiased to variable concentrations. The positive control αGITR IgG on the other hand, exhibited, as expected, increasing values with higher concentrations. Surprisingly, for the αGITR10-αPD-L1 and αGITR10-αPD-L1 with CH2 the ADCC signal intensity decreased with higher concentrations and was substantially lower than the signal of solely target and effector cells at 20 μg/mL tBsAbs.

The ADCC activity ofaaGITR10-αPD-L1 with CH2 and was measures at variable concentrations. All antibodies were serially diluted (1:2), starting the highest concentration at 20 mg/mL until 0.02 mg/mL and tested against 20,000 GITR+CF2 cells per well. The ratio of effector cells (GITR+CF2) to target cells (Wils-2) was 5:1. The αGITR IgG represents the positive control and F10-αPD-L1 the negative control. The vertical axis represents the raw value of luciferase activity in the effector cell quantified with luminescence readout. Each sample was run in triplicates at every concentration; the mean standard deviation is indicated in brackets. The background of GITR+CF2 cells in RPMI medium was subtracted from the obtained values.

CDC Analysis of αGITR10-αPD-L1 BsAb with CH2 on GITR+CF2 Cells

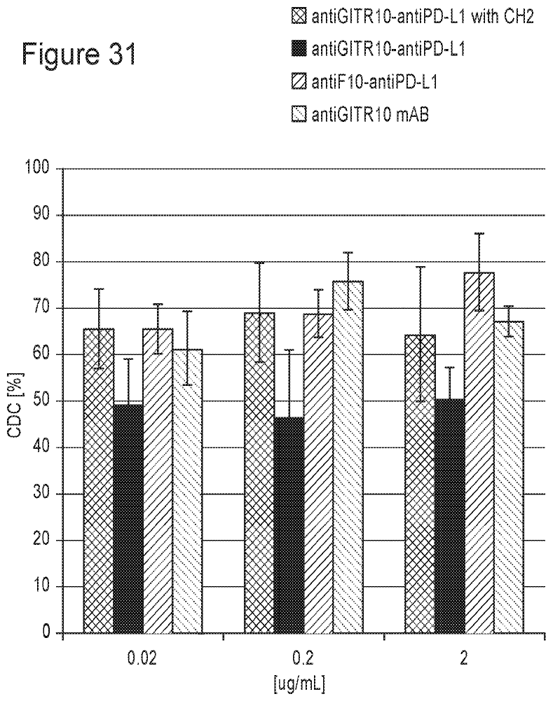

The αGITR10-αPD-L1 with CH2 antibody was tested for complement-dependent cytotoxicity against CF2 cells expressing the GITR by measuring the amount of fluorescent CellTox Green bound to comprised DNA. The percentage of lysis is calculated as the ratio of the intensity of the signal obtained from the sample, to the intensity of the signal from fully lysed GITR+ CF2 cells (FIG. 31).

The negative control F10-αPD-L1 BsAb displayed similar percentages of cytotoxicity as the positive control αGITR IgG. αGITR10-αPD-L1 with CH2 exhibited similar cytotoxicity levels at all concentrations ranging between 65% and 70% and did not appear to be concentration dependent. Neither of the measured antibodies had a substantial higher percentage of cellular cytotoxicity. These findings largely contradict the expected outcome; a possible reason for these incongruities is a potentially low viability of the used GITR+CF2 cells.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A tetravalent antibody molecule, wherein the tetravalent antibody is a dimer of a bispecific scFv fragment comprising a first binding site for a first antigen, a second binding site for a second antigen, wherein the two binding sites are joined together via a linker domain.

2. The tetravalent antibody molecule of claim 1, wherein the scFv fragment is a tandem scFv.

3. The tetravalent antibody molecule of claim 1, wherein the linker domain comprises an immunoglobulin hinge region amino acid sequence.

4. The tetravalent antibody molecule of claim 3, wherein the hinge region is an IgG1, IgG2, an IgG3, or an IgG4 hinge region.

5. The tetravalent antibody molecule of claim 3 or claim 4, wherein the immunoglobulin hinge region amino acid sequence is flanked by a flexible linker amino acid sequence.

6. The tetravalent antibody molecule of claim 5, wherein the flexible linker amino acid sequence comprises the amino acid sequence (GGGS)_{X1-6} (SEQ ID NO: 1906) (GGGGS)_{X1-6} (SEQ ID NO: 1907), or GSAGSAAGSGEF (SEQ ID NO: 1908).

7. The tetravalent antibody molecule of claim 1 or claim 2, wherein the linker domain comprises at least a portion of an immunoglobulin Fc domain.

8. The tetravalent antibody molecule of claim 7, wherein the Fc domain is an IgG1, an IgG2, an IgG3, or an IgG4 Fc domain.

9. The tetravalent antibody molecule of claim 7 or claim 8, wherein the at least a portion of an immunoglobulin Fc domain is a CH2 domain.

10. The tetravalent antibody molecule of any one of claims 7 to 9, wherein the Fc domain is linked to the C-terminus of an immunoglobulin hinge region amino acid sequence.

11. The tetravalent antibody molecule of claim 10, wherein the hinge region is an IgG1, an IgG2, an IgG3, or an IgG4 hinge region.

12. The tetravalent antibody molecule of claim 9 or claim 10, wherein the linker domain comprises a flexible linker amino acid sequence at one terminus or at both termini.

13. The tetravalent antibody molecule of claim 12, wherein each flexible linker amino acid sequence independently comprises the amino acid sequence (GGGS)_{X1-6} (SEQ ID NO: 1906) (GGGGS)_{X1-6} (SEQ ID NO: 1907) or GSAGSAAGSGEF (SEQ ID NO: 1908).

14. A nucleic acid construct comprising nucleic acid molecules encoding:
a light chain variable region and a heavy chain variable region of an antibody that can specifically bind to a first antigen;
a light chain variable region and a heavy chain variable region of an antibody that can specifically bind to a second antigen; and
a linker domain.

15. The nucleic acid construct of claim 14, wherein the linker domain comprises an immunoglobulin hinge region amino acid sequence.

16. The nucleic acid construct of claim 15, wherein the hinge region is an IgG1, an IgG2, an IgG3, or an IgG4 hinge region.

17. The nucleic acid construct of any one of claims 14 to 16, wherein the linker domain comprises at least a portion of an immunoglobulin Fc domain.

18. The nucleic acid construct of claim 17, wherein the Fc domain is an IgG1, an IgG2, an IgG3, or an IgG4 Fc domain.

19. The nucleic acid construct of claim 17 or claim 18, wherein the at least a portion of an immunoglobulin Fc domain is a CH2 domain.

20. The nucleic acid construct of any one of claims 17 to 19, wherein the Fc domain is linked to the C-terminus of the hinge region.

21. The nucleic acid construct of any one of claims 14 to 20, wherein the linker domain comprises a flexible linker amino acid sequence at one terminus or at both termini.

22. The nucleic acid construct of claim 21, wherein each flexible linker amino acid sequence independently comprises the amino acid sequence (GGGS)_{X1-6} (SEQ ID NO: 1906) (GGGGS)_{X1-6} (SEQ ID NO: 1907) or GSAGSAAGSGEF (SEQ ID NO: 1908).

23. A vector comprising the nucleic acid construct of any one of claims 14 to 22.

24. A host cell comprising the vector of claim 23.

25. The host cell of claim 24, wherein the cell is a T-cell, a B-cell, a follicular T-cell, or an NK-cell.

26. A chimeric antigen receptor (CAR) comprising an intracellular signaling domain, a transmembrane domain and an extracellular domain comprising the tetravalent antibody molecule of claim 1.

27. The CAR of claim 26, wherein the transmembrane domain further comprises a stalk region positioned between the extracellular domain and the transmembrane domain.

28. The CAR or claim 26, wherein the transmembrane domain comprises CD28.

29. The CAR of claim 26, further comprising one or more additional costimulatory molecules positioned between the transmembrane domain and the intracellular signaling domain.

30. The CAR of claim 29, wherein the costimulatory molecules is CD28, 4-1BB, ICOS, or OX40.

31. The CAR of claim 26, wherein the intracellular signaling domain comprises a CD3 zeta chain.

32. A genetically engineered cell which expresses and bears on the cell surface membrane the chimeric antigen receptor of any one of claims 26-31.

33. The genetically engineered cell of claim 32, wherein the cell is a T-cell or an NK cell.

34. The genetically engineered cell of claim 33, wherein the T cell is CD4+ or CD8+.

35. The genetically engineered cell of claim 34, which comprises a mixed population of CD4+ and CD8 cells+.

36. A method for treating a disease or disorder comprising administering the tetravalent antibody molecule of any one of claims 1 to 12.

37. The method of claim 36, wherein the disease or disorder is a CNS-related disease or disorder.

38. The method of claim 37, wherein the CNS-related disease or disorder is a CNS cancer.

39. The method of claim 38, wherein the CNS cancer is a Glioblastoma (GBM).

40. The method of claim 37, wherein the CNS-related disease or disorder is a neurodegenerative disease.

41. The method of claim 40, wherein the neurodegenerative disease is Amyotrophic Lateral Sclerosis, Parkinson's Disease, Alzheimer's Disease, or Huntington's Disease.

42. The method of any one of claims 37 to 42, wherein the tetravalent antibody molecule recognizes and/or is bound by a CNS transport receptor.

43. The method of claim 42, wherein the CNS transport receptor is a transferrin receptor (TfR), VCAM-1, CD98hc, or an insulin receptor.

44. The method of any one of claims 37 to 42, wherein the tetravalent antibody molecule augments transport across the blood brain barrier.
